(19) United States
(12) Patent Application Publication
(10) Pub. No.: US 2025/0134966 A1
(43) Pub. Date: May 1, 2025

(54) Reducing or Inhibiting Tissue Damage Using Hyaluronidase Administration

(71) Applicant: Med Progress, LLC, Newport Beach, CA (US)

(72) Inventor: Steve Yoelin, Newport Beach, CA (US)

(73) Assignee: Med Progress, LLC, Newport Beach, CA (US)

(21) Appl. No.: 18/693,468

(22) PCT Filed: Sep. 25, 2022
(86) PCT No.: PCT/US2022/076987

§ 371 Date: Mar. 19, 2024

Related U.S. Application Data
(60) Provisional application No. 63/248,447, filed on Sep. 25, 2021.

(51) Int. Cl. A61K 38/47; A61P 9/00

(52) U.S. Cl. CPC A61K 38/47; A61P 9/00; C12Y 302/01035

(57) ABSTRACT

The present disclosure discloses compositions comprising a hyper-concentrated hyaluronidase, devices comprising such compositions, as well as methods and uses employing such compositions and devices to reduce or eliminate a hyaluronic acid-induced blockage of one or more blood vessels supplying blood to an area of tissue of an individual; methods and uses for employing such compositions and devices to reduce or inhibit a vascular occlusion in an area of tissue of an individual; and methods and uses for employing such compositions and devices to reduce or inhibit a hyaluronic acid-induced loss of tissue of an individual.

This application is a 35 U.S.C. § 371 U.S. national stage patent application which claims the benefit of priority and is entitled to the filing date of International Patent Application PCT/US2022/076987, filed Sep. 25, 2022, an international patent application which claims the benefit of priority and is entitled to the filing date pursuant to 35 U.S.C. § 119 (e) of U.S. Provisional Patent Application 63/248,447, filed Sep. 25, 2021, the content of which is hereby incorporated by reference in its entirety.

### FIELD OF THE INVENTION

The present invention relates to the reduction or treatment of tissue damage caused by vascular occlusions in tissue due to the administration of a hyaluronic acid soft-tissue dermal filler.

### BACKGROUND

The use of fillers for augmentation of facial soft tissues in an individual has become one of the most commonly performed aesthetic procedures. For example, soft-tissue filler injection is the second most commonly performed cosmetic procedure following botulinum toxin treatment. The most commercially popular soft-tissue dermal fillers are ones employing hyaluronic acid.

Unfortunately, as the field of soft-tissue augmentation has become increasingly popular, reports of adverse events have increased. One of the more severe side effects is the blockage of blood flow at the sites of administration. Called vascular occlusion, the prevention of blood to tissue can lead to irreversible damage due to the lack of sufficient oxygenation and nutrient supply of the surrounding tissue, including, e.g., cellular necrosis, scarring, local neuronal desensitization or paralysis, and potential systemic complications as well. For example, a vascular occlusion of the dermal and epidermal layers of the skin following a soft-tissue augmentation procedure of the face and neck areas can lead to permanent disfiguration and functional loss of the affected tissue areas.

Unfortunately, there is no proven, consistently effective treatment for vascular occlusion following a soft-tissue filler injection. Therefore, when an occlusive event occurs within blood vessels of tissues, the damage inflicted can potentially be significant and permanent. Furthermore, even with early recognition of a vascular occlusion event, tissue damage associated with soft-tissue filler injections can be irreversible unless blood flow can be restored. Given the vulnerability of skin tissues to a filler-induced vascular occlusion event, methods for reducing or eliminating this blockage are needed.

### SUMMARY

The present disclosure provides such a solution for soft-tissue dermal fillers comprising hyaluronic acid. The compositions, devices, methods and uses disclosed herein administer a hyaluronidase to a region of tissue. Such administration can deliver a hyaluronidase into the blood vessels supplying the area of tissue in about 5 minutes to 30 minutes. This fast delivery of hyaluronidase enables this enzyme to reduce or eliminate the hyaluronic acid causing the vascular occlusion, thereby reducing or eliminating the risk of permanent damage to the tissue affected by the occlusion.

Aspects of the present specification disclose methods of reducing or eliminating a hyaluronic acid-induced blockage in one or more blood vessels supplying blood to an area of tissue in an individual in need thereof. The disclosed methods comprise administering a composition comprising a hyper-concentrated hyaluronidase to an individual in a manner that reduces or eliminates a hyaluronic acid-induced blockage in one or more blood vessels supplying blood to the area of tissue being affected by the blockage. In aspects, the disclosed methods administer composition comprising a hyper-concentrated hyaluronidase to a dermal portion of a skin region. In other aspects, the disclosed methods administer a composition comprising a hyper-concentrated hyaluronidase using a delivery system, such as, e.g., a manual or auto-injecting device. The disclosed methods can administer a composition comprising a hyper-concentrated hyaluronidase in a single- or multi-dose amount ranging from about 250 IU/mL to about 500,000 IU/ml of a hyaluronidase.

Aspects of the present specification disclose methods of reducing or inhibiting a vascular occlusion in an area of tissue of an individual in need thereof. The disclosed methods comprise administering a composition comprising a hyper-concentrated hyaluronidase to an individual in a manner that reduces or eliminates a hyaluronic acid-induced blockage in one or more blood vessels supplying blood to the area of tissue being affected by the blockage, thereby reducing or inhibiting the vascular occlusion. In aspects, the disclosed methods administer composition comprising a hyper-concentrated hyaluronidase to a dermal portion of a skin region. In other aspects, the disclosed methods administer a composition comprising a hyper-concentrated hyaluronidase using a delivery system, such as, e.g., a manual or auto-injecting device. The disclosed methods can administer a composition comprising a hyper-concentrated hyaluronidase in a single- or multi-dose amount ranging from about 250 IU/mL to about 500,000 IU/mL of a hyaluronidase.

Aspects of the present specification disclose methods of reducing or inhibiting hyaluronic acid-induced loss of tissue in an individual in need thereof. The disclosed methods comprise administering a composition comprising a hyper-concentrated hyaluronidase to an individual in a manner that reduces or eliminates a hyaluronic acid-induced blockage in one or more blood vessels supplying blood to an area of tissue being affected by the blockage, thereby reducing or inhibiting the hyaluronic acid-induced loss of tissue in the individual. In aspects, the disclosed methods administer a composition comprising a hyper-concentrated hyaluronidase to a dermal portion of a skin region. In other aspects, the disclosed methods administer a composition comprising a hyper-concentrated hyaluronidase using a delivery system, such as, e.g., a manual or auto-injecting device. The disclosed methods can administer a composition comprising a hyper-concentrated hyaluronidase in a single- or multi-dose amount ranging from about 250 IU/mL to about 500,000 IU/mL of a hyaluronidase.

Other aspects of the present specification disclose a delivery system, such as, e.g., a manual or auto-injecting device, comprising a composition comprising a hyper-concentrated hyaluronidase. In aspects, the disclosed delivery system delivers a composition comprising a hyper-concentrated hyaluronidase to an area of tissue. The disclosed delivery system can administer a composition comprising a hyper-concentrated hyaluronidase in a single- or multi-dose amount ranging from about 250 IU/mL to about 500,000 IU/mL of a hyaluronidase.

Other aspects of the present specification disclose uses of a composition comprising a hyper-concentrated hyaluronidase. In aspects, the disclosed uses comprise a composition comprising a hyper-concentrated hyaluronidase contained in a delivery system, such as, e.g., a manual or auto-injecting device. In other aspects, the disclosed uses comprise a composition comprising a hyper-concentrated hyaluronidase contained in an auto-injecting device. In other aspects, the disclosed uses comprise a composition comprising a hyper-concentrated hyaluronidase contained in a manual injecting device. The disclosed uses can administer a composition comprising a hyper-concentrated hyaluronidase in a single- or multi-dose amount ranging from about 250 IU/mL to about 500,000 IU/mL of a hyaluronidase.

Other aspects of the present specification disclose uses of a delivery system, such as, e.g., a manual or auto-injecting device, comprising a composition comprising a hyper-concentrated hyaluronidase. In aspects, the disclosed uses comprise an auto-injecting device comprising a composition comprising a hyper-concentrated hyaluronidase. In aspects, the disclosed uses comprise a manual injecting device comprising a composition comprising a hyper-concentrated hyaluronidase. The disclosed uses can administer a composition comprising a hyper-concentrated hyaluronidase in a single- or multi-dose amount ranging from about 250 IU/mL to about 500,000 IU/mL of a hyaluronidase.

Other aspects of the present specification disclose uses of a composition comprising a hyper-concentrated hyaluronidase in the manufacture of a medicament. In aspects, the disclosed medicament comprises a composition comprising a hyper-concentrated hyaluronidase contained in a delivery system, such as, e.g., a manual or auto-injecting device. In other aspects, the disclosed medicament comprises a composition comprising a hyper-concentrated hyaluronidase contained in an auto-injecting device. In other aspects, the disclosed medicament comprises a composition comprising a hyper-concentrated hyaluronidase contained in a manual injecting device. The disclosed uses can administer a composition comprising a hyper-concentrated hyaluronidase in a single- or multi-dose amount ranging from about 250 IU/mL to about 500,000 IU/mL of a hyaluronidase.

Other aspects of the present specification disclose a composition comprising a hyper-concentrated hyaluronidase for use in inhibiting hyaluronic acid-induced blockage of one or more blood vessels supplying an area of tissue. In aspects, a composition comprising a hyper-concentrated hyaluronidase is contained in a delivery system, such as, e.g., a manual or auto-injecting device. In other aspects, a composition comprising a hyper-concentrated hyaluronidase is contained in an auto-injecting device. In other aspects, a composition comprising a hyper-concentrated hyaluronidase is contained in a manual injecting device. The disclosed compositions comprising a hyper-concentrated hyaluronidase can contain a single- or multi-dose amount ranging from about 250 IU/mL to about 500,000 IU/mL of a hyaluronidase.

Other aspects of the present specification disclose a delivery system, such as, e.g., a manual or auto-injecting device, comprising a composition comprising a hyper-concentrated hyaluronidase for use in inhibiting hyaluronic acid-induced blockage of one or more blood vessels supplying an area of tissue. The disclosed delivery system comprising a composition comprising a hyper-concentrated hyaluronidase can contain a single- or multi-dose amount ranging from about 250 IU/mL to about 500,000 IU/mL of a hyaluronidase.

### DETAILED DESCRIPTION

The skin is the outer covering of a mammalian body and guards the underlying muscles, bones, ligaments and internal organs. Typically the largest organ of the integumentary system, skin is composed of three primary layers: the epidermis, the dermis and the hypodermis. The epidermis is the outermost epithelial layer of the skin and forms the waterproof, protective wrap over the body's surface which also serves as a barrier to infection. The dermis is an epithelial layer of skin beneath the epidermis and serves to cushion the body from stress and strain. The hypodermis lies below the dermis and comprises loose connective tissue, adipose tissue and elastin. Although not composed of epithelial tissue, and thus not part of the skin, the hypodermis attaches the skin to underlying bone and muscle as well as supplies the skin with blood vessels and nerves.

Unfortunately, administration of a soft-tissue dermal filler, like a hyaluronic-acid filler, to the face can result in filler entering the skin circulation, which leads to blockage of one or more vessels supplying an area of tissue, such as the hypodermis, the dermis or the epidermis. Such blockage impedes or prevents blood flow, resulting in the deprivation of oxygen and nutrients to the area that was being supplied by the occluded vessel. If blood flow through the occluded vessel is not restored quickly, cellular ischemia and/or tissue damage with corresponding necrosis, local nerve damage, and systemic complications may result. These adverse events can happen rapidly, with permanent injury occurring if such vascular occlusions are left untreated or not treated in a timely manner.

Additionally, administration of a soft-tissue dermal filler, like a hyaluronic-acid filler, can cause hard-to-treat delayed hypersensitivity reactions. Such hypersensitivity can appear within a few weeks and can appear even years after administration. When a hypersensitivity reaction manifests, symptoms such as, e.g., pain, tenderness, unsightliness, and scars, can last for months, years, or even become permanent. The cause of these reactions is unclear, and current treatment options include oral antibiotics, oral steroids, steroid injections, low-dose hyaluronidase injections, and 5-fluorouracil (5-FU) injections, all of which may require numerous treatment cycles in combination with one another. However, such treatment combinations sometimes yield adverse events that do not occur when such treatments are administered individually.

Among the treatments above, the injection of hyaluronidase is widely viewed as one of the most important treatment options since hyaluronidase actually removes HA dermal fillers, which cause the reactions in question. Notably, only low-concentration hyaluronidase can currently be administered in humans in the United States. Unfortunately, low-concentration hyaluronidase may have limited efficacy in dissolving HA dermal fillers in these scenarios due to the inflammation induced by these HA dermal fillers, thereby likely necessitating the many treatment cycles of low-concentration hyaluronidase that are noted above. Hyper-concentration hyaluronidase, in contrast, would treat these types of hypersensitivity reactions quickly and efficiently. Patients and practitioners alike would welcome the introduction of a hyper-concentration hyaluronidase for the treatment of these hypersensitivity reactions in order to alleviate such hypersensitivity reactions and corresponding adverse events, including permanent adverse events, more effectively. This is especially true given the current efforts by manufacturers to create longer-lasting HA dermal fillers that are more resistant to hyaluronidase and, therefore, last longer in vivo. As a result, these longer-lasting products are more resistant to exogenous hyaluronidase treatments. Thus, it is important to make available hyper-concentration hyaluronidase in order to reverse unwanted effects from these longer-lasting products when needed.

Hyaluronidases are a family of enzymes that catalyze the degradation of hyaluronic acid. Thus, one possible solution to a hyaluronic acid-induced blockage of one or more vessels supplying blood to an area of tissue is to administer a hyaluronidase to the site of such blockage. However, there is currently no recognized way to deliver an effective amount of hyaluronidase to the circulation in a manner that would reduce or eliminate hyaluronic acid-induced blockage in one or more blood vessels supplying blood to an area of tissue being affected by the blockage.

One problem facing a hyaluronidase treatment of a hyaluronic acid-induced blockage of one or more vessels is the fact that hyaluronidase is highly liable and has a very short half-life. For example, studies have shown that the half-life of intravenous administration of hyaluronidase is about 2 minutes while the half-life of intradermal or subcutaneous administration of this enzyme is less than about 30 minutes. As such, hyaluronidase is rapidly lost when administered directly into the blood or into tissue. Additionally, standard concentrations of commercially available hyaluronidase range from 150 IU/mL to 200 IU/mL. This low concentration necessitates administration of a large volume of hyaluronidase, such as 40 mL or more, in order to reach a therapeutically effective amount to degrade a hyaluronic acid-induced blockage of one or more vessels. Administration of such volumes in the blood is simply impractical. For example, stocking multiple vials of hyaluronidase is extremely expensive for practitioners, especially due to the relatively short shelf life of the types of hyaluronidases that are currently commercially approved for human use. Even if sufficient quantities are available, the standard 150 IU/mL to 200 IU/mL concentration is too low to achieve a therapeutic effect since this low enzyme concentration rapidly degrades once it enters the blood. In addition, injection directly into blood vessels at the site of a hyaluronic acid-induced blockage is incredibly challenging even when an imaging device, such as, e.g., an ultrasound device is employed to facilitate visualization of these vessels as the affected vessels are very small in diameter; and, unfortunately, the vast majority of practitioners do not have any imaging devices in their offices. As a remedy to this lack of on-hand imaging devices, hyper-concentrated hyaluronidase will enable practitioners to reverse the types of blockages noted above while injecting fewer vials of hyaluronidase, and hyper-concentrated hyaluronidase will also help enable practitioners to be less precise with their placement of the hyaluronidase when treating HA-induced vascular occlusions.

Another problem facing a hyaluronidase treatment of a hyaluronic acid-induced blockage of one or more vessels pertains to establishing a concentration gradient large enough to induce enough hyaluronidase into the blood vessels so that an effective amount of the enzyme reaches the occluded area and breaks apart the hyaluronic acid causing the blockage. Standard concentrations of commercially available hyaluronidase are in the range of 150 IU/mL to 200 IU/mL, and these low concentrations of hyaluronidase, even when administered in high volumes, are insufficient to consistently establish a concentration gradient large enough to deliver a therapeutically effective amount of enzyme to degrade a hyaluronic acid-induced blockage of one or more vessels. During such administrations, only a fraction of the hyaluronidase administered is able to enter the blood system, and this low amount of hyaluronidase is quickly degraded. Moreover, the limited quantity of hyaluronidase that enters blood vessels containing the types of blockages noted above will have a very limited ability to completely dissolve such blockages due to such limited quantity.

Thus, what is needed is a composition comprising a hyper-concentrated hyaluronidase that can establish a concentration gradient large enough to facilitate sufficient amounts of hyaluronidase to enter the blood, reach the occluded area(s), and exert a therapeutic effect by degrading hyaluronic acid-induced blockage of one or more vessels. The disclosed compositions, devices, methods and uses achieve this desired effect.

The compositions, devices, methods and uses disclosed herein deliver an effective amount of hyaluronidase to one or more blood vessels in a manner that would reduce or eliminate hyaluronic acid-induced blockage in one or more blood vessels supplying blood to an area of tissue being affected by the blockage. Such reduction or elimination of hyaluronic acid-induced vascular occlusions involves the administration of a hyper-concentrated hyaluronidase to the area of tissue being affected by a hyaluronic acid-induced blockage of one or more blood vessels. A hyper-concentrated hyaluronidase overcomes the fast clearance rate of this enzyme in the blood, ensuring that an effective amount can reach the desired target area. Additionally, the hyper-concentration of this enzyme ensures that a sufficiently steep gradient is established that facilitates transport of sufficient amounts of hyaluronidase into the blood. Additionally, practitioners wouldn't need to inject as many rounds of hyaluronidase due to its higher concentration relative to the types of hyaluronidases that are currently commercially available in the U.S. This reduced number of injecting rounds with the highly concentrated hyaluronidase also enables patients suffering from HA occlusions to recover more quickly. This delivery of hyper-concentrated hyaluronidase enables this enzyme to reduce or eliminate the hyaluronic acid causing the vascular occlusion, thereby reducing or eliminating the risk of permanent damage to the tissue. In summary, patients suffering from HA occlusions would have a much greater chance of avoiding irreversible tissue damage resulting from HA occlusions in addition to a faster recovery if such patients are injected with hyper-concentrated hyaluronidase, rather than low-concentration hyaluronidase.

Aspects of the present specification disclose a pharmaceutical composition. A pharmaceutical composition disclosed herein refers to a therapeutically effective concentration of an active ingredient, such as, e.g., a hyper-concentrated hyaluronidase disclosed herein. Preferably, the pharmaceutical composition disclosed herein does not produce an adverse, allergic, or other untoward or unwanted reaction when administered to an individual. A pharmaceutical composition disclosed herein is useful for medical and veterinary applications. A pharmaceutical composition disclosed herein may be formulated as a liquid pharmaceutical composition or as a dried pharmaceutical composition, such as, e.g., a lyophilized or freeze-dried formulation.

A pharmaceutical composition disclosed herein may be administered alone to an individual or in combination with supplementary active compounds, agents, drugs and/or hormones. In some embodiments, a pharmaceutical composition disclosed herein further includes a local anesthetic. Non-limiting examples of a local anesthetic include, e.g., 3-(p-fluorobenzoyloxy) tropane, amylocaine, amoproxan (3,4,5-trimethoxybenzoyl), articaine, benzocaine, bupivacaine, butacaine, butamben, butanilicaine, carticaine, chloroprocaine, cinchocaine, (dibucaine), cocaine, cyclomethycaine, dimethocaine, etidocaine, α-eucaine, β-eucaine, hexylcaineisobucaine, levobupivacaine, lidocaine, lucaine, mepivacaine, meprylcaine, metabutethamine, metabutoxycaine, nitracaine, orthocaine, piperocaine, prilocaine, propoxycaine, procaine (novocaine), proxymetacaine, risocaine, ropivacaine, tetracaine, trimecaine, or any combination thereof.

Aspects of the present specification disclose a hyaluronidase. Hyaluronidase hydrolyses hyaluronic acid. According to their enzymatic mechanism, hyaluronidases are hyaluronoglucosidases (EC 3.2.1.35), i.e., they cleave the (1->4)-linkages between N-acetylglucosamine and glucuronate. The term “hyaluronidase” may also refer to hyaluronoglucuronidases (EC 3.2.1.36), which cleave (1->3)-linkages. Pharmacokinetics of hyaluronidase have been assessed in animal studies after intravitreal injection. Hyaluronidase can be commercially obtained from animals, where it is typically extracted from ovine or bovine testicles (Vitrase, Bausch Health Companies, Inc., Laval, Quebec, Canada), leeches, or bacteria. Hyaluronidase can also be obtained recombinantly, e.g., by genetically manipulating human recombinant DNA in Chinese hamster ovary cells (Hylenex, Halozyme Therapeutics, Inc., San Diego, CA). Recombinant hyaluronidase is easier to produce in large commercial quantities and easier to concentrate compared to animal-derived hyaluronidase formulations. In addition, human-derived recombinant hyaluronidase has a lower theoretical incidence of allergic issues. A commercially available hyper-concentrated hyaluronidase is manufactured by Creative BioMart, which manufactures this enzyme at concentrations ranging from 1,000 IU/mL to 50,000 IU/mL where one unit is defined as causing a rate of change of OD 0.33/minute in the amount of hyaluronic acid at pH 5.7 at 37° C. when measured in a spectrophotometer at 600 nm.

The present specification discloses, in part, a therapeutically effective amount. A therapeutically effective amount of a hyaluronidase is an amount sufficient to reduce or eliminate a hyaluronic acid-induced blockage in one or more blood vessels of a tissue. In aspects of this embodiment, a therapeutically effective amount of a hyaluronidase is an amount sufficient to reduce one or more physiological conditions or symptoms associated with a hyaluronic acid-induced blockage in one or more blood vessels of a tissue or an amount sufficient to protect the individual against one or more physiological conditions or symptoms associated with a hyaluronic acid-induced blockage in one or more blood vessels of a tissue. As used herein, the term “therapeutically effective amount” includes the terms “amount sufficient”, “therapeutically sufficient amount”, “effective amount”, “effective dose”, and “therapeutically effective dose” and refers to the minimum amount of a hyaluronidase necessary to achieve the desired therapeutic effect and includes an amount sufficient to reduce or inhibit one or more physiological conditions or symptoms associated with a hyaluronic acid-induced blockage in one or more blood vessels of a tissue.

In aspects of this embodiment, a therapeutically effective amount of a hyper-concentrated hyaluronidase disclosed herein reduces or inhibits one or more physiological conditions or symptoms associated with a hyaluronic acid-induced blockage in one or more blood vessels of a tissue by, e.g., at least 10%, at least 20%, at least 30%, at least 40%, at least 50%, at least 60%, at least 70%, at least 80%, at least 90% or at least 100%. In other aspects of this embodiment, an effective amount of a hyper-concentrated hyaluronidase disclosed herein reduces or inhibits one or more physiological conditions or symptoms associated with a hyaluronic acid-induced blockage in one or more blood vessels of a tissue by, e.g., at most 10%, at most 20%, at most 30%, at most 40%, at most 50%, at most 60%, at most 70%, at most 80%, at most 90% or at most 100%. In yet other aspects of this embodiment, an effective amount of a hyper-concentrated hyaluronidase disclosed herein reduces or inhibits one or more physiological conditions or symptoms associated with a hyaluronic acid-induced blockage in one or more blood vessels of a tissue by, e.g., about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 20% to about 100%, about 20% to about 90%, about 20% to about 80%, about 20% to about 70%, about 20% to about 60%, about 20% to about 50%, about 20% to about 40%, about 20% to about 30%, about 30% to about 100%, about 30% to about 90%, about 30% to about 80%, about 30% to about 70%, about 30% to about 60%, about 30% to about 50%, about 30% to about 40%, about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%. In still other aspects of this embodiment, an effective amount of a hyper-concentrated hyaluronidase disclosed herein reduces or inhibits one or more physiological conditions or symptoms associated with a hyaluronic acid-induced blockage in one or more blood vessels of a tissue for, e.g., at least one week, at least one month, at least two months, at least three months, at least four months, at least five months, at least six months, at least seven months, at least eight months, at least nine months, at least ten months, at least eleven months, or at least twelve months.

In aspects of this embodiment, a therapeutically effective amount of a hyper-concentrated hyaluronidase disclosed herein restores or maintains one or more qualitative or quantitative aspects of normal blood circulation by, e.g., at least 10%, at least 20%, at least 30%, at least 40%, at least 50%, at least 60%, at least 70%, at least 80%, at least 90% or at least 100%. In other aspects of this embodiment, an effective amount of a hyper-concentrated hyaluronidase disclosed herein restores or maintains one or more qualitative or quantitative aspects of normal blood circulation by, e.g., at most 10%, at most 20%, at most 30%, at most 40%, at most 50%, at most 60%, at most 70%, at most 80%, at most 90% or at most 100%. In yet other aspects of this embodiment, an effective amount of a hyper-concentrated hyaluronidase disclosed herein restores or maintains one or more qualitative or quantitative aspects of normal blood circulation by, e.g., about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 20% to about 100%, about 20% to about 90%, about 20% to about 80%, about 20% to about 70%, about 20% to about 60%, about 20% to about 50%, about 20% to about 40%, about 20% to about 30%, about 30% to about 100%, about 30% to about 90%, about 30% to about 80%, about 30% to about 70%, about 30% to about 60%, about 30% to about 50%, about 30% to about 40%, about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%. In still other aspects of this embodiment, an effective amount of a hyper-concentrated hyaluronidase disclosed herein restores or maintains one or more qualitative or quantitative aspects of normal blood circulation for, e.g., at least one week, at least one month, at least two months, at least three months, at least four months, at least five months, at least six months, at least seven months, at least eight months, at least nine months, at least ten months, at least eleven months, or at least twelve months.

The actual therapeutic effective amount of a hyper-concentrated hyaluronidase disclosed herein to be used or administered to an individual can be determined by a person of ordinary skill in the art by taking into account factors that include, without limitation, the type of hyaluronic acid-induced blockage, the particular physiological conditions or symptoms associated with a hyaluronic acid-induced blockage, the cause of a hyaluronic acid-induced blockage, the severity of a hyaluronic acid-induced blockage, the degree of relief desired for a hyaluronic acid-induced blockage, the duration of relief desired for a hyaluronic acid-induced blockage, the particular soft-tissue dermal filler used, the rate of excretion of the particular hyaluronidase used, the pharmacodynamics of the particular hyaluronidase used, the nature of the other compounds to be included in the therapy, the particular route of administration used, the particular characteristics, history and risk factors of the individual, such as, e.g., age, weight, general health and the like, or any combination thereof. It is known by a person of ordinary skill in the art that an effective amount of a hyper-concentrated hyaluronidase disclosed herein can be extrapolated from in-vitro assays and in-vivo administration studies using animal models prior to administration to humans. Variations in dosage levels can be adjusted using standard empirical routines of optimization, which are well-known to a person of ordinary skill in the art. The precise therapeutically effective dosage levels and patterns are preferably determined by the attending healthcare professional in consideration of the above-identified factors.

Depending on the concentration of hyper-concentrated hyaluronidase in a composition disclosed herein, dosing can be a single-dose administration or multiple-dose administration. Typically, when formulated as a single dose, the concentration of hyper-concentrated hyaluronidase in a composition disclosed herein is one that effectively reduces or eliminates a hyaluronic acid-induced blockage in one or more blood vessels of an area of tissue being affected by the blockage. When a composition disclosed herein is formulated for multi-dose administration, the total amount of hyper-concentrated hyaluronidase cumulatively administered by the multiple doses is one that effectively reduces or eliminates a hyaluronic acid-induced blockage in one or more blood vessels of an area of tissue being affected by the blockage.

A composition disclosed herein can comprise a hyper-concentrated hyaluronidase in a concentration between about 250 IU/mL to about 500,000 IU/mL. In aspects of this embodiment, a composition disclosed herein comprises a hyaluronidase in a concentration of, e.g., at least 25,000 IU/mL, at least 50,000 IU/mL, at least 75,000 IU/mL, at least 100,000 IU/mL, at least 125,000 IU/mL, at least 150,000 IU/mL, at least 175,000 IU/mL, at least 200,000 IU/mL, at least 225,000 IU/mL, at least 250,000 IU/mL, at least 300,000 IU/mL, at least 350,000 IU/mL, at least 400,000 IU/mL, at least 450,000 IU/mL, or at least 500,000 IU/mL. In other aspects of this embodiment, a composition disclosed herein comprises a hyper-concentrated hyaluronidase in a concentration of, e.g., at most 25,000 IU/mL, at most 50,000 IU/mL, at most 75,000 IU/mL, at most 100,000 IU/mL, at most 125,000 IU/mL, at most 150,000 IU/mL, at most 175,000 IU/mL, at most 200,000 IU/mL, at most 225,000 IU/mL, at most 250,000 IU/mL, at most 300,000 IU/mL, at most 350,000 IU/mL, at most 400,000 IU/mL, at most 450,000 IU/mL, or at most 500,000 IU/mL.

In yet other aspects of this embodiment, a composition disclosed herein comprises a hyper-concentrated hyaluronidase in a concentration of, e.g., about 25,000 IU/mL to about 50,000 IU/mL, about 25,000 IU/mL to about 75,000 IU/mL, about 25,000 IU/mL to about 100,000 IU/mL, about 25,000 IU/mL to about 125,000 IU/mL, about 25,000 IU/mL to about 150,000 IU/mL, about 25,000 IU/mL to about 175,000 IU/mL, about 25,000 IU/mL to about 200,000 IU/mL, about 25,000 IU/mL to about 225,000 IU/mL, about 25,000 IU/mL to about 250,000 IU/mL, about 25,000 IU/mL to about 300,000 IU/mL, about 25,000 IU/mL to about 350,000 IU/mL, about 25,000 IU/mL to about 400,000 IU/mL, about 25,000 IU/mL to about 450,000 IU/mL, about 25,000 IU/mL to about 500,000 IU/mL, about 50,000 IU/mL to about 75,000 IU/mL, about 50,000 IU/mL to about 100,000 IU/mL, about 50,000 IU/mL to about 125,000 IU/mL, about 50,000 IU/mL to about 150,000 IU/mL, about 50,000 IU/mL to about 175,000 IU/mL, about 50,000 IU/mL to about 200,000 IU/mL, about 50,000 IU/mL to about 225,000 IU/mL, about 50,000 IU/mL to about 250,000 IU/mL, about 50,000 IU/mL to about 300,000 IU/mL, about 50,000 IU/mL to about 350,000 IU/mL, about 50,000 IU/mL to about 400,000 IU/mL, about 50,000 IU/mL to about 450,000 IU/mL, about 50,000 IU/mL to about 500,000 IU/mL, about 75,000 IU/mL to about 100,000 IU/mL, about 75,000 IU/mL to about 125,000 IU/mL, about 75,000 IU/mL to about 150,000 IU/mL, about 75,000 IU/mL to about 175,000 IU/mL, about 75,000 IU/mL to about 200,000 IU/mL, about 75,000 IU/mL to about 225,000 IU/mL, about 75,000 IU/mL to about 250,000 IU/mL, about 75,000 IU/mL to about 300,000 IU/mL, about 75,000 IU/mL to about 350,000 IU/mL, about 75,000 IU/mL to about 400,000 IU/mL, about 75,000 IU/mL to about 450,000 IU/mL, about 75,000 IU/mL to about 500,000 IU/mL, about 100,000 IU/mL to about 125,000 IU/mL, about 100,000 IU/mL to about 150,000 IU/mL, about 100,000 IU/mL to about 175,000 IU/mL, about 100,000 IU/mL to about 200,000 IU/mL, about 100,000 IU/mL to about 225,000 IU/mL, about 100,000 IU/mL to about 250,000 IU/mL, about 100,000 IU/mL to about 300,000 IU/mL, about 100,000 IU/mL to about 350,000 IU/mL, about 100,000 IU/mL to about 400,000 IU/mL, about 100,000 IU/mL to about 450,000 IU/mL, about 100,000 IU/mL to about 500,000 IU/mL, about 150,000 IU/mL to about 200,000 IU/mL, about 150,000 IU/mL to about 225,000 IU/mL, about 150,000 IU/mL to about 250,000 IU/mL, about 150,000 IU/mL to about 300,000 IU/mL, about 150,000 IU/mL to about 350,000 IU/mL, about 150,000 IU/mL to about 400,000 IU/mL, about 150,000 IU/mL to about 450,000 IU/mL, about 150,000 IU/mL to about 500,000 IU/mL, about 200,000 IU/mL to about 225,000 IU/mL, about 200,000 IU/mL to about 250,000 IU/mL, about 200,000 IU/mL to about 300,000 IU/mL, about 200,000 IU/mL to about 350,000 IU/mL, about 200,000 IU/mL to about 400,000 IU/mL, about 200,000 IU/mL to about 450,000 IU/mL, about 200,000 IU/mL to about 500,000 IU/mL, about 250,000 IU/mL to about 300,000 IU/mL, about 250,000 IU/mL to about 350,000 IU/mL, about 250,000 IU/mL to about 400,000 IU/mL, about 250,000 IU/mL to about 450,000 IU/mL, about 250,000 IU/mL to about 500,000 IU/mL, about 300,000 IU/mL to about 350,000 IU/mL, about 300,000 IU/mL to about 400,000 IU/mL, about 300,000 IU/mL to about 450,000 IU/mL, about 300,000 IU/mL to about 500,000 IU/mL, about 350,000 IU/mL to about 400,000 IU/mL, about 350,000 IU/mL to about 450,000 IU/mL, about 350,000 IU/mL to about 500,000 IU/mL, about 400,000 IU/mL to about 450,000 IU/mL, about 400,000 IU/mL to about 500,000 IU/mL, or about 450,000 IU/mL to about 500,000 IU/mL.

In aspects of this embodiment, a composition disclosed herein comprises a hyaluronidase in a concentration of, e.g., at least 10,000 IU/mL, at least 20,000 IU/mL, at least 30,000 IU/mL, at least 40,000 IU/mL, at least 50,000 IU/mL, at least 60,000 IU/mL, at least 70,000 IU/mL, at least 80,000 IU/mL, at least 90,000 IU/mL, or at least 100,000 IU/mL. In other aspects of this embodiment, a composition disclosed herein comprises a hyaluronidase in a concentration of, e.g., at most 10,000 IU/mL, at most 20,000 IU/mL, at most 30,000 IU/mL, at most 40,000 IU/mL, at most 50,000 IU/mL, at most 60,000 IU/mL, at most 70,000 IU/mL, at most 80,000 IU/mL, at most 90,000 IU/mL, or at most 100,000 IU/mL.

In yet other aspects of this embodiment, a composition disclosed herein comprises a hyper-concentrated hyaluronidase in a concentration of, e.g., about 10,000 IU/mL to about 20,000 IU/mL, about 10,000 IU/mL to about 30,000 IU/mL, about 10,000 IU/mL to about 40,000 IU/mL, about 10,000 IU/mL to about 50,000 IU/mL, about 10,000 IU/mL to about 60,000 IU/mL, about 10,000 IU/mL to about 70,000 IU/mL, about 10,000 IU/mL to about 80,000 IU/mL, about 10,000 IU/mL to about 90,000 IU/mL, about 10,000 IU/mL to about 100,000 IU/mL, about 20,000 IU/mL to about 30,000 IU/mL, about 20,000 IU/mL to about 40,000 IU/mL, about 20,000 IU/mL to about 50,000 IU/mL, about 20,000 IU/mL to about 60,000 IU/mL, about 20,000 IU/mL to about 70,000 IU/mL, about 20,000 IU/mL to about 80,000 IU/mL, about 20,000 IU/mL to about 90,000 IU/mL, about 20,000 IU/mL to about 100,000 IU/mL, about 30,000 IU/mL to about 40,000 IU/mL, about 30,000 IU/mL to about 50,000 IU/mL, about 30,000 IU/mL to about 60,000 IU/mL, about 30,000 IU/mL to about 70,000 IU/mL, about 30,000 IU/mL to about 80,000 IU/mL, about 30,000 IU/mL to about 90,000 IU/mL, about 30,000 IU/mL to about 100,000 IU/mL, about 40,000 IU/mL to about 50,000 IU/mL, about 40,000 IU/mL to about 60,000 IU/mL, about 40,000 IU/mL to about 70,000 IU/mL, about 40,000 IU/mL to about 80,000 IU/mL, about 40,000 IU/mL to about 90,000 IU/mL, about 40,000 IU/mL to about 100,000 IU/mL, about 50,000 IU/mL to about 60,000 IU/mL, about 50,000 IU/mL to about 70,000 IU/mL, about 50,000 IU/mL to about 80,000 IU/mL, about 50,000 IU/mL to about 90,000 IU/mL, about 50,000 IU/mL to about 100,000 IU/mL, about 60,000 IU/mL to about 70,000 IU/mL, about 60,000 IU/mL to about 80,000 IU/mL, about 60,000 IU/mL to about 90,000 IU/mL, about 60,000 IU/mL to about 100,000 IU/mL, about 70,000 IU/mL to about 80,000 IU/mL, about 70,000 IU/mL to about 90,000 IU/mL, about 70,000 IU/mL to about 100,000 IU/mL, about 80,000 IU/mL to about 90,000 IU/mL, about 80,000 IU/mL to about 100,000 IU/mL, or about 90,000 IU/mL to about 100,000 IU/mL.

In aspects of this embodiment, a composition disclosed herein comprises a hyaluronidase in a concentration of, e.g., at least 5,000 IU/mL, at least 6,000 IU/mL, at least 7,000 IU/mL, at least 8,000 IU/mL, at least 9,000 IU/mL, at least 10,000 IU/mL, at least 11,000 IU/mL, at least 12,000 IU/mL, at least 13,000 IU/mL, at least 14,000 IU/mL, at least 15,000 IU/mL, at least 16,000 IU/mL, at least 17,000 IU/mL, at least 18,000 IU/mL, at least 19,000 IU/mL, or at least 20,000 IU/mL. In other aspects of this embodiment, a composition disclosed herein comprises a hyper-concentrated hyaluronidase in a concentration of, e.g., at most 5,000 IU/mL, at most 6,000 IU/mL, at most 7,000 IU/mL, at most 8,000 IU/mL, at most 9,000 IU/mL, at most 10,000 IU/mL, at most 11,000 IU/mL, at most 12,000 IU/mL, at most 13,000 IU/mL, at most 14,000 IU/mL, at most 15,000 IU/mL, at most 16,000 IU/mL, at most 17,000 IU/mL, at most 18,000 IU/mL, at most 19,000 IU/mL, or at most 20,000 IU/mL.

In yet other aspects of this embodiment, a composition disclosed herein comprises a hyper-concentrated hyaluronidase in a concentration of, e.g., about 5,000 IU/mL to about 6,000 IU/mL, about 5,000 IU/mL to about 7,000 IU/mL, about 5,000 IU/mL to about 8,000 IU/mL, about 5,000 IU/mL to about 9,000 IU/mL, about 5,000 IU/mL to about 10,000 IU/mL, about 5,000 IU/mL to about 11,000 IU/mL, about 5,000 IU/mL to about 12,000 IU/mL, about 5,000 IU/mL to about 13,000 IU/mL, about 5,000 IU/mL to about 14,000 IU/mL, about 5,000 IU/mL to about 15,000 IU/mL, about 5,000 IU/mL to about 16,000 IU/mL, about 5,000 IU/mL to about 17,000 IU/mL, about 5,000 IU/mL to about 18,000 IU/mL, about 5,000 IU/mL to about 19,000 IU/mL, about 5,000 IU/mL to about 20,000 IU/mL, about 6,000 IU/mL to about 7,000 IU/mL, about 6,000 IU/mL to about 8,000 IU/mL, about 6,000 IU/mL to about 9,000 IU/mL, about 6,000 IU/mL to about 10,000 IU/mL, about 6,000 IU/mL to about 11,000 IU/mL, about 6,000 IU/mL to about 12,000 IU/mL, about 6,000 IU/mL to about 13,000 IU/mL, about 6,000 IU/mL to about 14,000 IU/mL, about 6,000 IU/mL to about 15,000 IU/mL, about 6,000 IU/mL to about 16,000 IU/mL, about 6,000 IU/mL to about 17,000 IU/mL, about 6,000 IU/mL to about 18,000 IU/mL, about 6,000 IU/mL to about 19,000 IU/mL, about 6,000 IU/mL to about 20,000 IU/mL, about 7,000 IU/mL to about 8,000 IU/mL, about 7,000 IU/mL to about 9,000 IU/mL, about 7,000 IU/mL to about 10,000 IU/mL, about 7,000 IU/mL to about 11,000 IU/mL, about 7,000 IU/mL to about 12,000 IU/mL, about 7,000 IU/mL to about 13,000 IU/mL, about 7,000 IU/mL to about 14,000 IU/mL, about 7,000 IU/mL to about 15,000 IU/mL, about 7,000 IU/mL to about 16,000 IU/mL, about 7,000 IU/mL to about 17,000 IU/mL, about 7,000 IU/mL to about 18,000 IU/mL, about 7,000 IU/mL to about 19,000 IU/mL, about 7,000 IU/mL to about 20,000 IU/mL, about 8,000 IU/mL to about 9,000 IU/mL, about 8,000 IU/mL to about 10,000 IU/mL, about 8,000 IU/mL to about 11,000 IU/mL, about 8,000 IU/mL to about 12,000 IU/mL, about 8,000 IU/mL to about 13,000 IU/mL, about 8,000 IU/mL to about 14,000 IU/mL, about 8,000 IU/mL to about 15,000 IU/mL, about 8,000 IU/mL to about 16,000 IU/mL, about 8,000 IU/mL to about 17,000 IU/mL, about 8,000 IU/mL to about 18,000 IU/mL, about 8,000 IU/mL to about 19,000 IU/mL, about 8,000 IU/mL to about 20,000 IU/mL, about 9,000 IU/mL to about 10,000 IU/mL, about 9,000 IU/mL to about 11,000 IU/mL, about 9,000 IU/mL to about 12,000 IU/mL, about 9,000 IU/mL to about 13,000 IU/mL, about 9,000 IU/mL to about 14,000 IU/mL, about 9,000 IU/mL to about 15,000 IU/mL, about 9,000 IU/mL to about 16,000 IU/mL, about 9,000 IU/mL to about 17,000 IU/mL, about 9,000 IU/mL to about 18,000 IU/mL, about 9,000 IU/mL to about 19,000 IU/mL, about 9,000 IU/mL to about 20,000 IU/mL, about 10,000 IU/mL to about 11,000 IU/mL, about 10,000 IU/mL to about 12,000 IU/mL, about 10,000 IU/mL to about 13,000 IU/mL, about 10,000 IU/mL to about 14,000 IU/mL, about 10,000 IU/mL to about 15,000 IU/mL, about 10,000 IU/mL to about 16,000 IU/mL, about 10,000 IU/mL to about 17,000 IU/mL, about 10,000 IU/mL to about 18,000 IU/mL, about 10,000 IU/mL to about 19,000 IU/mL, about 10,000 IU/mL to about 20,000 IU/mL, about 11,000 IU/mL to about 12,000 IU/mL, about 11,000 IU/mL to about 13,000 IU/mL, about 11,000 IU/mL to about 14,000 IU/mL, about 11,000 IU/mL to about 15,000 IU/mL, about 11,000 IU/mL to about 16,000 IU/mL, about 11,000 IU/mL to about 17,000 IU/mL, about 11,000 IU/mL to about 18,000 IU/mL, about 11,000 IU/mL to about 19,000 IU/mL, about 11,000 IU/mL to about 20,000 IU/mL, about 12,000 IU/mL to about 13,000 IU/mL, about 12,000 IU/mL to about 14,000 IU/mL, about 12,000 IU/mL to about 15,000 IU/mL, about 12,000 IU/mL to about 16,000 IU/mL, about 12,000 IU/mL to about 17,000 IU/mL, about 12,000 IU/mL to about 18,000 IU/mL, about 12,000 IU/mL to about 19,000 IU/mL, about 12,000 IU/mL to about 20,000 IU/mL, about 13,000 IU/mL to about 14,000 IU/mL, about 13,000 IU/mL to about 15,000 IU/mL, about 13,000 IU/mL to about 16,000 IU/mL, about 13,000 IU/mL to about 17,000 IU/mL, about 13,000 IU/mL to about 18,000 IU/mL, about 13,000 IU/mL to about 19,000 IU/mL, about 13,000 IU/mL to about 20,000 IU/mL, about 14,000 IU/mL to about 15,000 IU/mL, about 14,000 IU/mL to about 16,000 IU/mL, about 14,000 IU/mL to about 17,000 IU/mL, about 14,000 IU/mL to about 18,000 IU/mL, about 14,000 IU/mL to about 19,000 IU/mL, about 14,000 IU/mL to about 20,000 IU/mL, about 15,000 IU/mL to about 16,000 IU/mL, about 15,000 IU/mL to about 17,000 IU/mL, about 15,000 IU/mL to about 18,000 IU/mL, about 15,000 IU/mL to about 19,000 IU/mL, about 15,000 IU/mL to about 20,000 IU/mL, about 16,000 IU/mL to about 17,000 IU/mL, about 16,000 IU/mL to about 18,000 IU/mL, about 16,000 IU/mL to about 19,000 IU/mL, about 16,000 IU/mL to about 20,000 IU/mL, about 17,000 IU/mL to about 18,000 IU/mL, about 17,000 IU/mL to about 19,000 IU/mL, about 17,000 IU/mL to about 20,000 IU/mL, about 18,000 IU/mL to about 19,000 IU/mL, about 18,000 IU/mL to about 20,000 IU/mL, or about 19,000 IU/mL to about 20,000 IU/mL.

In aspects of this embodiment, a composition disclosed herein comprises a hyaluronidase in a concentration of, e.g., at least 250 IU/mL, at least 500 IU/mL, at least 750 IU/mL, at least 1,000 IU/mL, at least 1,500 IU/mL, at least 2,000 IU/mL, at least 2,500 IU/mL, at least 3,000 IU/mL, at least 3,500 IU/mL, at least 4,000 IU/mL, at least 4,500 IU/mL, or at least 5,000 IU/mL. In other aspects of this embodiment, a composition disclosed herein comprises a hyper-concentrated hyaluronidase in a concentration of, e.g., at most 250 IU/mL, at most 500 IU/mL, at most 750 IU/mL, at most 1,000 IU/mL, at most 1,500 IU/mL, at most 2,000 IU/mL, at most 2,500 IU/mL, at most 3,000 IU/mL, at most 3,500 IU/mL, at most 4,000 IU/mL, at most 4,500 IU/mL, or at most 5,000 IU/mL.

In yet other aspects of this embodiment, a composition disclosed herein comprises a hyper-concentrated hyaluronidase in a concentration of, e.g., about 250 IU/mL to about 500 IU/mL, about 250 IU/mL to about 750 IU/mL, about 250 IU/mL to about 1,000 IU/mL, about 250 IU/mL to about 1,500 IU/mL, about 250 IU/mL to about 2,000 IU/mL, about 250 IU/mL to about 2,500 IU/mL, about 250 IU/mL to about 3,000 IU/mL, about 250 IU/mL to about 3,500 IU/mL, about 250 IU/mL to about 4,000 IU/mL, about 250 IU/mL to about 4,500 IU/mL, about 250 IU/mL to about 5,000 IU/mL, about 500 IU/mL to about 750 IU/mL, about 500 IU/mL to about 1,000 IU/mL, about 500 IU/mL to about 1,500 IU/mL, about 500 IU/mL to about 2,000 IU/mL, about 500 IU/mL to about 2,500 IU/mL, about 500 IU/mL to about 3,000 IU/mL, about 500 IU/mL to about 3,500 IU/mL, about 500 IU/mL to about 4,000 IU/mL, about 500 IU/mL to about 4,500 IU/mL, about 500 IU/mL to about 5,000 IU/mL, about 750 IU/mL to about 1,000 IU/mL, about 750 IU/mL to about 1,500 IU/mL, about 750 IU/mL to about 2,000 IU/mL, about 750 IU/mL to about 2,500 IU/mL, about 750 IU/mL to about 3,000 IU/mL, about 750 IU/mL to about 3,500 IU/mL, about 750 IU/mL to about 4,000 IU/mL, about 750 IU/mL to about 4,500 IU/mL, about 750 IU/mL to about 5,000 IU/mL, about 1,000 IU/mL to about 1,500 IU/mL, about 1,000 IU/mL to about 2,000 IU/mL, about 1,000 IU/mL to about 2,500 IU/mL, about 1,000 IU/mL to about 3,000 IU/mL, about 1,000 IU/mL to about 3,500 IU/mL, about 1,000 IU/mL to about 4,000 IU/mL, about 1,000 IU/mL to about 4,500 IU/mL, about 1,000 IU/mL to about 5,000 IU/mL, about 1,500 IU/mL to about 2,000 IU/mL, about 1,500 IU/mL to about 2,500 IU/mL, about 1,500 IU/mL to about 3,000 IU/mL, about 1,500 IU/mL to about 3,500 IU/mL, about 1,500 IU/mL to about 4,000 IU/mL, about 1,500 IU/mL to about 4,500 IU/mL, about 1,500 IU/mL to about 5,000 IU/mL, about 2,000 IU/mL to about 2,500 IU/mL, about 2,000 IU/mL to about 3,000 IU/mL, about 2,000 IU/mL to about 3,500 IU/mL, about 2,000 IU/mL to about 4,000 IU/mL, about 2,000 IU/mL to about 4,500 IU/mL, about 2,000 IU/mL to about 5,000 IU/mL, about 2,500 IU/mL to about 3,000 IU/mL, about 2,500 IU/mL to about 3,500 IU/mL, about 2,500 IU/mL to about 4,000 IU/mL, about 2,500 IU/mL to about 4,500 IU/mL, about 2,500 IU/mL to about 5,000 IU/mL, about 3,000 IU/mL to about 3,500 IU/mL, about 3,000 IU/mL to about 4,000 IU/mL, about 3,000 IU/mL to about 4,500 IU/mL, about 3,000 IU/mL to about 5,000 IU/mL, about 3,500 IU/mL to about 4,000 IU/mL, about 3,500 IU/mL to about 4,500 IU/mL, about 3,500 IU/mL to about 5,000 IU/mL, about 4,000 IU/mL to about 4,500 IU/mL, about 4,000 IU/mL to about 5,000 IU/mL, or about 4,500 IU/mL to about 5,000 IU/mL.

A single-dose amount of a composition comprising a hyper-concentrated hyaluronidase used to effectively reduce or eliminate a hyaluronic acid-induced blockage in one or more blood vessels of an area of tissue being affected by the blockage can include between about 250 IU/mL and about 500,000 IU/mL of a hyaluronidase. In aspects of this embodiment, a single-dose amount of a composition comprising a hyaluronidase used to effectively reduce or eliminate a hyaluronic acid-induced blockage in one or more blood vessels of an area of tissue being affected by the blockage can include a hyaluronidase in an amount of, e.g., at least 25,000 IU/mL, at least 50,000 IU/mL, at least 75,000 IU/mL, at least 100,000 IU/mL, at least 125,000 IU/mL, at least 150,000 IU/mL, at least 175,000 IU/mL, at least 200,000 IU/mL, at least 225,000 IU/mL, at least 250,000 IU/mL, at least 300,000 IU/mL, at least 350,000 IU/mL, at least 400,000 IU/mL, at least 450,000 IU/mL, or at least 500,000 IU/mL. In other aspects of this embodiment, a single-dose amount of a composition comprising a hyaluronidase used to effectively reduce or eliminate a hyaluronic acid-induced blockage in one or more blood vessels of an area of tissue being affected by the blockage can include a hyaluronidase in an amount of, e.g., at most 25,000 IU/mL, at most 50,000 IU/mL, at most 75,000 IU/mL, at most 100,000 IU/mL, at most 125,000 IU/mL, at most 150,000 IU/mL, at most 175,000 IU/mL, at most 200,000 IU/mL, at most 225,000 IU/mL, at most 250,000 IU/mL, at most 300,000 IU/mL, at most 350,000 IU/mL, at most 400,000 IU/mL, at most 450,000 IU/mL, or at most 500,000 IU/mL.

In yet other aspects of this embodiment, a single-dose amount of a composition comprising a hyaluronidase used to effectively reduce or eliminate a hyaluronic acid-induced blockage in one or more blood vessels of an area of tissue being affected by the blockage can include a hyaluronidase in an amount of, e.g., about 25,000 IU/mL to about 50,000 IU/mL, about 25,000 IU/mL to about 75,000 IU/mL, about 25,000 IU/mL to about 100,000 IU/mL, about 25,000 IU/mL to about 125,000 IU/mL, about 25,000 IU/mL to about 150,000 IU/mL, about 25,000 IU/mL to about 175,000 IU/mL, about 25,000 IU/mL to about 200,000 IU/mL, about 25,000 IU/mL to about 225,000 IU/mL, about 25,000 IU/mL to about 250,000 IU/mL, about 25,000 IU/mL to about 300,000 IU/mL, about 25,000 IU/mL to about 350,000 IU/mL, about 25,000 IU/mL to about 400,000 IU/mL, about 25,000 IU/mL to about 450,000 IU/mL, about 25,000 IU/mL to about 500,000 IU/mL, about 50,000 IU/mL to about 75,000 IU/mL, about 50,000 IU/mL to about 100,000 IU/mL, about 50,000 IU/mL to about 125,000 IU/mL, about 50,000 IU/mL to about 150,000 IU/mL, about 50,000 IU/mL to about 175,000 IU/mL, about 50,000 IU/mL to about 200,000 IU/mL, about 50,000 IU/mL to about 225,000 IU/mL, about 50,000 IU/mL to about 250,000 IU/mL, about 50,000 IU/mL to about 300,000 IU/mL, about 50,000 IU/mL to about 350,000 IU/mL, about 50,000 IU/mL to about 400,000 IU/mL, about 50,000 IU/mL to about 450,000 IU/mL, about 50,000 IU/mL to about 500,000 IU/mL, about 75,000 IU/mL to about 100,000 IU/mL, about 75,000 IU/mL to about 125,000 IU/mL, about 75,000 IU/mL to about 150,000 IU/mL, about 75,000 IU/mL to about 175,000 IU/mL, about 75,000 IU/mL to about 200,000 IU/mL, about 75,000 IU/mL to about 225,000 IU/mL, about 75,000 IU/mL to about 250,000 IU/mL, about 75,000 IU/mL to about 300,000 IU/mL, about 75,000 IU/mL to about 350,000 IU/mL, about 75,000 IU/mL to about 400,000 IU/mL, about 75,000 IU/mL to about 450,000 IU/mL, about 75,000 IU/mL to about 500,000 IU/mL, about 100,000 IU/mL to about 125,000 IU/mL, about 100,000 IU/mL to about 150,000 IU/mL, about 100,000 IU/mL to about 175,000 IU/mL, about 100,000 IU/mL to about 200,000 IU/mL, about 100,000 IU/mL to about 225,000 IU/mL, about 100,000 IU/mL to about 250,000 IU/mL, about 100,000 IU/mL to about 300,000 IU/mL, about 100,000 IU/mL to about 350,000 IU/mL, about 100,000 IU/mL to about 400,000 IU/mL, about 100,000 IU/mL to about 450,000 IU/mL, about 100,000 IU/mL to about 500,000 IU/mL, about 150,000 IU/mL to about 200,000 IU/mL, about 150,000 IU/mL to about 225,000 IU/mL, about 150,000 IU/mL to about 250,000 IU/mL, about 150,000 IU/mL to about 300,000 IU/mL, about 150,000 IU/mL to about 350,000 IU/mL, about 150,000 IU/mL to about 400,000 IU/mL, about 150,000 IU/mL to about 450,000 IU/mL, about 150,000 IU/mL to about 500,000 IU/mL, about 200,000 IU/mL to about 225,000 IU/mL, about 200,000 IU/mL to about 250,000 IU/mL, about 200,000 IU/mL to about 300,000 IU/mL, about 200,000 IU/mL to about 350,000 IU/mL, about 200,000 IU/mL to about 400,000 IU/mL, about 200,000 IU/mL to about 450,000 IU/mL, about 200,000 IU/mL to about 500,000 IU/mL, about 250,000 IU/mL to about 300,000 IU/mL, about 250,000 IU/mL to about 350,000 IU/mL, about 250,000 IU/mL to about 400,000 IU/mL, about 250,000 IU/mL to about 450,000 IU/mL, about 250,000 IU/mL to about 500,000 IU/mL, about 300,000 IU/mL to about 350,000 IU/mL, about 300,000 IU/mL to about 400,000 IU/mL, about 300,000 IU/mL to about 450,000 IU/mL, about 300,000 IU/mL to about 500,000 IU/mL, about 350,000 IU/mL to about 400,000 IU/mL, about 350,000 IU/mL to about 450,000 IU/mL, about 350,000 IU/mL to about 500,000 IU/mL, about 400,000 IU/mL to about 450,000 IU/mL, about 400,000 IU/mL to about 500,000 IU/mL, or about 450,000 IU/mL to about 500,000 IU/mL.

In aspects of this embodiment, a single-dose amount of a composition comprising a hyaluronidase used to effectively reduce or eliminate a hyaluronic acid-induced blockage in one or more blood vessels of an area of tissue being affected by the blockage can include a hyaluronidase in an amount of, e.g., at least 10,000 IU/mL, at least 20,000 IU/mL, at least 30,000 IU/mL, at least 40,000 IU/mL, at least 50,000 IU/mL, at least 60,000 IU/mL, at least 70,000 IU/mL, at least 80,000 IU/mL, at least 90,000 IU/mL, or at least 100,000 IU/mL. In other aspects of this embodiment, a single-dose amount of a composition comprising a hyaluronidase used to effectively reduce or eliminate a hyaluronic acid-induced blockage in one or more blood vessels of an area of tissue being affected by the blockage can include a hyaluronidase in an amount of, e.g., at most 10,000 IU/mL, at most 20,000 IU/mL, at most 30,000 IU/mL, at most 40,000 IU/mL, at most 50,000 IU/mL, at most 60,000 IU/mL, at most 70,000 IU/mL, at most 80,000 IU/mL, at most 90,000 IU/mL, or at most 100,000 IU/mL.

In yet other aspects of this embodiment, a single-dose amount of a composition comprising a hyaluronidase used to effectively reduce or eliminate a hyaluronic acid-induced blockage in one or more blood vessels of an area of tissue being affected by the blockage can include a hyaluronidase in an amount of, e.g., about 10,000 IU/mL to about 20,000 IU/mL, about 10,000 IU/mL to about 30,000 IU/mL, about 10,000 IU/mL to about 40,000 IU/mL, about 10,000 IU/mL to about 50,000 IU/mL, about 10,000 IU/mL to about 60,000 IU/mL, about 10,000 IU/mL to about 70,000 IU/mL, about 10,000 IU/mL to about 80,000 IU/mL, about 10,000 IU/mL to about 90,000 IU/mL, about 10,000 IU/mL to about 100,000 IU/mL, about 20,000 IU/mL to about 30,000 IU/mL, about 20,000 IU/mL to about 40,000 IU/mL, about 20,000 IU/mL to about 50,000 IU/mL, about 20,000 IU/mL to about 60,000 IU/mL, about 20,000 IU/mL to about 70,000 IU/mL, about 20,000 IU/mL to about 80,000 IU/mL, about 20,000 IU/mL to about 90,000 IU/mL, about 20,000 IU/mL to about 100,000 IU/mL, about 30,000 IU/mL to about 40,000 IU/mL, about 30,000 IU/mL to about 50,000 IU/mL, about 30,000 IU/mL to about 60,000 IU/mL, about 30,000 IU/mL to about 70,000 IU/mL, about 30,000 IU/mL to about 80,000 IU/mL, about 30,000 IU/mL to about 90,000 IU/mL, about 30,000 IU/mL to about 100,000 IU/mL, about 40,000 IU/mL to about 50,000 IU/mL, about 40,000 IU/mL to about 60,000 IU/mL, about 40,000 IU/mL to about 70,000 IU/mL, about 40,000 IU/mL to about 80,000 IU/mL, about 40,000 IU/mL to about 90,000 IU/mL, about 40,000 IU/mL to about 100,000 IU/mL, about 50,000 IU/mL to about 60,000 IU/mL, about 50,000 IU/mL to about 70,000 IU/mL, about 50,000 IU/mL to about 80,000 IU/mL, about 50,000 IU/mL to about 90,000 IU/mL, about 50,000 IU/mL to about 100,000 IU/mL, about 60,000 IU/mL to about 70,000 IU/mL, about 60,000 IU/mL to about 80,000 IU/mL, about 60,000 IU/mL to about 90,000 IU/mL, about 60,000 IU/mL to about 100,000 IU/mL, about 70,000 IU/mL to about 80,000 IU/mL, about 70,000 IU/mL to about 90,000 IU/mL, about 70,000 IU/mL to about 100,000 IU/mL, about 80,000 IU/mL to about 90,000 IU/mL, about 80,000 IU/mL to about 100,000 IU/mL, or about 90,000 IU/mL to about 100,000 IU/mL.

In aspects of this embodiment, a single-dose amount of a composition comprising a hyaluronidase used to effectively reduce or eliminate a hyaluronic acid-induced blockage in one or more blood vessels of an area of tissue being affected by the blockage can include a hyaluronidase in an amount of, e.g., at least 5,000 IU/mL, at least 6,000 IU/mL, at least 7,000 IU/mL, at least 8,000 IU/mL, at least 9,000 IU/mL, at least 10,000 IU/mL, at least 11,000 IU/mL, at least 12,000 IU/mL, at least 13,000 IU/mL, at least 14,000 IU/mL, at least 15,000 IU/mL, at least 16,000 IU/mL, at least 17,000 IU/mL, at least 18,000 IU/mL, at least 19,000 IU/mL, or at least 20,000 IU/mL. In other aspects of this embodiment, a single-dose amount of a composition comprising a hyaluronidase used to effectively reduce or eliminate a hyaluronic acid-induced blockage in one or more blood vessels of an area of tissue being affected by the blockage can include a hyaluronidase in an amount of, e.g., at most 5,000 IU/mL, at most 6,000 IU/mL, at most 7,000 IU/mL, at most 8,000 IU/mL, at most 9,000 IU/mL, at most 10,000 IU/mL, at most 11,000 IU/mL, at most 12,000 IU/mL, at most 13,000 IU/mL, at most 14,000 IU/mL, at most 15,000 IU/mL, at most 16,000 IU/mL, at most 17,000 IU/mL, at most 18,000 IU/mL, at most 19,000 IU/mL, or at most 20,000 IU/mL.

In yet other aspects of this embodiment, a single-dose amount of a composition comprising a hyaluronidase used to effectively reduce or eliminate a hyaluronic acid-induced blockage in one or more blood vessels of an area of tissue being affected by the blockage can include a hyaluronidase in an amount of, e.g., about 5,000 IU/mL to about 6,000 IU/mL, about 5,000 IU/mL to about 7,000 IU/mL, about 5,000 IU/mL to about 8,000 IU/mL, about 5,000 IU/mL to about 9,000 IU/mL, about 5,000 IU/mL to about 10,000 IU/mL, about 5,000 IU/mL to about 11,000 IU/mL, about 5,000 IU/mL to about 12,000 IU/mL, about 5,000 IU/mL to about 13,000 IU/mL, about 5,000 IU/mL to about 14,000 IU/mL, about 5,000 IU/mL to about 15,000 IU/mL, about 5,000 IU/mL to about 16,000 IU/mL, about 5,000 IU/mL to about 17,000 IU/mL, about 5,000 IU/mL to about 18,000 IU/mL, about 5,000 IU/mL to about 19,000 IU/mL, about 5,000 IU/mL to about 20,000 IU/mL, about 6,000 IU/mL to about 7,000 IU/mL, about 6,000 IU/mL to about 8,000 IU/mL, about 6,000 IU/mL to about 9,000 IU/mL, about 6,000 IU/mL to about 10,000 IU/mL, about 6,000 IU/mL to about 11,000 IU/mL, about 6,000 IU/mL to about 12,000 IU/mL, about 6,000 IU/mL to about 13,000 IU/mL, about 6,000 IU/mL to about 14,000 IU/mL, about 6,000 IU/mL to about 15,000 IU/mL, about 6,000 IU/mL to about 16,000 IU/mL, about 6,000 IU/mL to about 17,000 IU/mL, about 6,000 IU/mL to about 18,000 IU/mL, about 6,000 IU/mL to about 19,000 IU/mL, about 6,000 IU/mL to about 20,000 IU/mL, about 7,000 IU/mL to about 8,000 IU/mL, about 7,000 IU/mL to about 9,000 IU/mL, about 7,000 IU/mL to about 10,000 IU/mL, about 7,000 IU/mL to about 11,000 IU/mL, about 7,000 IU/mL to about 12,000 IU/mL, about 7,000 IU/mL to about 13,000 IU/mL, about 7,000 IU/mL to about 14,000 IU/mL, about 7,000 IU/mL to about 15,000 IU/mL, about 7,000 IU/mL to about 16,000 IU/mL, about 7,000 IU/mL to about 17,000 IU/mL, about 7,000 IU/mL to about 18,000 IU/mL, about 7,000 IU/mL to about 19,000 IU/mL, about 7,000 IU/mL to about 20,000 IU/mL, about 8,000 IU/mL to about 9,000 IU/mL, about 8,000 IU/mL to about 10,000 IU/mL, about 8,000 IU/mL to about 11,000 IU/mL, about 8,000 IU/mL to about 12,000 IU/mL, about 8,000 IU/mL to about 13,000 IU/mL, about 8,000 IU/mL to about 14,000 IU/mL, about 8,000 IU/mL to about 15,000 IU/mL, about 8,000 IU/mL to about 16,000 IU/mL, about 8,000 IU/mL to about 17,000 IU/mL, about 8,000 IU/mL to about 18,000 IU/mL, about 8,000 IU/mL to about 19,000 IU/mL, about 8,000 IU/mL to about 20,000 IU/mL, about 9,000 IU/mL to about 10,000 IU/mL, about 9,000 IU/mL to about 11,000 IU/mL, about 9,000 IU/mL to about 12,000 IU/mL, about 9,000 IU/mL to about 13,000 IU/mL, about 9,000 IU/mL to about 14,000 IU/mL, about 9,000 IU/mL to about 15,000 IU/mL, about 9,000 IU/mL to about 16,000 IU/mL, about 9,000 IU/mL to about 17,000 IU/mL, about 9,000 IU/mL to about 18,000 IU/mL, about 9,000 IU/mL to about 19,000 IU/mL, about 9,000 IU/mL to about 20,000 IU/mL, about 10,000 IU/mL to about 11,000 IU/mL, about 10,000 IU/mL to about 12,000 IU/mL, about 10,000 IU/mL to about 13,000 IU/mL, about 10,000 IU/mL to about 14,000 IU/mL, about 10,000 IU/mL to about 15,000 IU/mL, about 10,000 IU/mL to about 16,000 IU/mL, about 10,000 IU/mL to about 17,000 IU/mL, about 10,000 IU/mL to about 18,000 IU/mL, about 10,000 IU/mL to about 19,000 IU/mL, about 10,000 IU/mL to about 20,000 IU/mL, about 11,000 IU/mL to about 12,000 IU/mL, about 11,000 IU/mL to about 13,000 IU/mL, about 11,000 IU/mL to about 14,000 IU/mL, about 11,000 IU/mL to about 15,000 IU/mL, about 11,000 IU/mL to about 16,000 IU/mL, about 11,000 IU/mL to about 17,000 IU/mL, about 11,000 IU/mL to about 18,000 IU/mL, about 11,000 IU/mL to about 19,000 IU/mL, about 11,000 IU/mL to about 20,000 IU/mL, about 12,000 IU/mL to about 13,000 IU/mL, about 12,000 IU/mL to about 14,000 IU/mL, about 12,000 IU/mL to about 15,000 IU/mL, about 12,000 IU/mL to about 16,000 IU/mL, about 12,000 IU/mL to about 17,000 IU/mL, about 12,000 IU/mL to about 18,000 IU/mL, about 12,000 IU/mL to about 19,000 IU/mL, about 12,000 IU/mL to about 20,000 IU/mL, about 13,000 IU/mL to about 14,000 IU/mL, about 13,000 IU/mL to about 15,000 IU/mL, about 13,000 IU/mL to about 16,000 IU/mL, about 13,000 IU/mL to about 17,000 IU/mL, about 13,000 IU/mL to about 18,000 IU/mL, about 13,000 IU/mL to about 19,000 IU/mL, about 13,000 IU/mL to about 20,000 IU/mL, about 14,000 IU/mL to about 15,000 IU/mL, about 14,000 IU/mL to about 16,000 IU/mL, about 14,000 IU/mL to about 17,000 IU/mL, about 14,000 IU/mL to about 18,000 IU/mL, about 14,000 IU/mL to about 19,000 IU/mL, about 14,000 IU/mL to about 20,000 IU/mL, about 15,000 IU/mL to about 16,000 IU/mL, about 15,000 IU/mL to about 17,000 IU/mL, about 15,000 IU/mL to about 18,000 IU/mL, about 15,000 IU/mL to about 19,000 IU/mL, about 15,000 IU/mL to about 20,000 IU/mL, about 16,000 IU/mL to about 17,000 IU/mL, about 16,000 IU/mL to about 18,000 IU/mL, about 16,000 IU/mL to about 19,000 IU/mL, about 16,000 IU/mL to about 20,000 IU/mL, about 17,000 IU/mL to about 18,000 IU/mL, about 17,000 IU/mL to about 19,000 IU/mL, about 17,000 IU/mL to about 20,000 IU/mL, about 18,000 IU/mL to about 19,000 IU/mL, about 18,000 IU/mL to about 20,000 IU/mL, or about 19,000 IU/mL to about 20,000 IU/mL.

In aspects of this embodiment, a single-dose amount of a composition comprising a hyaluronidase used to effectively reduce or eliminate a hyaluronic acid-induced blockage in one or more blood vessels of an area of tissue being affected by the blockage can include a hyaluronidase in an amount of, e.g., at least 250 IU/mL, at least 500 IU/mL, at least 750 IU/mL, at least 1,000 IU/mL, at least 1,500 IU/mL, at least 2,000 IU/mL, at least 2,500 IU/mL, at least 3,000 IU/mL, at least 3,500 IU/mL, at least 4,000 IU/mL, at least 4,500 IU/mL, or at least 5,000 IU/mL. In other aspects of this embodiment, a single-dose amount of a composition comprising a hyaluronidase used to effectively reduce or eliminate a hyaluronic acid-induced blockage in one or more blood vessels of an area of tissue being affected by the blockage can include a hyaluronidase in an amount of, e.g., at most 250 IU/mL, at most 500 IU/mL, at most 750 IU/mL, at most 1,000 IU/mL, at most 1,500 IU/mL, at most 2,000 IU/mL, at most 2,500 IU/mL, at most 3,000 IU/mL, at most 3,500 IU/mL, at most 4,000 IU/mL, at most 4,500 IU/mL, or at most 5,000 IU/mL.

In yet other aspects of this embodiment, a single-dose amount of a composition comprising a hyaluronidase used to effectively reduce or eliminate a hyaluronic acid-induced blockage in one or more blood vessels of an area of tissue being affected by the blockage can include a hyaluronidase in an amount of, e.g., about 250 IU/mL to about 500 IU/mL, about 250 IU/mL to about 750 IU/mL, about 250 IU/mL to about 1,000 IU/mL, about 250 IU/mL to about 1,500 IU/mL, about 250 IU/mL to about 2,000 IU/mL, about 250 IU/mL to about 2,500 IU/mL, about 250 IU/mL to about 3,000 IU/mL, about 250 IU/mL to about 3,500 IU/mL, about 250 IU/mL to about 4,000 IU/mL, about 250 IU/mL to about 4,500 IU/mL, about 250 IU/mL to about 5,000 IU/mL, about 500 IU/mL to about 750 IU/mL, about 500 IU/mL to about 1,000 IU/mL, about 500 IU/mL to about 1,500 IU/mL, about 500 IU/mL to about 2,000 IU/mL, about 500 IU/mL to about 2,500 IU/mL, about 500 IU/mL to about 3,000 IU/mL, about 500 IU/mL to about 3,500 IU/mL, about 500 IU/mL to about 4,000 IU/mL, about 500 IU/mL to about 4,500 IU/mL, about 500 IU/mL to about 5,000 IU/mL, about 750 IU/mL to about 1,000 IU/mL, about 750 IU/mL to about 1,500 IU/mL, about 750 IU/mL to about 2,000 IU/mL, about 750 IU/mL to about 2,500 IU/mL, about 750 IU/mL to about 3,000 IU/mL, about 750 IU/mL to about 3,500 IU/mL, about 750 IU/mL to about 4,000 IU/mL, about 750 IU/mL to about 4,500 IU/mL, about 750 IU/mL to about 5,000 IU/mL, about 1,000 IU/mL to about 1,500 IU/mL, about 1,000 IU/mL to about 2,000 IU/mL, about 1,000 IU/mL to about 2,500 IU/mL, about 1,000 IU/mL to about 3,000 IU/mL, about 1,000 IU/mL to about 3,500 IU/mL, about 1,000 IU/mL to about 4,000 IU/mL, about 1,000 IU/mL to about 4,500 IU/mL, about 1,000 IU/mL to about 5,000 IU/mL, about 1,500 IU/mL to about 2,000 IU/mL, about 1,500 IU/mL to about 2,500 IU/mL, about 1,500 IU/mL to about 3,000 IU/mL, about 1,500 IU/mL to about 3,500 IU/mL, about 1,500 IU/mL to about 4,000 IU/mL, about 1,500 IU/mL to about 4,500 IU/mL, about 1,500 IU/mL to about 5,000 IU/mL, about 2,000 IU/mL to about 2,500 IU/mL, about 2,000 IU/mL to about 3,000 IU/mL, about 2,000 IU/mL to about 3,500 IU/mL, about 2,000 IU/mL to about 4,000 IU/mL, about 2,000 IU/mL to about 4,500 IU/mL, about 2,000 IU/mL to about 5,000 IU/mL, about 2,500 IU/mL to about 3,000 IU/mL, about 2,500 IU/mL to about 3,500 IU/mL, about 2,500 IU/mL to about 4,000 IU/mL, about 2,500 IU/mL to about 4,500 IU/mL, about 2,500 IU/mL to about 5,000 IU/mL, about 3,000 IU/mL to about 3,500 IU/mL, about 3,000 IU/mL to about 4,000 IU/mL, about 3,000 IU/mL to about 4,500 IU/mL, about 3,000 IU/mL to about 5,000 IU/mL, about 3,500 IU/mL to about 4,000 IU/mL, about 3,500 IU/mL to about 4,500 IU/mL, about 3,500 IU/mL to about 5,000 IU/mL, about 4,000 IU/mL to about 4,500 IU/mL, about 4,000 IU/mL to about 5,000 IU/mL, or about 4,500 IU/mL to about 5,000 IU/mL.

With respect to a multi-dose administration of a composition comprising a hyper-concentrated hyaluronidase disclosed herein, the administration of all doses should occur quickly in order to be as effective as possible in reducing or eliminating a hyaluronic acid-induced blockage in one or more blood vessels of an area of tissue being affected by the blockage. In aspects of this embodiment, all doses of a multi-dose administration occur within, e.g., at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 11 minutes, at least 12 minutes, at least 13 minutes, at least 14 minutes, at least 15 minutes, at least 30 minutes, at least 45 minutes, at least 60 minutes, at least 75 minutes or at least 90 minutes. In other aspects of this embodiment, all doses of a multi-dose administration occur within, e.g., at most 1 minute, at most 2 minutes, at most 3 minutes, at most 4 minutes, at most 5 minutes, at most 6 minutes, at most 7 minutes, at most 8 minutes, at most 9 minutes, at most 10 minutes, at most 11 minutes, at most 12 minutes, at most 13 minutes, at most 14 minutes, at most 15 minutes, at most 30 minutes, at most 45 minutes, at most 60 minutes, at most 75 minutes or at most 90 minutes. In yet other aspects of this embodiment, all doses of a multi-dose administration occur within, e.g., about 1 to about 2 minutes, about 1 to about 3 minutes, about 1 to about 4 minutes, about 1 to about 5 minutes, about 1 to about 6 minutes, about 1 to about 10 minutes, about 1 to about 15 minutes, about 1 to about 30 minutes, about 1 to about 45 minutes, about 1 to about 60 minutes, about 1 to about 75 minutes, about 1 to about 90 minutes, about 2 to about 3 minutes, about 2 to about 4 minutes, about 2 to about 5 minutes, about 2 to about 6 minutes, about 2 to about 10 minutes, about 2 to about 15 minutes, about 2 to about 30 minutes, about 2 to about 45 minutes, about 2 to about 60 minutes, about 2 to about 75 minutes, about 2 to about 90 minutes, about 3 to about 4 minutes, about 3 to about 5 minutes, about 3 to about 6 minutes, about 3 to about 10 minutes, about 3 to about 15 minutes, about 3 to about 30 minutes, about 3 to about 45 minutes, about 3 to about 60 minutes, about 3 to about 75 minutes, about 3 to about 90 minutes, about 4 to about 5 minutes, about 4 to about 6 minutes, about 4 to about 10 minutes, about 4 to about 15 minutes, about 4 to about 30 minutes, about 4 to about 45 minutes, about 4 to about 60 minutes, about 4 to about 75 minutes, about 4 to about 90 minutes, about 5 to about 6 minutes, about 5 to about 7 minutes, about 5 to about 8 minutes, about 5 to about 9 minutes, about 5 to about 10 minutes, about 5 to about 11 minutes, about 5 to about 15 minutes, about 5 to about 30 minutes, about 5 to about 45 minutes, about 5 to about 60 minutes, about 5 to about 75 minutes, about 5 to about 90 minutes, about 6 to about 7 minutes, about 6 to about 8 minutes, about 6 to about 9 minutes, about 6 to about 10 minutes, about 6 to about 11 minutes, about 6 to about 15 minutes, about 6 to about 30 minutes, about 6 to about 45 minutes, about 6 to about 60 minutes, about 6 to about 75 minutes, about 6 to about 90 minutes, about 7 to about 8 minutes, about 7 to about 9 minutes, about 7 to about 10 minutes, about 7 to about 11 minutes, about 7 to about 15 minutes, about 7 to about 30 minutes, about 7 to about 45 minutes, about 7 to about 60 minutes, about 7 to about 75 minutes, about 7 to about 90 minutes, about 8 to about 9 minutes, about 8 to about 10 minutes, about 8 to about 11 minutes, about 8 to about 15 minutes, about 8 to about 30 minutes, about 8 to about 45 minutes, about 8 to about 60 minutes, about 8 to about 75 minutes, about 8 to about 90 minutes, about 9 to about 10 minutes, about 9 to about 11 minutes, about 9 to about 15 minutes, about 9 to about 30 minutes, about 9 to about 45 minutes, about 9 to about 60 minutes, about 9 to about 75 minutes, about 9 to about 90 minutes, about 10 to about 11 minutes, about 10 to about 12 minutes, about 10 to about 13 minutes, about 10 to about 14 minutes, about 10 to about 15 minutes, about 10 to about 30 minutes, about 10 to about 45 minutes, about 10 to about 60 minutes, about 10 to about 75 minutes, about 10 to about 90 minutes, about 11 to about 12 minutes, about 11 to about 13 minutes, about 11 to about 14 minutes, about 11 to about 15 minutes, about 11 to about 30 minutes, about 11 to about 45 minutes, about 11 to about 60 minutes, about 11 to about 75 minutes, about 11 to about 90 minutes, about 12 to about 13 minutes, about 12 to about 14 minutes, about 12 to about 15 minutes, about 12 to about 30 minutes, about 12 to about 45 minutes, about 12 to about 60 minutes, about 12 to about 75 minutes, about 12 to about 90 minutes, about 13 to about 14 minutes, about 13 to about 15 minutes, about 14 to about 15 minutes about 14 to about 30 minutes, about 14 to about 45 minutes, about 14 to about 60 minutes, about 14 to about 75 minutes, about 14 to about 90 minutes, about 15 to about 30 minutes, about 15 to about 45 minutes, about 15 to about 60 minutes, about 15 to about 75 minutes, about 15 to about 90 minutes, about 30 to about 45 minutes, about 30 to about 60 minutes, about 30 to about 75 minutes, about 30 to about 90 minutes, about 45 to about 60 minutes, about 45 to about 75 minutes, about 45 to about 90 minutes, about 60 to about 75 minutes, about 60 to about 90 minutes, or about 75 to about 90 minutes. In still other aspects of this embodiment, the number of doses administered in a multi-dose administration is from, e.g., about 2 to about 5 minutes, about 2 to about 10 minutes, about 2 to about 15 minutes, about 5 to about 10 minutes, about 5 to about 15 minutes, about 5 minutes to about 30 minutes, about 10 minutes to about 15 minutes, about 10 minutes to about 30 minutes, about 15 minutes to about 30 minutes, about 15 minutes to about 45 minutes, about 15 minutes to about 60 minutes, about 15 minutes to about 75 minutes, or about 15 minutes to about 90 minutes.

With respect to a multi-dose administration of a composition comprising a hyper-concentrated hyaluronidase disclosed herein, the number of doses administered is the number needed to achieve an effective amount of hyaluronidase in order to reduce or eliminate a hyaluronic acid-induced blockage in one or more blood vessels of an area of tissue being affected by the blockage. In aspects of this embodiment, the number of doses administered in a multi-dose administration is, e.g., at least 2, at least 3, at least 4, at least 5, at least 6, at least 7, at least 8, at least 9, at least 10, at least 11, at least 12, at least 13, at least 14, or at least 15. In other aspects of this embodiment, the number of doses administered in a multi-dose administration is, e.g., at most 2, at most 3, at most 4, at most 5, at most 6, at most 7, at most 8, at most 9, at most 10, at most 11, at most 12, at most 13, at most 14, or at most 15. In yet other aspects of this embodiment, the number of doses administered in a multi-dose administration is from, e.g., about 2 to about 3, about 2 to about 4, about 2 to about 5, about 2 to about 6, about 3 to about 4, about 3 to about 5, about 3 to about 6, about 4 to about 5, about 4 to about 6, about 5 to about 6, about 5 to about 7, about 5 to about 8, about 5 to about 9, about 5 to about 10, about 5 to about 11, about 6 to about 7, about 6 to about 8, about 6 to about 9, about 6 to about 10, about 6 to about 11, about 7 to about 8, about 7 to about 9, about 7 to about 10, about 7 to about 11, about 8 to about 9, about 8 to about 10, about 8 to about 11, about 9 to about 10, about 9 to about 11, about 10 to about 11, about 10 to about 12, about 10 to about 13, about 10 to about 14, about 10 to about 15, about 11 to about 12, about 11 to about 13, about 11 to about 14, about 11 to about 15, about 12 to about 13, about 12 to about 14, about 12 to about 15, about 13 to about 14, about 13 to about 15, or about 14 to about 15. In still other aspects of this embodiment, the number of doses administered in a multi-dose administration is from, e.g., about 2 to about 5, about 2 to about 10, about 2 to about 15, about 5 to about 10, about 5 to about 15, or about 10 to about 15.

A multi-dose amount of a composition comprising a hyper-concentrated hyaluronidase used to effectively reduce or eliminate a hyaluronic acid-induced blockage in one or more blood vessels of an area of tissue being affected by the blockage can include a total amount of about 250 IU/mL to about 500,000 IU/mL of a hyaluronidase. In aspects of this embodiment, a multi-dose amount of a composition comprising a hyper-concentrated hyaluronidase used to effectively reduce or eliminate a hyaluronic acid-induced blockage in one or more blood vessels of an area of tissue being affected by the blockage can include a total amount of hyaluronidase that is, e.g., at least 25,000 IU/mL, at least 50,000 IU/mL, at least 75,000 IU/mL, at least 100,000 IU/mL, at least 125,000 IU/mL, at least 150,000 IU/mL, at least 175,000 IU/mL, at least 200,000 IU/mL, at least 225,000 IU/mL, at least 250,000 IU/mL, at least 300,000 IU/mL, at least 350,000 IU/mL, at least 400,000 IU/mL, at least 450,000 IU/mL, or at least 500,000 IU/mL. In other aspects of this embodiment, a multi-dose amount of a composition comprising a hyper-concentrated hyaluronidase used to effectively reduce or eliminate a hyaluronic acid-induced blockage in one or more blood vessels of an area of tissue being affected by the blockage can include a total amount of hyaluronidase that is, e.g., at most 25,000 IU/mL, at most 50,000 IU/mL, at most 75,000 IU/mL, at most 100,000 IU/mL, at most 125,000 IU/mL, at most 150,000 IU/mL, at most 175,000 IU/mL, at most 200,000 IU/mL, at most 225,000 IU/mL, at most 250,000 IU/mL, at most 300,000 IU/mL, at most 350,000 IU/mL, at most 400,000 IU/mL, at most 450,000 IU/mL, or at most 500,000 IU/mL.

In yet other aspects of this embodiment, a multi-dose amount of a composition comprising a hyper-concentrated hyaluronidase used to effectively reduce or eliminate a hyaluronic acid-induced blockage in one or more blood vessels of an area of tissue being affected by the blockage can include a total amount of hyaluronidase that is, e.g., about 25,000 IU/mL to about 50,000 IU/mL, about 25,000 IU/mL to about 75,000 IU/mL, about 25,000 IU/mL to about 100,000 IU/mL, about 25,000 IU/mL to about 125,000 IU/mL, about 25,000 IU/mL to about 150,000 IU/mL, about 25,000 IU/mL to about 175,000 IU/mL, about 25,000 IU/mL to about 200,000 IU/mL, about 25,000 IU/mL to about 225,000 IU/mL, about 25,000 IU/mL to about 250,000 IU/mL, about 25,000 IU/mL to about 300,000 IU/mL, about 25,000 IU/mL to about 350,000 IU/mL, about 25,000 IU/mL to about 400,000 IU/mL, about 25,000 IU/mL to about 450,000 IU/mL, about 25,000 IU/mL to about 500,000 IU/mL, about 50,000 IU/mL to about 75,000 IU/mL, about 50,000 IU/mL to about 100,000 IU/mL, about 50,000 IU/mL to about 125,000 IU/mL, about 50,000 IU/mL to about 150,000 IU/mL, about 50,000 IU/mL to about 175,000 IU/mL, about 50,000 IU/mL to about 200,000 IU/mL, about 50,000 IU/mL to about 225,000 IU/mL, about 50,000 IU/mL to about 250,000 IU/mL, about 50,000 IU/mL to about 300,000 IU/mL, about 50,000 IU/mL to about 350,000 IU/mL, about 50,000 IU/mL to about 400,000 IU/mL, about 50,000 IU/mL to about 450,000 IU/mL, about 50,000 IU/mL to about 500,000 IU/mL, about 75,000 IU/mL to about 100,000 IU/mL, about 75,000 IU/mL to about 125,000 IU/mL, about 75,000 IU/mL to about 150,000 IU/mL, about 75,000 IU/mL to about 175,000 IU/mL, about 75,000 IU/mL to about 200,000 IU/mL, about 75,000 IU/mL to about 225,000 IU/mL, about 75,000 IU/mL to about 250,000 IU/mL, about 75,000 IU/mL to about 300,000 IU/mL, about 75,000 IU/mL to about 350,000 IU/mL, about 75,000 IU/mL to about 400,000 IU/mL, about 75,000 IU/mL to about 450,000 IU/mL, about 75,000 IU/mL to about 500,000 IU/mL, about 100,000 IU/mL to about 125,000 IU/mL, about 100,000 IU/mL to about 150,000 IU/mL, about 100,000 IU/mL to about 175,000 IU/mL, about 100,000 IU/mL to about 200,000 IU/mL, about 100,000 IU/mL to about 225,000 IU/mL, about 100,000 IU/mL to about 250,000 IU/mL, about 100,000 IU/mL to about 300,000 IU/mL, about 100,000 IU/mL to about 350,000 IU/mL, about 100,000 IU/mL to about 400,000 IU/mL, about 100,000 IU/mL to about 450,000 IU/mL, about 100,000 IU/mL to about 500,000 IU/mL, about 150,000 IU/mL to about 200,000 IU/mL, about 150,000 IU/mL to about 225,000 IU/mL, about 150,000 IU/mL to about 250,000 IU/mL, about 150,000 IU/mL to about 300,000 IU/mL, about 150,000 IU/mL to about 350,000 IU/mL, about 150,000 IU/mL to about 400,000 IU/mL, about 150,000 IU/mL to about 450,000 IU/mL, about 150,000 IU/mL to about 500,000 IU/mL, about 200,000 IU/mL to about 225,000 IU/mL, about 200,000 IU/mL to about 250,000 IU/mL, about 200,000 IU/mL to about 300,000 IU/mL, about 200,000 IU/mL to about 350,000 IU/mL, about 200,000 IU/mL to about 400,000 IU/mL, about 200,000 IU/mL to about 450,000 IU/mL, about 200,000 IU/mL to about 500,000 IU/mL, about 250,000 IU/mL to about 300,000 IU/mL, about 250,000 IU/mL to about 350,000 IU/mL, about 250,000 IU/mL to about 400,000 IU/mL, about 250,000 IU/mL to about 450,000 IU/mL, about 250,000 IU/mL to about 500,000 IU/mL, about 300,000 IU/mL to about 350,000 IU/mL, about 300,000 IU/mL to about 400,000 IU/mL, about 300,000 IU/mL to about 450,000 IU/mL, about 300,000 IU/mL to about 500,000 IU/mL, about 350,000 IU/mL to about 400,000 IU/mL, about 350,000 IU/mL to about 450,000 IU/mL, about 350,000 IU/mL to about 500,000 IU/mL, about 400,000 IU/mL to about 450,000 IU/mL, about 400,000 IU/mL to about 500,000 IU/mL, or about 450,000 IU/mL to about 500,000 IU/mL.

In aspects of this embodiment, a multi-dose amount of a composition comprising a hyper-concentrated hyaluronidase used to effectively reduce or eliminate a hyaluronic acid-induced blockage in one or more blood vessels of an area of tissue being affected by the blockage can include a total amount of hyaluronidase that is, e.g., at least 10,000 IU/mL, at least 20,000 IU/mL, at least 30,000 IU/mL, at least 40,000 IU/mL, at least 50,000 IU/mL, at least 60,000 IU/mL, at least 70,000 IU/mL, at least 80,000 IU/mL, at least 90,000 IU/mL, or at least 100,000 IU/mL. In other aspects of this embodiment, a multi-dose amount of a composition comprising a hyper-concentrated hyaluronidase used to effectively reduce or eliminate a hyaluronic acid-induced blockage in one or more blood vessels of an area of tissue being affected by the blockage can include a total amount of hyaluronidase that is, e.g., at most 10,000 IU/mL, at most 20,000 IU/mL, at most 30,000 IU/mL, at most 40,000 IU/mL, at most 50,000 IU/mL, at most 60,000 IU/mL, at most 70,000 IU/mL, at most 80,000 IU/mL, at most 90,000 IU/mL, or at most 100,000 IU/mL.

In yet other aspects of this embodiment, a multi-dose amount of a composition comprising a hyper-concentrated hyaluronidase used to effectively reduce or eliminate a hyaluronic acid-induced blockage in one or more blood vessels of an area of tissue being affected by the blockage can include a total amount of hyaluronidase that is, e.g., about 10,000 IU/mL to about 20,000 IU/mL, about 10,000 IU/mL to about 30,000 IU/mL, about 10,000 IU/mL to about 40,000 IU/mL, about 10,000 IU/mL to about 50,000 IU/mL, about 10,000 IU/mL to about 60,000 IU/mL, about 10,000 IU/mL to about 70,000 IU/mL, about 10,000 IU/mL to about 80,000 IU/mL, about 10,000 IU/mL to about 90,000 IU/mL, about 10,000 IU/mL to about 100,000 IU/mL, about 20,000 IU/mL to about 30,000 IU/mL, about 20,000 IU/mL to about 40,000 IU/mL, about 20,000 IU/mL to about 50,000 IU/mL, about 20,000 IU/mL to about 60,000 IU/mL, about 20,000 IU/mL to about 70,000 IU/mL, about 20,000 IU/mL to about 80,000 IU/mL, about 20,000 IU/mL to about 90,000 IU/mL, about 20,000 IU/mL to about 100,000 IU/mL, about 30,000 IU/mL to about 40,000 IU/mL, about 30,000 IU/mL to about 50,000 IU/mL, about 30,000 IU/mL to about 60,000 IU/mL, about 30,000 IU/mL to about 70,000 IU/mL, about 30,000 IU/mL to about 80,000 IU/mL, about 30,000 IU/mL to about 90,000 IU/mL, about 30,000 IU/mL to about 100,000 IU/mL, about 40,000 IU/mL to about 50,000 IU/mL, about 40,000 IU/mL to about 60,000 IU/mL, about 40,000 IU/mL to about 70,000 IU/mL, about 40,000 IU/mL to about 80,000 IU/mL, about 40,000 IU/mL to about 90,000 IU/mL, about 40,000 IU/mL to about 100,000 IU/mL, about 50,000 IU/mL to about 60,000 IU/mL, about 50,000 IU/mL to about 70,000 IU/mL, about 50,000 IU/mL to about 80,000 IU/mL, about 50,000 IU/mL to about 90,000 IU/mL, about 50,000 IU/mL to about 100,000 IU/mL, about 60,000 IU/mL to about 70,000 IU/mL, about 60,000 IU/mL to about 80,000 IU/mL, about 60,000 IU/mL to about 90,000 IU/mL, about 60,000 IU/mL to about 100,000 IU/mL, about 70,000 IU/mL to about 80,000 IU/mL, about 70,000 IU/mL to about 90,000 IU/mL, about 70,000 IU/mL to about 100,000 IU/mL, about 80,000 IU/mL to about 90,000 IU/mL, about 80,000 IU/mL to about 100,000 IU/mL, or about 90,000 IU/mL to about 100,000 IU/mL.

In aspects of this embodiment, a multi-dose amount of a composition comprising a hyper-concentrated hyaluronidase used to effectively reduce or eliminate a hyaluronic acid-induced blockage in one or more blood vessels of an area of tissue being affected by the blockage can include a total amount of hyaluronidase that is, e.g., at least 5,000 IU/mL, at least 6,000 IU/mL, at least 7,000 IU/mL, at least 8,000 IU/mL, at least 9,000 IU/mL, at least 10,000 IU/mL, at least 11,000 IU/mL, at least 12,000 IU/mL, at least 13,000 IU/mL, at least 14,000 IU/mL, at least 15,000 IU/mL, at least 16,000 IU/mL, at least 17,000 IU/mL, at least 18,000 IU/mL, at least 19,000 IU/mL, or at least 20,000 IU/mL. In other aspects of this embodiment, a multi-dose amount of a composition comprising a hyper-concentrated hyaluronidase used to effectively reduce or eliminate a hyaluronic acid-induced blockage in one or more blood vessels of an area of tissue being affected by the blockage can include a total amount of hyaluronidase that is, e.g., at most 5,000 IU/mL, at most 6,000 IU/mL, at most 7,000 IU/mL, at most 8,000 IU/mL, at most 9,000 IU/mL, at most 10,000 IU/mL, at most 11,000 IU/mL, at most 12,000 IU/mL, at most 13,000 IU/mL, at most 14,000 IU/mL, at most 15,000 IU/mL, at most 16,000 IU/mL, at most 17,000 IU/mL, at most 18,000 IU/mL, at most 19,000 IU/mL, or at most 20,000 IU/mL.

In yet other aspects of this embodiment, a multi-dose amount of a composition comprising a hyper-concentrated hyaluronidase used to effectively reduce or eliminate a hyaluronic acid-induced blockage in one or more blood vessels of an area of tissue being affected by the blockage can include a total amount of hyaluronidase that is, e.g., about 5,000 IU/mL to about 6,000 IU/mL, about 5,000 IU/mL to about 7,000 IU/mL, about 5,000 IU/mL to about 8,000 IU/mL, about 5,000 IU/mL to about 9,000 IU/mL, about 5,000 IU/mL to about 10,000 IU/mL, about 5,000 IU/mL to about 11,000 IU/mL, about 5,000 IU/mL to about 12,000 IU/mL, about 5,000 IU/mL to about 13,000 IU/mL, about 5,000 IU/mL to about 14,000 IU/mL, about 5,000 IU/mL to about 15,000 IU/mL, about 5,000 IU/mL to about 16,000 IU/mL, about 5,000 IU/mL to about 17,000 IU/mL, about 5,000 IU/mL to about 18,000 IU/mL, about 5,000 IU/mL to about 19,000 IU/mL, about 5,000 IU/mL to about 20,000 IU/mL, about 6,000 IU/mL to about 7,000 IU/mL, about 6,000 IU/mL to about 8,000 IU/mL, about 6,000 IU/mL to about 9,000 IU/mL, about 6,000 IU/mL to about 10,000 IU/mL, about 6,000 IU/mL to about 11,000 IU/mL, about 6,000 IU/mL to about 12,000 IU/mL, about 6,000 IU/mL to about 13,000 IU/mL, about 6,000 IU/mL to about 14,000 IU/mL, about 6,000 IU/mL to about 15,000 IU/mL, about 6,000 IU/mL to about 16,000 IU/mL, about 6,000 IU/mL to about 17,000 IU/mL, about 6,000 IU/mL to about 18,000 IU/mL, about 6,000 IU/mL to about 19,000 IU/mL, about 6,000 IU/mL to about 20,000 IU/mL, about 7,000 IU/mL to about 8,000 IU/mL, about 7,000 IU/mL to about 9,000 IU/mL, about 7,000 IU/mL to about 10,000 IU/mL, about 7,000 IU/mL to about 11,000 IU/mL, about 7,000 IU/mL to about 12,000 IU/mL, about 7,000 IU/mL to about 13,000 IU/mL, about 7,000 IU/mL to about 14,000 IU/mL, about 7,000 IU/mL to about 15,000 IU/mL, about 7,000 IU/mL to about 16,000 IU/mL, about 7,000 IU/mL to about 17,000 IU/mL, about 7,000 IU/mL to about 18,000 IU/mL, about 7,000 IU/mL to about 19,000 IU/mL, about 7,000 IU/mL to about 20,000 IU/mL, about 8,000 IU/mL to about 9,000 IU/mL, about 8,000 IU/mL to about 10,000 IU/mL, about 8,000 IU/mL to about 11,000 IU/mL, about 8,000 IU/mL to about 12,000 IU/mL, about 8,000 IU/mL to about 13,000 IU/mL, about 8,000 IU/mL to about 14,000 IU/mL, about 8,000 IU/mL to about 15,000 IU/mL, about 8,000 IU/mL to about 16,000 IU/mL, about 8,000 IU/mL to about 17,000 IU/mL, about 8,000 IU/mL to about 18,000 IU/mL, about 8,000 IU/mL to about 19,000 IU/mL, about 8,000 IU/mL to about 20,000 IU/mL, about 9,000 IU/mL to about 10,000 IU/mL, about 9,000 IU/mL to about 11,000 IU/mL, about 9,000 IU/mL to about 12,000 IU/mL, about 9,000 IU/mL to about 13,000 IU/mL, about 9,000 IU/mL to about 14,000 IU/mL, about 9,000 IU/mL to about 15,000 IU/mL, about 9,000 IU/mL to about 16,000 IU/mL, about 9,000 IU/mL to about 17,000 IU/mL, about 9,000 IU/mL to about 18,000 IU/mL, about 9,000 IU/mL to about 19,000 IU/mL, about 9,000 IU/mL to about 20,000 IU/mL, about 10,000 IU/mL to about 11,000 IU/mL, about 10,000 IU/mL to about 12,000 IU/mL, about 10,000 IU/mL to about 13,000 IU/mL, about 10,000 IU/mL to about 14,000 IU/mL, about 10,000 IU/mL to about 15,000 IU/mL, about 10,000 IU/mL to about 16,000 IU/mL, about 10,000 IU/mL to about 17,000 IU/mL, about 10,000 IU/mL to about 18,000 IU/mL, about 10,000 IU/mL to about 19,000 IU/mL, about 10,000 IU/mL to about 20,000 IU/mL, about 11,000 IU/mL to about 12,000 IU/mL, about 11,000 IU/mL to about 13,000 IU/mL, about 11,000 IU/mL to about 14,000 IU/mL, about 11,000 IU/mL to about 15,000 IU/mL, about 11,000 IU/mL to about 16,000 IU/mL, about 11,000 IU/mL to about 17,000 IU/mL, about 11,000 IU/mL to about 18,000 IU/mL, about 11,000 IU/mL to about 19,000 IU/mL, about 11,000 IU/mL to about 20,000 IU/mL, about 12,000 IU/mL to about 13,000 IU/mL, about 12,000 IU/mL to about 14,000 IU/mL, about 12,000 IU/mL to about 15,000 IU/mL, about 12,000 IU/mL to about 16,000 IU/mL, about 12,000 IU/mL to about 17,000 IU/mL, about 12,000 IU/mL to about 18,000 IU/mL, about 12,000 IU/mL to about 19,000 IU/mL, about 12,000 IU/mL to about 20,000 IU/mL, about 13,000 IU/mL to about 14,000 IU/mL, about 13,000 IU/mL to about 15,000 IU/mL, about 13,000 IU/mL to about 16,000 IU/mL, about 13,000 IU/mL to about 17,000 IU/mL, about 13,000 IU/mL to about 18,000 IU/mL, about 13,000 IU/mL to about 19,000 IU/mL, about 13,000 IU/mL to about 20,000 IU/mL, about 14,000 IU/mL to about 15,000 IU/mL, about 14,000 IU/mL to about 16,000 IU/mL, about 14,000 IU/mL to about 17,000 IU/mL, about 14,000 IU/mL to about 18,000 IU/mL, about 14,000 IU/mL to about 19,000 IU/mL, about 14,000 IU/mL to about 20,000 IU/mL, about 15,000 IU/mL to about 16,000 IU/mL, about 15,000 IU/mL to about 17,000 IU/mL, about 15,000 IU/mL to about 18,000 IU/mL, about 15,000 IU/mL to about 19,000 IU/mL, about 15,000 IU/mL to about 20,000 IU/mL, about 16,000 IU/mL to about 17,000 IU/mL, about 16,000 IU/mL to about 18,000 IU/mL, about 16,000 IU/mL to about 19,000 IU/mL, about 16,000 IU/mL to about 20,000 IU/mL, about 17,000 IU/mL to about 18,000 IU/mL, about 17,000 IU/mL to about 19,000 IU/mL, about 17,000 IU/mL to about 20,000 IU/mL, about 18,000 IU/mL to about 19,000 IU/mL, about 18,000 IU/mL to about 20,000 IU/mL, or about 19,000 IU/mL to about 20,000 IU/mL.

In aspects of this embodiment, a multi-dose amount of a composition comprising a hyper-concentrated hyaluronidase used to effectively reduce or eliminate a hyaluronic acid-induced blockage in one or more blood vessels of an area of tissue being affected by the blockage can include a total amount of hyaluronidase that is, e.g., at least 250 IU/mL, at least 500 IU/mL, at least 750 IU/mL, at least 1,000 IU/mL, at least 1,500 IU/mL, at least 2,000 IU/mL, at least 2,500 IU/mL, at least 3,000 IU/mL, at least 3,500 IU/mL, at least 4,000 IU/mL, at least 4,500 IU/mL, or at least 5,000 IU/mL. In other aspects of this embodiment, a multi-dose amount of a composition comprising a hyper-concentrated hyaluronidase used to effectively reduce or eliminate a hyaluronic acid-induced blockage in one or more blood vessels of an area of tissue being affected by the blockage can include a total amount of hyaluronidase that is, e.g., at most 250 IU/mL, at most 500 IU/mL, at most 750 IU/mL, at most 1,000 IU/mL, at most 1,500 IU/mL, at most 2,000 IU/mL, at most 2,500 IU/mL, at most 3,000 IU/mL, at most 3,500 IU/mL, at most 4,000 IU/mL, at most 4,500 IU/mL, or at most 5,000 IU/mL.

In yet other aspects of this embodiment, a multi-dose amount of a composition comprising a hyper-concentrated hyaluronidase used to effectively reduce or eliminate a hyaluronic acid-induced blockage in one or more blood vessels of an area of tissue being affected by the blockage can include a total amount of hyaluronidase that is, e.g., about 250 IU/mL to about 500 IU/mL, about 250 IU/mL to about 750 IU/mL, about 250 IU/mL to about 1,000 IU/mL, about 250 IU/mL to about 1,500 IU/mL, about 250 IU/mL to about 2,000 IU/mL, about 250 IU/mL to about 2,500 IU/mL, about 250 IU/mL to about 3,000 IU/mL, about 250 IU/mL to about 3,500 IU/mL, about 250 IU/mL to about 4,000 IU/mL, about 250 IU/mL to about 4,500 IU/mL, about 250 IU/mL to about 5,000 IU/mL, about 500 IU/mL to about 750 IU/mL, about 500 IU/mL to about 1,000 IU/mL, about 500 IU/mL to about 1,500 IU/mL, about 500 IU/mL to about 2,000 IU/mL, about 500 IU/mL to about 2,500 IU/mL, about 500 IU/mL to about 3,000 IU/mL, about 500 IU/mL to about 3,500 IU/mL, about 500 IU/mL to about 4,000 IU/mL, about 500 IU/mL to about 4,500 IU/mL, about 500 IU/mL to about 5,000 IU/mL, about 750 IU/mL to about 1,000 IU/mL, about 750 IU/mL to about 1,500 IU/mL, about 750 IU/mL to about 2,000 IU/mL, about 750 IU/mL to about 2,500 IU/mL, about 750 IU/mL to about 3,000 IU/mL, about 750 IU/mL to about 3,500 IU/mL, about 750 IU/mL to about 4,000 IU/mL, about 750 IU/mL to about 4,500 IU/mL, about 750 IU/mL to about 5,000 IU/mL, about 1,000 IU/mL to about 1,500 IU/mL, about 1,000 IU/mL to about 2,000 IU/mL, about 1,000 IU/mL to about 2,500 IU/mL, about 1,000 IU/mL to about 3,000 IU/mL, about 1,000 IU/mL to about 3,500 IU/mL, about 1,000 IU/mL to about 4,000 IU/mL, about 1,000 IU/mL to about 4,500 IU/mL, about 1,000 IU/mL to about 5,000 IU/mL, about 1,500 IU/mL to about 2,000 IU/mL, about 1,500 IU/mL to about 2,500 IU/mL, about 1,500 IU/mL to about 3,000 IU/mL, about 1,500 IU/mL to about 3,500 IU/mL, about 1,500 IU/mL to about 4,000 IU/mL, about 1,500 IU/mL to about 4,500 IU/mL, about 1,500 IU/mL to about 5,000 IU/mL, about 2,000 IU/mL to about 2,500 IU/mL, about 2,000 IU/mL to about 3,000 IU/mL, about 2,000 IU/mL to about 3,500 IU/mL, about 2,000 IU/mL to about 4,000 IU/mL, about 2,000 IU/mL to about 4,500 IU/mL, about 2,000 IU/mL to about 5,000 IU/mL, about 2,500 IU/mL to about 3,000 IU/mL, about 2,500 IU/mL to about 3,500 IU/mL, about 2,500 IU/mL to about 4,000 IU/mL, about 2,500 IU/mL to about 4,500 IU/mL, about 2,500 IU/mL to about 5,000 IU/mL, about 3,000 IU/mL to about 3,500 IU/mL, about 3,000 IU/mL to about 4,000 IU/mL, about 3,000 IU/mL to about 4,500 IU/mL, about 3,000 IU/mL to about 5,000 IU/mL, about 3,500 IU/mL to about 4,000 IU/mL, about 3,500 IU/mL to about 4,500 IU/mL, about 3,500 IU/mL to about 5,000 IU/mL, about 4,000 IU/mL to about 4,500 IU/mL, about 4,000 IU/mL to about 5,000 IU/mL, or about 4,500 IU/mL to about 5,000 IU/mL.

The tissue that is affected by a particular hyaluronic acid-induced blockage in one or more blood vessels will influence how quickly administration of a hyper-concentrated hyaluronidase will need to occur in order to avoid irreparable damage to the tissue. For example, one type of tissue can be designated as hypersensitive tissue. Hypersensitive tissue is typically well-differentiated tissue that is very sensitive to oxygen deprivation and will undergo irreversible damage if blood flow is not restored within about 5 to 15 minutes. For example, brain tissue is highly susceptible to damage if it is not re-perfused with blood within about 5 minutes. Similarly, retinal tissue is highly sensitive to discontinued blood flow without suffering irreparable damage, with studies suggesting irreversible damage can occur within about 5 to 15 minutes.

In contrast, hyposensitive tissue is generally classified as not well-differentiated and can avoid irreparable damage during discontinued blood flow for much longer than can hypersensitive tissue. Tissues such as skin, muscle, fat, and bone can avoid irreparable damage during discontinued blood flow for prolonged periods of time (many hours to several days, but no one knows for certain) before suffering irreversible damage.

Ideally, it is advantageous to restore blood flow to all types of tissue as quickly as possible when discontinuation of blood flow occurs. Although hyposensitive tissue may be able to avoid irreparable damage for a longer period of time than hypersensitive tissue during discontinued blood flow, it is best to re-perfuse all types of tissues as quickly as possible in order to minimize the risk of irreparable damage.

A composition comprising a hyper-concentrated hyaluronidase disclosed herein is formulated to rapidly cover an area of tissue being affected by the blockage. Such rapid expansion ensures that sufficient amounts of a hyaluronidase are transported to one or more blood vessels supplying the affected tissue within about 90 minutes or less. In aspects of this embodiment, a composition comprising a hyper-concentrated hyaluronidase disclosed herein is formulated to rapidly cover an area of tissue being affected by the blockage in, e.g., at most 15 minutes, at most 30 minutes, at most 45 minutes, at most 60 minutes, at most 75 minutes or at most 90 minutes. In other aspects of this embodiment, a composition comprising a hyper-concentrated hyaluronidase disclosed herein is formulated to rapidly cover an area of tissue being affected by the blockage in, e.g., about 5 minutes to about 10 minutes, about 5 minutes to about 15 minutes, about 5 minutes to about 30 minutes, about 5 to about 45 minutes, about 5 to about 60 minutes, about 5 to about 75 minutes, about 5 to about 90 minutes, about 10 minutes to about 15 minutes, about 10 minutes to about 30 minutes, about 10 to about 45 minutes, about 10 to about 60 minutes, about 10 to about 75 minutes, about 10 to about 90 minutes, about 15 minutes to about 30 minutes, about 15 to about 45 minutes, about 15 to about 60 minutes, about 15 to about 75 minutes, about 15 to about 90 minutes, about 15 minutes to about 45 minutes, about 15 minutes to about 60 minutes, about 15 minutes to about 75 minutes, or about 15 minutes to about 90 minutes, about 30 to about 45 minutes, about 30 to about 60 minutes, about 30 to about 75 minutes, about 30 to about 90 minutes, about 45 to about 60 minutes, about 45 to about 75 minutes, about 45 to about 90 minutes, about 60 to about 75 minutes, about 60 to about 90 minutes, or about 75 to about 90 minutes.

In some embodiments, the rapid expansion ensures that sufficient amounts of a hyaluronidase are transported to one or more blood vessels supplying the affected area of tissue within about 5 to about 15 minutes or less. In aspects of this embodiment, a composition comprising a hyper-concentrated hyaluronidase disclosed herein is formulated to rapidly cover an area of tissue being affected by the blockage in, e.g., at most 1 minute, at most 2 minutes, at most 3 minutes, at most 4 minutes, at most 5 minutes, at most 10 minutes, or at most 15 minutes. In other aspects of this embodiment, a composition comprising a hyper-concentrated hyaluronidase disclosed herein is formulated to rapidly cover an area of tissue being affected by the blockage in, e.g., about 1 minute to about 3 minutes, about 1 minute to about 5 minutes, about 1 minute to about 7 minutes, about 1 minute to about 10 minutes, about 1 minute to about 15 minutes, about 3 minutes to about 5 minutes, about 3 minutes to about 7 minutes, about 3 minutes to about 10 minutes, about 3 minutes to about 15 minutes, about 5 minutes to about 7 minutes, about 5 minutes to about 10 minutes, about 5 minutes to about 15 minutes, or about 10 minutes to about 15 minutes.

The volume of a composition comprising a hyper-concentrated hyaluronidase delivered to an area of tissue being affected by the blockage can range from about 0.5 mL to about 5.0 mL. In aspects of this embodiment, a volume of a composition comprising a hyper-concentrated hyaluronidase delivered to an area of tissue being affected by the blockage can be, e.g., at least 0.5 mL, at least 1.0 mL, at least 2.0 mL, at least 3.0 mL, at least 4.0 mL, or at least 5.0 mL. In other aspects of this embodiment, a volume of a composition comprising a hyper-concentrated hyaluronidase delivered to an area of tissue being affected by the blockage can be, e.g., at most 0.5 mL, at most 1.0 mL, at most 2.0 mL, at most 3.0 mL, at most 4.0 mL, or at most 5.0 mL. In yet other aspects of this embodiment, a volume of a composition comprising a hyper-concentrated hyaluronidase delivered to an area of tissue being affected by the blockage can be, e.g., about 0.5 mL to 1.0 mL, about 0.5 mL to 2.0 mL, about 0.5 mL to 3.0 mL, about 0.5 mL to 4.0 mL, about 0.5 mL to 5.0 mL, about 1.0 mL to 2.0 mL, about 1.0 mL to 3.0 mL, about 1.0 mL to 4.0 mL, about 1.0 to 5.0 mL, about 2.0 mL to 3.0 mL, about 2.0 mL to 4.0 mL, about 2.0 to 5.0 mL, about 3.0 mL to 4.0 mL, about 3.0 to 5.0 mL, or about 4.0 to 5.0 mL.

Aspects of the present specification disclose a delivery system. Non-limiting examples of a delivery system include, e.g., a manual injecting device like a needle and syringe, or an ADG needle, as well as an auto-injecting device like an autoinjector. Non-limiting examples of needle gauge useful for a delivery system, such as, e.g., a manual or auto-injecting device, include 21 gauge, 22 gauge, 23 gauge, 24 gauge, 25 gauge, 26 gauge, 27 gauge, 28 gauge, 29 gauge, 30 gauge, 31 gauge, 32 gauge, and 33 gauge.

Aspects of the present specification disclose methods of reducing or eliminating a hyaluronic acid-induced blockage in one or more blood vessels supplying blood to an area of tissue. The disclosed methods comprise administering a composition comprising a hyper-concentrated hyaluronidase to an individual in a manner that reduces or eliminates a hyaluronic acid-induced blockage in one or more blood vessels supplying blood to an area of tissue being affected by the blockage. In aspects, the disclosed methods administer composition comprising a hyper-concentrated hyaluronidase to a dermal portion of a skin region. In other aspects, the disclosed methods administer a composition comprising a hyper-concentrated hyaluronidase using a delivery system disclosed herein. The disclosed methods can administer a composition comprising a hyper-concentrated hyaluronidase in a single- or multi-dose amount ranging from about 10,000 IU to about 20,000 IU of a hyaluronidase.

In aspects of this embodiment, reducing or eliminating a hyaluronic acid-induced blockage in one or more blood vessels supplying blood to an area of tissue reduces or inhibits one or more physiological conditions or symptoms associated with a hyaluronic acid-induced blockage by, e.g., at least 10%, at least 20%, at least 30%, at least 40%, at least 50%, at least 60%, at least 70%, at least 80%, at least 90% or at least 95% or more. In other aspects of this embodiment, reducing or eliminating a hyaluronic acid-induced blockage in one or more blood vessels supplying blood to an area of tissue reduces or inhibits one or more physiological conditions or symptoms associated with a hyaluronic acid-induced blockage by, e.g., at most 10%, at most 20%, at most 30%, at most 40%, at most 50%, at most 60%, at most 70%, at most 80%, at most 90% or at most 100%. In yet other aspects of this embodiment, reducing or eliminating a hyaluronic acid-induced blockage in one or more blood vessels supplying blood to an area of tissue reduces or inhibits one or more physiological conditions or symptoms associated with a hyaluronic acid-induced blockage by, e.g., about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 20% to about 100%, about 20% to about 90%, about 20% to about 80%, about 20% to about 70%, about 20% to about 60%, about 20% to about 50%, about 20% to about 40%, about 20% to about 30%, about 30% to about 100%, about 30% to about 90%, about 30% to about 80%, about 30% to about 70%, about 30% to about 60%, about 30% to about 50%, about 30% to about 40%, about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%. In still other aspects of this embodiment, reducing or eliminating a hyaluronic acid-induced blockage in one or more blood vessels supplying blood to an area of tissue reduces or inhibits one or more physiological conditions or symptoms associated with a hyaluronic acid-induced blockage for, e.g., at least one week, at least one month, at least two months, at least three months, at least four months, at least five months, at least six months, at least seven months, at least eight months, at least nine months, at least ten months, at least eleven months, or at least twelve months.

Aspects of the present specification disclose methods of reducing or inhibiting a vascular occlusion in an area of tissue of an individual in need thereof. A vascular occlusion is reduced or inhibited by reducing or eliminating a hyaluronic acid-induced blockage in one or more blood vessels supplying blood to an area of tissue being affected by the blockage. The disclosed methods comprise administering a composition comprising a hyper-concentrated hyaluronidase to an individual in a manner that reduces or eliminates a hyaluronic acid-induced blockage in one or more blood vessels supplying blood to an area of tissue being affected by the blockage, thereby reducing or inhibiting the vascular occlusion in the area of tissue. In aspects, the disclosed methods administer composition comprising a hyper-concentrated hyaluronidase to a dermal portion of a skin region. In other aspects, the disclosed methods administer a composition comprising a hyper-concentrated hyaluronidase using a delivery system disclosed herein. The disclosed methods can administer a composition comprising a hyper-concentrated hyaluronidase in a single- or multi-dose amount ranging from about 10,000 IU to about 20,000 IU of a hyaluronidase.

In aspects of this embodiment, reducing or inhibiting a vascular occlusion in an area of tissue reduces or inhibits one or more physiological conditions or symptoms associated with a hyaluronic acid-induced blockage in one or more blood vessels by, e.g., at least 10%, at least 20%, at least 30%, at least 40%, at least 50%, at least 60%, at least 70%, at least 80%, at least 90% or at least 100%. In other aspects of this embodiment, reducing or inhibiting a vascular occlusion in an area of tissue reduces or inhibits one or more physiological conditions or symptoms associated with a hyaluronic acid-induced blockage in one or more blood vessels by, e.g., at most 10%, at most 20%, at most 30%, at most 40%, at most 50%, at most 60%, at most 70%, at most 80%, at most 90% or at most 100%. In yet other aspects of this embodiment, reducing or inhibiting a vascular occlusion in an area of tissue reduces or inhibits one or more physiological conditions or symptoms associated with a hyaluronic acid-induced blockage in one or more blood vessels by, e.g., about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 20% to about 100%, about 20% to about 90%, about 20% to about 80%, about 20% to about 70%, about 20% to about 60%, about 20% to about 50%, about 20% to about 40%, about 20% to about 30%, about 30% to about 100%, about 30% to about 90%, about 30% to about 80%, about 30% to about 70%, about 30% to about 60%, about 30% to about 50%, about 30% to about 40%, about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%. In still other aspects of this embodiment, reducing or inhibiting a vascular occlusion in an area of tissue reduces or inhibits one or more physiological conditions or symptoms associated with a hyaluronic acid-induced blockage in one or more blood vessels for, e.g., at least one week, at least one month, at least two months, at least three months, at least four months, at least five months, at least six months, at least seven months, at least eight months, at least nine months, at least ten months, at least eleven months, or at least twelve months.

Aspects of the present specification disclose methods of reducing or inhibiting hyaluronic acid-induced loss of tissue in an individual in need thereof. A hyaluronic acid-induced loss of tissue is reduced or inhibited by reducing or eliminating a hyaluronic acid-induced blockage in one or more blood vessels supplying blood to an area of tissue being affected by the blockage. The disclosed methods comprise administering a composition comprising a hyper-concentrated hyaluronidase to an individual in a manner that reduces or eliminates a hyaluronic acid-induced blockage in one or more blood vessels supplying blood to an area of tissue being affected by the blockage, thereby reducing or inhibiting the hyaluronic acid-induced loss of tissue in the individual. In aspects, the disclosed methods administer a composition comprising a hyaluronidase to a dermal portion of a skin region. In other aspects, the disclosed methods administer a composition comprising a hyper-concentrated hyaluronidase using a delivery system disclosed herein. The disclosed methods can administer a composition comprising a hyper-concentrated hyaluronidase in a single- or multi-dose amount ranging from about 10,000 IU to about 20,000 IU of a hyaluronidase.

In some embodiments, when the methods disclosed herein comprise multiple doses of a hyper-concentrated hyaluronidase, such administrations can occur in rapid succession. In aspects of these embodiments, 2 to 10 doses of a composition comprising a hyper-concentrated hyaluronidase can be administered to an area of tissue being affected by the blockage within 5 to 15 minutes. In other aspects of these embodiments, 2 to 7 doses of a composition comprising a hyper-concentrated hyaluronidase can be administered to an area of tissue being affected by the blockage within 5 to 15 minutes. In yet other aspects of these embodiments, 2 to 5 doses of a composition comprising a hyper-concentrated hyaluronidase can be administered to an area of tissue being affected by the blockage within 5 to 15 minutes. In still other aspects of these embodiments, 2 to 3 doses of a composition comprising a hyper-concentrated hyaluronidase can be administered to an area of tissue being affected by the blockage within 5 to 15 minutes. In still other aspects of these embodiments, 2 doses of a composition comprising a hyper-concentrated hyaluronidase can be administered to an area of tissue being affected by the blockage within 5 to 15 minutes.

In aspects of this embodiment, reducing or eliminating a hyaluronic acid-induced blockage in one or more blood vessels supplying blood to an area of tissue restores or maintains one or more qualitative or quantitative aspects of tissue health by, e.g., at least 10%, at least 20%, at least 30%, at least 40%, at least 50%, at least 60%, at least 70%, at least 80%, at least 90% or at least 100%. In other aspects of this embodiment, reducing or eliminating a hyaluronic acid-induced blockage in one or more blood vessels supplying blood to an area of tissue restores or maintains one or more qualitative or quantitative aspects of tissue health by, e.g., at most 10%, at most 20%, at most 30%, at most 40%, at most 50%, at most 60%, at most 70%, at most 80%, at most 90% or at most 100%. In yet other aspects of this embodiment, reducing or eliminating a hyaluronic acid-induced blockage in one or more blood vessels supplying blood to an area of tissue restores or maintains one or more qualitative or quantitative aspects of tissue health by, e.g., about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 20% to about 100%, about 20% to about 90%, about 20% to about 80%, about 20% to about 70%, about 20% to about 60%, about 20% to about 50%, about 20% to about 40%, about 20% to about 30%, about 30% to about 100%, about 30% to about 90%, about 30% to about 80%, about 30% to about 70%, about 30% to about 60%, about 30% to about 50%, about 30% to about 40%, about 40% to about 100%, about 40% to about 90%, about 40% to about 80%, about 40% to about 70%, about 40% to about 60%, about 40% to about 50%, about 50% to about 100%, about 50% to about 90%, about 50% to about 80%, about 50% to about 70%, about 50% to about 60%, about 60% to about 100%, about 60% to about 90%, about 60% to about 80%, about 60% to about 70%, about 70% to about 100%, about 70% to about 90%, about 70% to about 80%, about 80% to about 100%, about 80% to about 90%, or about 90% to about 100%. In still other aspects of this embodiment, reducing or eliminating a hyaluronic acid-induced blockage in one or more blood vessels supplying blood to an area of tissue restores or maintains one or more qualitative or quantitative aspects of tissue health for, e.g., at least one week, at least one month, at least two months, at least three months, at least four months, at least five months, at least six months, at least seven months, at least eight months, at least nine months, at least ten months, at least eleven months, or at least twelve months.

The methods disclosed herein reduce or eliminate a hyaluronic acid-induced blockage in one or more blood vessels supplying blood to an area of tissue. In some embodiments, the methods disclosed herein reduce or eliminate a hyaluronic acid-induced blockage in one or more blood vessels supplying blood to an area of tissue being affected by the blockage within a timeframe that prevents, reduces or stops necrosis, nerve damage, or other organ or tissue damage.

In some embodiments, the methods disclosed herein reduce or eliminate a hyaluronic acid-induced blockage in one or more blood vessels supplying blood to an area of tissue within about 90 minutes or less. In aspects of this embodiment, a hyaluronic acid-induced blockage in one or more blood vessels supplying blood to an area of tissue is reduced or eliminated in, e.g., at most 15 minutes, at most 30 minutes, at most 45 minutes, at most 60 minutes, at most 75 minutes or at most 90 minutes. In other aspects of this embodiment, a hyaluronic acid-induced blockage in one or more blood vessels supplying blood to an area of tissue is reduced or eliminated in, e.g., about 5 minutes to about 10 minutes, about 5 minutes to about 15 minutes, about 5 minutes to about 30 minutes, about 5 to about 45 minutes, about 5 to about 60 minutes, about 5 to about 75 minutes, about 5 to about 90 minutes, about 10 minutes to about 15 minutes, about 10 minutes to about 30 minutes, about 10 to about 45 minutes, about 10 to about 60 minutes, about 10 to about 75 minutes, about 10 to about 90 minutes, about 15 minutes to about 30 minutes, about 15 minutes to about 45 minutes, about 15 minutes to about 60 minutes, about 15 minutes to about 75 minutes, about 15 minutes to about 90 minutes, about 30 to about 45 minutes, about 30 to about 60 minutes, about 30 to about 75 minutes, about 30 to about 90 minutes, about 45 to about 60 minutes, about 45 to about 75 minutes, about 45 to about 90 minutes, about 60 to about 75 minutes, about 60 to about 90 minutes, or about 75 to about 90 minutes.

In some embodiments, the methods disclosed herein reduce or eliminate a hyaluronic acid-induced blockage in one or more blood vessels supplying blood to an area of tissue within about 5 to about 15 minutes or less. In aspects of this embodiment, a hyaluronic acid-induced blockage in one or more blood vessels supplying blood to an area of tissue is reduced or eliminated in, e.g., at most 1 minute, at most 2 minutes, at most 3 minutes, at most 4 minutes, at most 5 minutes, at most 10 minutes, or at most 15 minutes. In other aspects of this embodiment, a hyaluronic acid-induced blockage in one or more blood vessels supplying blood to an area of tissue is reduced or eliminated in, e.g., about 1 minute to about 3 minutes, about 1 minute to about 5 minutes, about 1 minute to about 7 minutes, about 1 minute to about 10 minutes, about 1 minute to about 15 minutes, about 3 minutes to about 5 minutes, about 3 minutes to about 7 minutes, about 3 minutes to about 10 minutes, about 3 minutes to about 15 minutes, about 5 minutes to about 7 minutes, about 5 minutes to about 10 minutes, about 5 minutes to about 15 minutes, about 7 minutes to about 10 minutes, about 7 minutes to about 15 minutes, or about 10 minutes to about 15 minutes.

Aspects of the present specification disclose a kit. In one embodiment, the kit can comprise a container that includes a composition comprising a hyper-concentrated hyaluronidase disclosed herein. In another embodiment, a kit can comprise a plurality of containers, with each such container including a composition comprising a hyper-concentrated hyaluronidase disclosed herein. For example, a kit can comprise 1, 2, 3, 4, 5, 6, 7, 8, 9, 10, 11, 12, 13, 14, 15, 16, 17, 18, 19 or 20 or more containers, with each such container including a composition comprising a hyper-concentrated hyaluronidase disclosed herein. Each of the disclosed containers can comprise a single dose of a composition comprising a hyper-concentrated hyaluronidase disclosed herein, multiple doses of a composition comprising a hyper-concentrated hyaluronidase disclosed herein, or a combination thereof. In addition, each of the disclosed containers can contain a composition comprising a hyper-concentrated hyaluronidase disclosed herein in liquid form or in dried form.

In some embodiments, a container disclosed herein can be a vial or similar vessel containing a composition comprising a hyper-concentrated hyaluronidase disclosed herein, where such composition comprising a hyper-concentrated hyaluronidase disclosed herein would need to be transferred to a delivery system, such as, e.g., a manual or auto-injecting device. In some embodiments, a kit can comprise one or more vials, e.g., 1 to 20 vials, 7 to 15 vials, 8 to 12 vials, or 10 vials, with each such vial containing a composition comprising a hyper-concentrated hyaluronidase disclosed herein and optionally one or more delivery systems, e.g., 1 to 20 delivery systems, 7 to 15 delivery systems, 8 to 12 delivery systems, or 10 delivery systems, with each such delivery system employed to deliver the disclosed compositions when needed.

In some embodiments, a container disclosed herein can be a delivery system containing a composition comprising a hyper-concentrated hyaluronidase disclosed herein. In some embodiments, a kit can comprise one or more delivery systems, e.g., 1 to 20 delivery systems, 7 to 15 delivery systems, 8 to 12 delivery systems, or 10 delivery systems, with each such delivery system containing a composition comprising a hyper-concentrated hyaluronidase disclosed herein. In aspects of this embodiment, a kit can comprise one or more auto-injecting devices, e.g., 1 to 20 auto-injecting devices, 7 to 15 auto-injecting devices, 8 to 12 auto-injecting devices, or 10 auto-injecting devices, with each such auto-injecting device containing a composition comprising a hyper-concentrated hyaluronidase disclosed herein. In aspects of this embodiment, a kit can comprise one or more manual injecting devices, e.g., 1 to 20 manual injecting devices, 7 to 15 manual injecting devices, 8 to 12 manual injecting devices, or 10 manual injecting devices, with each such manual injecting device containing a composition comprising a hyper-concentrated hyaluronidase disclosed herein.

A kit disclosed herein can comprise other components. For example, a kit disclosed herein can further include containers comprising a solvent, such as, e.g., water or a buffered solution, e.g., saline. A solvent disclosed herein is useful to reconstitute a dried pharmaceutical composition disclosed herein.

A kit disclosed herein can comprise a delivery system. The delivery system of the kit is useful for applying a composition disclosed herein to a site of interest. A delivery or application system disclosed herein includes, without limitation, one or more manual and/or auto-injecting devices disclosed herein. In an embodiment, a kit comprises a single delivery system. In another embodiment, a kit comprises multiple delivery systems. For example, each kit can comprise 2, 3, 4, 5, 6, 7, 8, 9, 10, 11, 12, 13, 14, 15, 16, 17, 18, 19 or 20 or more delivery systems. Within the kit, the delivery system may be packaged individually or in sets of 2 or more. The delivery system can be packaged such that it remains sterile until use. In certain embodiments, a delivery system disclosed herein can be packaged in plastic sheaths. Further, to prevent contamination, a delivery system disclosed herein is preferably a single-use, disposable delivery system.

The kit can also comprise a set of instructions. The instructions may include information useful to the end user such as, e.g., how to use a delivery system to apply a composition disclosed herein and/or how often to apply a composition disclosed herein. In addition, such instructions may include information regarding how to mix a solvent disclosed herein to reconstitute a dried composition disclosed herein. Such instructions can indicate that mixing should be done at a certain time before application, e.g., just prior to use. Instructions disclosed herein may also include information regarding how to apply a composition disclosed herein directly to a site of interest, and in what order or timing the composition disclosed herein should be applied to such sites of interest.

The contents of the kit, including a container, a composition, a delivery system, and instructions disclosed herein, are enclosed in an outer casing. The outer casing can be a box, a sealed bag, a foil pouch, etc. In certain embodiments, the delivery system, container and instructions are enclosed in a box. In other embodiments of the kit, the container and instructions are contained in a first box, the delivery system is contained in a second box, and the first and second boxes are contained together in a third box.

Aspects of the present specification can also be described according to the following embodiments:
1. A method of reducing or eliminating a hyaluronic acid-induced blockage in one or more blood vessels supplying blood to an area of tissue in an individual in need thereof, the method comprising administering a composition comprising a hyper-concentrated hyaluronidase to the individual in a manner that reduces or eliminates a hyaluronic acid-induced blockage in one or more blood vessels supplying blood to an area of tissue being affected by the blockage.
2. A method of reducing or inhibiting a vascular occlusion in an area of tissue of an individual in need thereof, the method comprising administering a composition comprising a hyper-concentrated hyaluronidase to the individual in a manner that reduces or eliminates a hyaluronic acid-induced blockage in one or more blood vessels supplying blood to an area of tissue being affected by the blockage, thereby reducing or inhibiting the vascular occlusion in the area of tissue.
3. A method of reducing or inhibiting a hyaluronic acid-induced loss of tissue in an individual in need thereof, the method comprising administering a composition comprising a hyper-concentrated hyaluronidase to an individual in a manner that reduces or eliminates a hyaluronic acid-induced blockage in one or more blood vessels supplying blood to an area of tissue being affected by the blockage, thereby reducing or inhibiting the hyaluronic acid-induced loss of tissue in the individual.
4. The method according to any one of embodiments 1-3, wherein the composition is administered to a dermal portion of a skin region.
5. The method according to any one of embodiments 1-4, wherein the composition is administered using a delivery system.
6. The method according to any one of embodiments 1-5, wherein the composition comprises the hyper-concentrated hyaluronidase in a concentration of at least 25,000 IU/mL, at least 50,000 IU/mL, at least 75,000 IU/mL, at least 100,000 IU/mL, at least 125,000 IU/mL, at least 150,000 IU/mL, at least 175,000 IU/mL, at least 200,000 IU/mL, at least 225,000 IU/mL, at least 250,000 IU/mL, at least 300,000 IU/mL, at least 350,000 IU/mL, at least 400,000 IU/mL, at least 450,000 IU/mL, or at least 500,000 IU/mL; and/or in a concentration of at most 25,000 IU/mL, at most 50,000 IU/mL, at most 75,000 IU/mL, at most 100,000 IU/mL, at most 125,000 IU/mL, at most 150,000 IU/mL, at most 175,000 IU/mL, at most 200,000 IU/mL, at most 225,000 IU/mL, at most 250,000 IU/mL, at most 300,000 IU/mL, at most 350,000 IU/mL, at most 400,000 IU/mL, at most 450,000 IU/mL, or at most 500,000 IU/mL; or in a concentration of about 25,000 IU/mL to about 50,000 IU/mL, about 25,000 IU/mL to about 75,000 IU/mL, about 25,000 IU/mL to about 100,000 IU/mL, about 25,000 IU/mL to about 125,000 IU/mL, about 25,000 IU/mL to about 150,000 IU/mL, about 25,000 IU/mL to about 175,000 IU/mL, about 25,000 IU/mL to about 200,000 IU/mL, about 25,000 IU/mL to about 225,000 IU/mL, about 25,000 IU/mL to about 250,000 IU/mL, about 25,000 IU/mL to about 300,000 IU/mL, about 25,000 IU/mL to about 350,000 IU/mL, about 25,000 IU/mL to about 400,000 IU/mL, about 25,000 IU/mL to about 450,000 IU/mL, about 25,000 IU/mL to about 500,000 IU/mL, about 50,000 IU/mL to about 75,000 IU/mL, about 50,000 IU/mL to about 100,000 IU/mL, about 50,000 IU/mL to about 125,000 IU/mL, about 50,000 IU/mL to about 150,000 IU/mL, about 50,000 IU/mL to about 175,000 IU/mL, about 50,000 IU/mL to about 200,000 IU/mL, about 50,000 IU/mL to about 225,000 IU/mL, about 50,000 IU/mL to about 250,000 IU/mL, about 50,000 IU/mL to about 300,000 IU/mL, about 50,000 IU/mL to about 350,000 IU/mL, about 50,000 IU/mL to about 400,000 IU/mL, about 50,000 IU/mL to about 450,000 IU/mL, about 50,000 IU/mL to about 500,000 IU/mL, about 75,000 IU/mL to about 100,000 IU/mL, about 75,000 IU/mL to about 125,000 IU/mL, about 75,000 IU/mL to about 150,000 IU/mL, about 75,000 IU/mL to about 175,000 IU/mL, about 75,000 IU/mL to about 200,000 IU/mL, about 75,000 IU/mL to about 225,000 IU/mL, about 75,000 IU/mL to about 250,000 IU/mL, about 75,000 IU/mL to about 300,000 IU/mL, about 75,000 IU/mL to about 350,000 IU/mL, about 75,000 IU/mL to about 400,000 IU/mL, about 75,000 IU/mL to about 450,000 IU/mL, about 75,000 IU/mL to about 500,000 IU/mL, about 100,000 IU/mL to about 125,000 IU/mL, about 100,000 IU/mL to about 150,000 IU/mL, about 100,000 IU/mL to about 175,000 IU/mL, about 100,000 IU/mL to about 200,000 IU/mL, about 100,000 IU/mL to about 225,000 IU/mL, about 100,000 IU/mL to about 250,000 IU/mL, about 100,000 IU/mL to about 300,000 IU/mL, about 100,000 IU/mL to about 350,000 IU/mL, about 100,000 IU/mL to about 400,000 IU/mL, about 100,000 IU/mL to about 450,000 IU/mL, about 100,000 IU/mL to about 500,000 IU/mL, about 150,000 IU/mL to about 200,000 IU/mL, about 150,000 IU/mL to about 225,000 IU/mL, about 150,000 IU/mL to about 250,000 IU/mL, about 150,000 IU/mL to about 300,000 IU/mL, about 150,000 IU/mL to about 350,000 IU/mL, about 150,000 IU/mL to about 400,000 IU/mL, about 150,000 IU/mL to about 450,000 IU/mL, about 150,000 IU/mL to about 500,000 IU/mL, about 200,000 IU/mL to about 225,000 IU/mL, about 200,000 IU/mL to about 250,000 IU/mL, about 200,000 IU/mL to about 300,000 IU/mL, about 200,000 IU/mL to about 350,000 IU/mL, about 200,000 IU/mL to about 400,000 IU/mL, about 200,000 IU/mL to about 450,000 IU/mL, about 200,000 IU/mL to about 500,000 IU/mL, about 250,000 IU/mL to about 300,000 IU/mL, about 250,000 IU/mL to about 350,000 IU/mL, about 250,000 IU/mL to about 400,000 IU/mL, about 250,000 IU/mL to about 450,000 IU/mL, about 250,000 IU/mL to about 500,000 IU/mL, about 300,000 IU/mL to about 350,000 IU/mL, about 300,000 IU/mL to about 400,000 IU/mL, about 300,000 IU/mL to about 450,000 IU/mL, about 300,000 IU/mL to about 500,000 IU/mL, about 350,000 IU/mL to about 400,000 IU/mL, about 350,000 IU/mL to about 450,000 IU/mL, about 350,000 IU/mL to about 500,000 IU/mL, about 400,000 IU/mL to about 450,000 IU/mL, about 400,000 IU/mL to about 500,000 IU/mL, or about 450,000 IU/mL to about 500,000 IU/mL.
7. The method according to any one of embodiments 1-5, wherein the composition comprises the hyper-concentrated hyaluronidase in a concentration of at least 10,000 IU/mL, at least 20,000 IU/mL, at least 30,000 IU/mL, at least 40,000 IU/mL, at least 50,000 IU/mL, at least 60,000 IU/mL, at least 70,000 IU/mL, at least 80,000 IU/mL, at least 90,000 IU/mL, or at least 100,000 IU/mL; and/or in a concentration of at most 10,000 IU/mL, at most 20,000 IU/mL, at most 30,000 IU/mL, at most 40,000 IU/mL, at most 50,000 IU/mL, at most 60,000 IU/mL, at most 70,000 IU/mL, at most 80,000 IU/mL, at most 90,000 IU/mL, or at most 100,000 IU/mL; or in a concentration of about 10,000 IU/mL to about 20,000 IU/mL, about 10,000 IU/mL to about 30,000 IU/mL, about 10,000 IU/mL to about 40,000 IU/mL, about 10,000 IU/mL to about 50,000 IU/mL, about 10,000 IU/mL to about 60,000 IU/mL, about 10,000 IU/mL to about 70,000 IU/mL, about 10,000 IU/mL to about 80,000 IU/mL, about 10,000 IU/mL to about 90,000 IU/mL, about 10,000 IU/mL to about 100,000 IU/mL, about 20,000 IU/mL to about 30,000 IU/mL, about 20,000 IU/mL to about 40,000 IU/mL, about 20,000 IU/mL to about 50,000 IU/mL, about 20,000 IU/mL to about 60,000 IU/mL, about 20,000 IU/mL to about 70,000 IU/mL, about 20,000 IU/mL to about 80,000 IU/mL, about 20,000 IU/mL to about 90,000 IU/mL, about 20,000 IU/mL to about 100,000 IU/mL, about 30,000 IU/mL to about 40,000 IU/mL, about 30,000 IU/mL to about 50,000 IU/mL, about 30,000 IU/mL to about 60,000 IU/mL, about 30,000 IU/mL to about 70,000 IU/mL, about 30,000 IU/mL to about 80,000 IU/mL, about 30,000 IU/mL to about 90,000 IU/mL, about 30,000 IU/mL to about 100,000 IU/mL, about 40,000 IU/mL to about 50,000 IU/mL, about 40,000 IU/mL to about 60,000 IU/mL, about 40,000 IU/mL to about 70,000 IU/mL, about 40,000 IU/mL to about 80,000 IU/mL, about 40,000 IU/mL to about 90,000 IU/mL, about 40,000 IU/mL to about 100,000 IU/mL, about 50,000 IU/mL to about 60,000 IU/mL, about 50,000 IU/mL to about 70,000 IU/mL, about 50,000 IU/mL to about 80,000 IU/mL, about 50,000 IU/mL to about 90,000 IU/mL, about 50,000 IU/mL to about 100,000 IU/mL, about 60,000 IU/mL to about 70,000 IU/mL, about 60,000 IU/mL to about 80,000 IU/mL, about 60,000 IU/mL to about 90,000 IU/mL, about 60,000 IU/mL to about 100,000 IU/mL, about 70,000 IU/mL to about 80,000 IU/mL, about 70,000 IU/mL to about 90,000 IU/mL, about 70,000 IU/mL to about 100,000 IU/mL, about 80,000 IU/mL to about 90,000 IU/mL, about 80,000 IU/mL to about 100,000 IU/mL, or about 90,000 IU/mL to about 100,000 IU/mL.
8. The method according to any one of embodiments 1-5, wherein the composition comprises the hyper-concentrated hyaluronidase in a concentration of at least 5,000 IU/mL, at least 6,000 IU/mL, at least 7,000 IU/mL, at least 8,000 IU/mL, at least 9,000 IU/mL, at least 10,000 IU/mL, at least 11,000 IU/mL, at least 12,000 IU/mL, at least 13,000 IU/mL, at least 14,000 IU/mL, at least 15,000 IU/mL, at least 16,000 IU/mL, at least 17,000 IU/mL, at least 18,000 IU/mL, at least 19,000 IU/mL, or at least 20,000 IU/mL; and/or in a concentration of at most 5,000 IU/mL, at most 6,000 IU/mL, at most 7,000 IU/mL, at most 8,000 IU/mL, at most 9,000 IU/mL, at most 10,000 IU/mL, at most 11,000 IU/mL, at most 12,000 IU/mL, at most 13,000 IU/mL, at most 14,000 IU/mL, at most 15,000 IU/mL, at most 16,000 IU/mL, at most 17,000 IU/mL, at most 18,000 IU/mL, at most 19,000 IU/mL, or at most 20,000 IU/mL; or in a concentration of about 5,000 IU/mL to about 6,000 IU/mL, about 5,000 IU/mL to about 7,000 IU/mL, about 5,000 IU/mL to about 8,000 IU/mL, about 5,000 IU/mL to about 9,000 IU/mL, about 5,000 IU/mL to about 10,000 IU/mL, about 5,000 IU/mL to about 11,000 IU/mL, about 5,000 IU/mL to about 12,000 IU/mL, about 5,000 IU/mL to about 13,000 IU/mL, about 5,000 IU/mL to about 14,000 IU/mL, about 5,000 IU/mL to about 15,000 IU/mL, about 5,000 IU/mL to about 16,000 IU/mL, about 5,000 IU/mL to about 17,000 IU/mL, about 5,000 IU/mL to about 18,000 IU/mL, about 5,000 IU/mL to about 19,000 IU/mL, about 5,000 IU/mL to about 20,000 IU/mL, about 6,000 IU/mL to about 7,000 IU/mL, about 6,000 IU/mL to about 8,000 IU/mL, about 6,000 IU/mL to about 9,000 IU/mL, about 6,000 IU/mL to about 10,000 IU/mL, about 6,000 IU/mL to about 11,000 IU/mL, about 6,000 IU/mL to about 12,000 IU/mL, about 6,000 IU/mL to about 13,000 IU/mL, about 6,000 IU/mL to about 14,000 IU/mL, about 6,000 IU/mL to about 15,000 IU/mL, about 6,000 IU/mL to about 16,000 IU/mL, about 6,000 IU/mL to about 17,000 IU/mL, about 6,000 IU/mL to about 18,000 IU/mL, about 6,000 IU/mL to about 19,000 IU/mL, about 6,000 IU/mL to about 20,000 IU/mL, about 7,000 IU/mL to about 8,000 IU/mL, about 7,000 IU/mL to about 9,000 IU/mL, about 7,000 IU/mL to about 10,000 IU/mL, about 7,000 IU/mL to about 11,000 IU/mL, about 7,000 IU/mL to about 12,000 IU/mL, about 7,000 IU/mL to about 13,000 IU/mL, about 7,000 IU/mL to about 14,000 IU/mL, about 7,000 IU/mL to about 15,000 IU/mL, about 7,000 IU/mL to about 16,000 IU/mL, about 7,000 IU/mL to about 17,000 IU/mL, about 7,000 IU/mL to about 18,000 IU/mL, about 7,000 IU/mL to about 19,000 IU/mL, about 7,000 IU/mL to about 20,000 IU/mL, about 8,000 IU/mL to about 9,000 IU/mL, about 8,000 IU/mL to about 10,000 IU/mL, about 8,000 IU/mL to about 11,000 IU/mL, about 8,000 IU/mL to about 12,000 IU/mL, about 8,000 IU/mL to about 13,000 IU/mL, about 8,000 IU/mL to about 14,000 IU/mL, about 8,000 IU/mL to about 15,000 IU/mL, about 8,000 IU/mL to about 16,000 IU/mL, about 8,000 IU/mL to about 17,000 IU/mL, about 8,000 IU/mL to about 18,000 IU/mL, about 8,000 IU/mL to about 19,000 IU/mL, about 8,000 IU/mL to about 20,000 IU/mL, about 9,000 IU/mL to about 10,000 IU/mL, about 9,000 IU/mL to about 11,000 IU/mL, about 9,000 IU/mL to about 12,000 IU/mL, about 9,000 IU/mL to about 13,000 IU/mL, about 9,000 IU/mL to about 14,000 IU/mL, about 9,000 IU/mL to about 15,000 IU/mL, about 9,000 IU/mL to about 16,000 IU/mL, about 9,000 IU/mL to about 17,000 IU/mL, about 9,000 IU/mL to about 18,000 IU/mL, about 9,000 IU/mL to about 19,000 IU/mL, about 9,000 IU/mL to about 20,000 IU/mL, about 10,000 IU/mL to about 11,000 IU/mL, about 10,000 IU/mL to about 12,000 IU/mL, about 10,000 IU/mL to about 13,000 IU/mL, about 10,000 IU/mL to about 14,000 IU/mL, about 10,000 IU/mL to about 15,000 IU/mL, about 10,000 IU/mL to about 16,000 IU/mL, about 10,000 IU/mL to about 17,000 IU/mL, about 10,000 IU/mL to about 18,000 IU/mL, about 10,000 IU/mL to about 19,000 IU/mL, about 10,000 IU/mL to about 20,000 IU/mL, about 11,000 IU/mL to about 12,000 IU/mL, about 11,000 IU/mL to about 13,000 IU/mL, about 11,000 IU/mL to about 14,000 IU/mL, about 11,000 IU/mL to about 15,000 IU/mL, about 11,000 IU/mL to about 16,000 IU/mL, about 11,000 IU/mL to about 17,000 IU/mL, about 11,000 IU/mL to about 18,000 IU/mL, about 11,000 IU/mL to about 19,000 IU/mL, about 11,000 IU/mL to about 20,000 IU/mL, about 12,000 IU/mL to about 13,000 IU/mL, about 12,000 IU/mL to about 14,000 IU/mL, about 12,000 IU/mL to about 15,000 IU/mL, about 12,000 IU/mL to about 16,000 IU/mL, about 12,000 IU/mL to about 17,000 IU/mL, about 12,000 IU/mL to about 18,000 IU/mL, about 12,000 IU/mL to about 19,000 IU/mL, about 12,000 IU/mL to about 20,000 IU/mL, about 13,000 IU/mL to about 14,000 IU/mL, about 13,000 IU/mL to about 15,000 IU/mL, about 13,000 IU/mL to about 16,000 IU/mL, about 13,000 IU/mL to about 17,000 IU/mL, about 13,000 IU/mL to about 18,000 IU/mL, about 13,000 IU/mL to about 19,000 IU/mL, about 13,000 IU/mL to about 20,000 IU/mL, about 14,000 IU/mL to about 15,000 IU/mL, about 14,000 IU/mL to about 16,000 IU/mL, about 14,000 IU/mL to about 17,000 IU/mL, about 14,000 IU/mL to about 18,000 IU/mL, about 14,000 IU/mL to about 19,000 IU/mL, about 14,000 IU/mL to about 20,000 IU/mL, about 15,000 IU/mL to about 16,000 IU/mL, about 15,000 IU/mL to about 17,000 IU/mL, about 15,000 IU/mL to about 18,000 IU/mL, about 15,000 IU/mL to about 19,000 IU/mL, about 15,000 IU/mL to about 20,000 IU/mL, about 16,000 IU/mL to about 17,000 IU/mL, about 16,000 IU/mL to about 18,000 IU/mL, about 16,000 IU/mL to about 19,000 IU/mL, about 16,000 IU/mL to about 20,000 IU/mL, about 17,000 IU/mL to about 18,000 IU/mL, about 17,000 IU/mL to about 19,000 IU/mL, about 17,000 IU/mL to about 20,000 IU/mL, about 18,000 IU/mL to about 19,000 IU/mL, about 18,000 IU/mL to about 20,000 IU/mL, or about 19,000 IU/mL to about 20,000 IU/mL.
9. The method according to any one of embodiments 1-5, wherein the composition comprises the hyper-concentrated hyaluronidase in a concentration of at least 250 IU/mL, at least 500 IU/mL, at least 750 IU/mL, at least 1,000 IU/mL, at least 1,500 IU/mL, at least 2,000 IU/mL, at least 2,500 IU/mL, at least 3,000 IU/mL, at least 3,500 IU/mL, at least 4,000 IU/mL, at least 4,500 IU/mL, or at least 5,000 IU/mL; and/or in a concentration of at most 250 IU/mL, at most 500 IU/mL, at most 750 IU/mL, at most 1,000 IU/mL, at most 1,500 IU/mL, at most 2,000 IU/mL, at most 2,500 IU/mL, at most 3,000 IU/mL, at most 3,500 IU/mL, at most 4,000 IU/mL, at most 4,500 IU/mL, or at most 5,000 IU/mL; or in a concentration of about 250 IU/mL to about 500 IU/mL, about 250 IU/mL to about 750 IU/mL, about 250 IU/mL to about 1,000 IU/mL, about 250 IU/mL to about 1,500 IU/mL, about 250 IU/mL to about 2,000 IU/mL, about 250 IU/mL to about 2,500 IU/mL, about 250 IU/mL to about 3,000 IU/mL, about 250 IU/mL to about 3,500 IU/mL, about 250 IU/mL to about 4,000 IU/mL, about 250 IU/mL to about 4,500 IU/mL, about 250 IU/mL to about 5,000 IU/mL, about 500 IU/mL to about 750 IU/mL, about 500 IU/mL to about 1,000 IU/mL, about 500 IU/mL to about 1,500 IU/mL, about 500 IU/mL to about 2,000 IU/mL, about 500 IU/mL to about 2,500 IU/mL, about 500 IU/mL to about 3,000 IU/mL, about 500 IU/mL to about 3,500 IU/mL, about 500 IU/mL to about 4,000 IU/mL, about 500 IU/mL to about 4,500 IU/mL, about 500 IU/mL to about 5,000 IU/mL, about 750 IU/mL to about 1,000 IU/mL, about 750 IU/mL to about 1,500 IU/mL, about 750 IU/mL to about 2,000 IU/mL, about 750 IU/mL to about 2,500 IU/mL, about 750 IU/mL to about 3,000 IU/mL, about 750 IU/mL to about 3,500 IU/mL, about 750 IU/mL to about 4,000 IU/mL, about 750 IU/mL to about 4,500 IU/mL, about 750 IU/mL to about 5,000 IU/mL, about 1,000 IU/mL to about 1,500 IU/mL, about 1,000 IU/mL to about 2,000 IU/mL, about 1,000 IU/mL to about 2,500 IU/mL, about 1,000 IU/mL to about 3,000 IU/mL, about 1,000 IU/mL to about 3,500 IU/mL, about 1,000 IU/mL to about 4,000 IU/mL, about 1,000 IU/mL to about 4,500 IU/mL, about 1,000 IU/mL to about 5,000 IU/mL, about 1,500 IU/mL to about 2,000 IU/mL, about 1,500 IU/mL to about 2,500 IU/mL, about 1,500 IU/mL to about 3,000 IU/mL, about 1,500 IU/mL to about 3,500 IU/mL, about 1,500 IU/mL to about 4,000 IU/mL, about 1,500 IU/mL to about 4,500 IU/mL, about 1,500 IU/mL to about 5,000 IU/mL, about 2,000 IU/mL to about 2,500 IU/mL, about 2,000 IU/mL to about 3,000 IU/mL, about 2,000 IU/mL to about 3,500 IU/mL, about 2,000 IU/mL to about 4,000 IU/mL, about 2,000 IU/mL to about 4,500 IU/mL, about 2,000 IU/mL to about 5,000 IU/mL, about 2,500 IU/mL to about 3,000 IU/mL, about 2,500 IU/mL to about 3,500 IU/mL, about 2,500 IU/mL to about 4,000 IU/mL, about 2,500 IU/mL to about 4,500 IU/mL, about 2,500 IU/mL to about 5,000 IU/mL, about 3,000 IU/mL to about 3,500 IU/mL, about 3,000 IU/mL to about 4,000 IU/mL, about 3,000 IU/mL to about 4,500 IU/mL, about 3,000 IU/mL to about 5,000 IU/mL, about 3,500 IU/mL to about 4,000 IU/mL, about 3,500 IU/mL to about 4,500 IU/mL, about 3,500 IU/mL to about 5,000 IU/mL, about 4,000 IU/mL to about 4,500 IU/mL, about 4,000 IU/mL to about 5,000 IU/mL, or about 4,500 IU/mL to about 5,000 IU/mL.
10. The method according to any one of embodiments 1-9, wherein the hyper-concentrated hyaluronidase is administered in an amount of at least 25,000 IU/mL, at least 50,000 IU/mL, at least 75,000 IU/mL, at least 100,000 IU/mL, at least 125,000 IU/mL, at least 150,000 IU/mL, at least 175,000 IU/mL, at least 200,000 IU/mL, at least 225,000 IU/mL, at least 250,000 IU/mL, at least 300,000 IU/mL, at least 350,000 IU/mL, at least 400,000 IU/mL, at least 450,000 IU/mL, or at least 500,000 IU/mL; and/or in an amount of at most 25,000 IU/mL, at most 50,000 IU/mL, at most 75,000 IU/mL, at most 100,000 IU/mL, at most 125,000 IU/mL, at most 150,000 IU/mL, at most 175,000 IU/mL, at most 200,000 IU/mL, at most 225,000 IU/mL, at most 250,000 IU/mL, at most 300,000 IU/mL, at most 350,000 IU/mL, at most 400,000 IU/mL, at most 450,000 IU/mL, or at most 500,000 IU/mL; or in an amount of about 25,000 IU/mL to about 50,000 IU/mL, about 25,000 IU/mL to about 75,000 IU/mL, about 25,000 IU/mL to about 100,000 IU/mL, about 25,000 IU/mL to about 125,000 IU/mL, about 25,000 IU/mL to about 150,000 IU/mL, about 25,000 IU/mL to about 175,000 IU/mL, about 25,000 IU/mL to about 200,000 IU/mL, about 25,000 IU/mL to about 225,000 IU/mL, about 25,000 IU/mL to about 250,000 IU/mL, about 25,000 IU/mL to about 300,000 IU/mL, about 25,000 IU/mL to about 350,000 IU/mL, about 25,000 IU/mL to about 400,000 IU/mL, about 25,000 IU/mL to about 450,000 IU/mL, about 25,000 IU/mL to about 500,000 IU/mL, about 50,000 IU/mL to about 75,000 IU/mL, about 50,000 IU/mL to about 100,000 IU/mL, about 50,000 IU/mL to about 125,000 IU/mL, about 50,000 IU/mL to about 150,000 IU/mL, about 50,000 IU/mL to about 175,000 IU/mL, about 50,000 IU/mL to about 200,000 IU/mL, about 50,000 IU/mL to about 225,000 IU/mL, about 50,000 IU/mL to about 250,000 IU/mL, about 50,000 IU/mL to about 300,000 IU/mL, about 50,000 IU/mL to about 350,000 IU/mL, about 50,000 IU/mL to about 400,000 IU/mL, about 50,000 IU/mL to about 450,000 IU/mL, about 50,000 IU/mL to about 500,000 IU/mL, about 75,000 IU/mL to about 100,000 IU/mL, about 75,000 IU/mL to about 125,000 IU/mL, about 75,000 IU/mL to about 150,000 IU/mL, about 75,000 IU/mL to about 175,000 IU/mL, about 75,000 IU/mL to about 200,000 IU/mL, about 75,000 IU/mL to about 225,000 IU/mL, about 75,000 IU/mL to about 250,000 IU/mL, about 75,000 IU/mL to about 300,000 IU/mL, about 75,000 IU/mL to about 350,000 IU/mL, about 75,000 IU/mL to about 400,000 IU/mL, about 75,000 IU/mL to about 450,000 IU/mL, about 75,000 IU/mL to about 500,000 IU/mL, about 100,000 IU/mL to about 125,000 IU/mL, about 100,000 IU/mL to about 150,000 IU/mL, about 100,000 IU/mL to about 175,000 IU/mL, about 100,000 IU/mL to about 200,000 IU/mL, about 100,000 IU/mL to about 225,000 IU/mL, about 100,000 IU/mL to about 250,000 IU/mL, about 100,000 IU/mL to about 300,000 IU/mL, about 100,000 IU/mL to about 350,000 IU/mL, about 100,000 IU/mL to about 400,000 IU/mL, about 100,000 IU/mL to about 450,000 IU/mL, about 100,000 IU/mL to about 500,000 IU/mL, about 150,000 IU/mL to about 200,000 IU/mL, about 150,000 IU/mL to about 225,000 IU/mL, about 150,000 IU/mL to about 250,000 IU/mL, about 150,000 IU/mL to about 300,000 IU/mL, about 150,000 IU/mL to about 350,000 IU/mL, about 150,000 IU/mL to about 400,000 IU/mL, about 150,000 IU/mL to about 450,000 IU/mL, about 150,000 IU/mL to about 500,000 IU/mL, about 200,000 IU/mL to about 225,000 IU/mL, about 200,000 IU/mL to about 250,000 IU/mL, about 200,000 IU/mL to about 300,000 IU/mL, about 200,000 IU/mL to about 350,000 IU/mL, about 200,000 IU/mL to about 400,000 IU/mL, about 200,000 IU/mL to about 450,000 IU/mL, about 200,000 IU/mL to about 500,000 IU/mL, about 250,000 IU/mL to about 300,000 IU/mL, about 250,000 IU/mL to about 350,000 IU/mL, about 250,000 IU/mL to about 400,000 IU/mL, about 250,000 IU/mL to about 450,000 IU/mL, about 250,000 IU/mL to about 500,000 IU/mL, about 300,000 IU/mL to about 350,000 IU/mL, about 300,000 IU/mL to about 400,000 IU/mL, about 300,000 IU/mL to about 450,000 IU/mL, about 300,000 IU/mL to about 500,000 IU/mL, about 350,000 IU/mL to about 400,000 IU/mL, about 350,000 IU/mL to about 450,000 IU/mL, about 350,000 IU/mL to about 500,000 IU/mL, about 400,000 IU/mL to about 450,000 IU/mL, about 400,000 IU/mL to about 500,000 IU/mL, or about 450,000 IU/mL to about 500,000 IU/mL.
11. The method according to any one of embodiments 1-9, wherein the hyper-concentrated hyaluronidase is administered in an amount of at least 10,000 IU/mL, at least 20,000 IU/mL, at least 30,000 IU/mL, at least 40,000 IU/mL, at least 50,000 IU/mL, at least 60,000 IU/mL, at least 70,000 IU/mL, at least 80,000 IU/mL, at least 90,000 IU/mL, or at least 100,000 IU/mL; and/or in an amount of at most 10,000 IU/mL, at most 20,000 IU/mL, at most 30,000 IU/mL, at most 40,000 IU/mL, at most 50,000 IU/mL, at most 60,000 IU/mL, at most 70,000 IU/mL, at most 80,000 IU/mL, at most 90,000 IU/mL, or at most 100,000 IU/mL; or in an amount of about 10,000 IU/mL to about 20,000 IU/mL, about 10,000 IU/mL to about 30,000 IU/mL, about 10,000 IU/mL to about 40,000 IU/mL, about 10,000 IU/mL to about 50,000 IU/mL, about 10,000 IU/mL to about 60,000 IU/mL, about 10,000 IU/mL to about 70,000 IU/mL, about 10,000 IU/mL to about 80,000 IU/mL, about 10,000 IU/mL to about 90,000 IU/mL, about 10,000 IU/mL to about 100,000 IU/mL, about 20,000 IU/mL to about 30,000 IU/mL, about 20,000 IU/mL to about 40,000 IU/mL, about 20,000 IU/mL to about 50,000 IU/mL, about 20,000 IU/mL to about 60,000 IU/mL, about 20,000 IU/mL to about 70,000 IU/mL, about 20,000 IU/mL to about 80,000 IU/mL, about 20,000 IU/mL to about 90,000 IU/mL, about 20,000 IU/mL to about 100,000 IU/mL, about 30,000 IU/mL to about 40,000 IU/mL, about 30,000 IU/mL to about 50,000 IU/mL, about 30,000 IU/mL to about 60,000 IU/mL, about 30,000 IU/mL to about 70,000 IU/mL, about 30,000 IU/mL to about 80,000 IU/mL, about 30,000 IU/mL to about 90,000 IU/mL, about 30,000 IU/mL to about 100,000 IU/mL, about 40,000 IU/mL to about 50,000 IU/mL, about 40,000 IU/mL to about 60,000 IU/mL, about 40,000 IU/mL to about 70,000 IU/mL, about 40,000 IU/mL to about 80,000 IU/mL, about 40,000 IU/mL to about 90,000 IU/mL, about 40,000 IU/mL to about 100,000 IU/mL, about 50,000 IU/mL to about 60,000 IU/mL, about 50,000 IU/mL to about 70,000 IU/mL, about 50,000 IU/mL to about 80,000 IU/mL, about 50,000 IU/mL to about 90,000 IU/mL, about 50,000 IU/mL to about 100,000 IU/mL, about 60,000 IU/mL to about 70,000 IU/mL, about 60,000 IU/mL to about 80,000 IU/mL, about 60,000 IU/mL to about 90,000 IU/mL, about 60,000 IU/mL to about 100,000 IU/mL, about 70,000 IU/mL to about 80,000 IU/mL, about 70,000 IU/mL to about 90,000 IU/mL, about 70,000 IU/mL to about 100,000 IU/mL, about 80,000 IU/mL to about 90,000 IU/mL, about 80,000 IU/mL to about 100,000 IU/mL, or about 90,000 IU/mL to about 100,000 IU/mL.
12. The method according to any one of embodiments 1-9, wherein the hyper-concentrated hyaluronidase is administered in an amount of at least 10,000 IU/mL, at least 11,000 IU/mL, at least 12,000 IU/mL, at least 13,000 IU/mL, at least 14,000 IU/mL, at least 15,000 IU/mL, at least 16,000 IU/mL, at least 17,000 IU/mL, at least 18,000 IU/mL, at least 19,000 IU/mL, or at least 20,000 IU/mL; and/or in an amount of at most 10,000 IU/mL, at most 11,000 IU/mL, at most 12,000 IU/mL, at most 13,000 IU/mL, at most 14,000 IU/mL, at most 15,000 IU/mL, at most 16,000 IU/mL, at most 17,000 IU/mL, at most 18,000 IU/mL, at most 19,000 IU/mL, or at most 20,000 IU/mL; or in an amount of about 10,000 IU/mL to about 11,000 IU/mL, about 10,000 IU/mL to about 12,000 IU/mL, about 10,000 IU/mL to about 13,000 IU/mL, about 10,000 IU/mL to about 14,000 IU/mL, about 10,000 IU/mL to about 15,000 IU/mL, about 10,000 IU/mL to about 16,000 IU/mL, about 10,000 IU/mL to about 17,000 IU/mL, about 10,000 IU/mL to about 18,000 IU/mL, about 10,000 IU/mL to about 19,000 IU/mL, about 10,000 IU/mL to about 20,000 IU/mL, about 11,000 IU/mL to about 12,000 IU/mL, about 11,000 IU/mL to about 13,000 IU/mL, about 11,000 IU/mL to about 14,000 IU/mL, about 11,000 IU/mL to about 15,000 IU/mL, about 11,000 IU/mL to about 16,000 IU/mL, about 11,000 IU/mL to about 17,000 IU/mL, about 11,000 IU/mL to about 18,000 IU/mL, about 11,000 IU/mL to about 19,000 IU/mL, about 11,000 IU/mL to about 20,000 IU/mL, about 12,000 IU/mL to about 13,000 IU/mL, about 12,000 IU/mL to about 14,000 IU/mL, about 12,000 IU/mL to about 15,000 IU/mL, about 12,000 IU/mL to about 16,000 IU/mL, about 12,000 IU/mL to about 17,000 IU/mL, about 12,000 IU/mL to about 18,000 IU/mL, about 12,000 IU/mL to about 19,000 IU/mL, about 12,000 IU/mL to about 20,000 IU/mL, about 13,000 IU/mL to about 14,000 IU/mL, about 13,000 IU/mL to about 15,000 IU/mL, about 13,000 IU/mL to about 16,000 IU/mL, about 13,000 IU/mL to about 17,000 IU/mL, about 13,000 IU/mL to about 18,000 IU/mL, about 13,000 IU/mL to about 19,000 IU/mL, about 13,000 IU/mL to about 20,000 IU/mL, about 14,000 IU/mL to about 15,000 IU/mL, about 14,000 IU/mL to about 16,000 IU/mL, about 14,000 IU/mL to about 17,000 IU/mL, about 14,000 IU/mL to about 18,000 IU/mL, about 14,000 IU/mL to about 19,000 IU/mL, about 14,000 IU/mL to about 20,000 IU/mL, about 15,000 IU/mL to about 16,000 IU/mL, about 15,000 IU/mL to about 17,000 IU/mL, about 15,000 IU/mL to about 18,000 IU/mL, about 15,000 IU/mL to about 19,000 IU/mL, about 15,000 IU/mL to about 20,000 IU/mL, about 16,000 IU/mL to about 17,000 IU/mL, about 16,000 IU/mL to about 18,000 IU/mL, about 16,000 IU/mL to about 19,000 IU/mL, about 16,000 IU/mL to about 20,000 IU/mL, about 17,000 IU/mL to about 18,000 IU/mL, about 17,000 IU/mL to about 19,000 IU/mL, about 17,000 IU/mL to about 20,000 IU/mL, about 18,000 IU/mL to about 19,000 IU/mL, about 18,000 IU/mL to about 20,000 IU/mL, or about 19,000 IU/mL to about 20,000 IU/mL.
13. The method according to any one of embodiments 1-9, wherein the hyper-concentrated hyaluronidase is administered in an amount of at least 250 IU/mL, at least 500 IU/mL, at least 750 IU/mL, at least 1,000 IU/mL, at least 1,500 IU/mL, at least 2,000 IU/mL, at least 2,500 IU/mL, at least 3,000 IU/mL, at least 3,500 IU/mL, at least 4,000 IU/mL, at least 4,500 IU/mL, or at least 5,000 IU/mL; and/or in an amount of at most 250 IU/mL, at most 500 IU/mL, at most 750 IU/mL, at most 1,000 IU/mL, at most 1,500 IU/mL, at most 2,000 IU/mL, at most 2,500 IU/mL, at most 3,000 IU/mL, at most 3,500 IU/mL, at most 4,000 IU/mL, at most 4,500 IU/mL, or at most 5,000 IU/mL; or in an amount of about 250 IU/mL to about 500 IU/mL, about 250 IU/mL to about 750 IU/mL, about 250 IU/mL to about 1,000 IU/mL, about 250 IU/mL to about 1,500 IU/mL, about 250 IU/mL to about 2,000 IU/mL, about 250 IU/mL to about 2,500 IU/mL, about 250 IU/mL to about 3,000 IU/mL, about 250 IU/mL to about 3,500 IU/mL, about 250 IU/mL to about 4,000 IU/mL, about 250 IU/mL to about 4,500 IU/mL, about 250 IU/mL to about 5,000 IU/mL, about 500 IU/mL to about 750 IU/mL, about 500 IU/mL to about 1,000 IU/mL, about 500 IU/mL to about 1,500 IU/mL, about 500 IU/mL to about 2,000 IU/mL, about 500 IU/mL to about 2,500 IU/mL, about 500 IU/mL to about 3,000 IU/mL, about 500 IU/mL to about 3,500 IU/mL, about 500 IU/mL to about 4,000 IU/mL, about 500 IU/mL to about 4,500 IU/mL, about 500 IU/mL to about 5,000 IU/mL, about 750 IU/mL to about 1,000 IU/mL, about 750 IU/mL to about 1,500 IU/mL, about 750 IU/mL to about 2,000 IU/mL, about 750 IU/mL to about 2,500 IU/mL, about 750 IU/mL to about 3,000 IU/mL, about 750 IU/mL to about 3,500 IU/mL, about 750 IU/mL to about 4,000 IU/mL, about 750 IU/mL to about 4,500 IU/mL, about 750 IU/mL to about 5,000 IU/mL, about 1,000 IU/mL to about 1,500 IU/mL, about 1,000 IU/mL to about 2,000 IU/mL, about 1,000 IU/mL to about 2,500 IU/mL, about 1,000 IU/mL to about 3,000 IU/mL, about 1,000 IU/mL to about 3,500 IU/mL, about 1,000 IU/mL to about 4,000 IU/mL, about 1,000 IU/mL to about 4,500 IU/mL, about 1,000 IU/mL to about 5,000 IU/mL, about 1,500 IU/mL to about 2,000 IU/mL, about 1,500 IU/mL to about 2,500 IU/mL, about 1,500 IU/mL to about 3,000 IU/mL, about 1,500 IU/mL to about 3,500 IU/mL, about 1,500 IU/mL to about 4,000 IU/mL, about 1,500 IU/mL to about 4,500 IU/mL, about 1,500 IU/mL to about 5,000 IU/mL, about 2,000 IU/mL to about 2,500 IU/mL, about 2,000 IU/mL to about 3,000 IU/mL, about 2,000 IU/mL to about 3,500 IU/mL, about 2,000 IU/mL to about 4,000 IU/mL, about 2,000 IU/mL to about 4,500 IU/mL, about 2,000 IU/mL to about 5,000 IU/mL, about 2,500 IU/mL to about 3,000 IU/mL, about 2,500 IU/mL to about 3,500 IU/mL, about 2,500 IU/mL to about 4,000 IU/mL, about 2,500 IU/mL to about 4,500 IU/mL, about 2,500 IU/mL to about 5,000 IU/mL, about 3,000 IU/mL to about 3,500 IU/mL, about 3,000 IU/mL to about 4,000 IU/mL, about 3,000 IU/mL to about 4,500 IU/mL, about 3,000 IU/mL to about 5,000 IU/mL, about 3,500 IU/mL to about 4,000 IU/mL, about 3,500 IU/mL to about 4,500 IU/mL, about 3,500 IU/mL to about 5,000 IU/mL, about 4,000 IU/mL to about 4,500 IU/mL, about 4,000 IU/mL to about 5,000 IU/mL, or about 4,500 IU/mL to about 5,000 IU/mL.
14. The method according to any one of embodiments 1-13, wherein the composition is administered in a single dose or in multiple doses.
15. The method according to any one of embodiments 1-14, wherein the composition is administered in a volume of at least 0.5 mL, at least 1.0 mL, at least 2.0 mL, at least 3.0 mL, at least 4.0 mL, or at least 5.0 mL; and/or in a volume of at most 0.5 mL, at most 1.0 mL, at most 2.0 mL, at most 3.0 mL, at most 4.0 mL, or at most 5.0 mL; or in a volume of about 0.5 mL to 1.0 mL, about 0.5 mL to 2.0 mL, about 0.5 mL to 3.0 mL, about 0.5 mL to 4.0 mL, about 0.5 mL to 5.0 mL, about 1.0 mL to 2.0 mL, about 1.0 mL to 3.0 mL, about 1.0 mL to 4.0 mL, about 1.0 to 5.0 mL, about 2.0 mL to 3.0 mL, about 2.0 mL to 4.0 mL, about 2.0 to 5.0 mL, about 3.0 mL to 4.0 mL, about 3.0 to 5.0 mL, or about 4.0 to 5.0 mL.
16. The method according to any one of embodiments 1-15, wherein the composition enters the area of tissue in at most 1 minute, at most 2 minutes, at most 3 minutes, at most 4 minutes, at most 5 minutes, at most 10 minutes, at most 15 minutes, at most 30 minutes, at most 45 minutes, at most 60 minutes, at most 75 minutes or at most 90 minutes; or in about 1 minute to about 2 minutes, about 1 minute to about 3 minutes, about 1 minute to about 5 minutes, about 1 minute to about 7 minutes, about 1 minute to about 10 minutes, about 1 minute to about 15 minutes, about 1 minute to about 30 minutes, about 1 minute to about 45 minutes, about 1 minute to about 60 minutes, about 1 minute to about 75 minutes, about 1 minute to about 90 minutes, about 3 minutes to about 5 minutes, about 3 minutes to about 7 minutes, about 3 minutes to about 10 minutes, about 3 minutes to about 15 minutes, about 3 minutes to about 30 minutes, about 3 minutes to about 45 minutes, about 3 minutes to about 60 minutes, about 3 minutes to about 75 minutes, about 3 minutes to about 90 minutes, about 5 minutes to about 7 minutes, about 5 minutes to about 10 minutes, about 5 minutes to about 15 minutes, about 5 minutes to about 30 minutes, about 5 minutes to about 45 minutes, about 5 minutes to about 60 minutes, about 5 minutes to about 75 minutes, about 5 minutes to about 90 minutes, about 10 minutes to about 15 minutes, about 10 minutes to about 30 minutes, about 10 minutes to about 45 minutes, about 10 minutes to about 60 minutes, about 10 minutes to about 75 minutes, about 10 minutes to about 90 minutes, about 15 minutes to about 30 minutes, about 15 minutes to about 45 minutes, about 15 minutes to about 60 minutes, about 15 minutes to about 75 minutes, about 15 minutes to about 90 minutes.
17. The method according to any one of embodiments 1-16, wherein the hyaluronic acid-induced blockage in the one or more blood vessels supplying blood to the area of tissue being affected by the blockage is reduced or eliminated in at most 1 minute, at most 2 minutes, at most 3 minutes, at most 4 minutes, at most 5 minutes, at most 10 minutes, or at most 15 minutes, at most 30 minutes, at most 45 minutes, at most 60 minutes, at most 75 minutes or at most 90 minutes; or in about 1 minute to about 2 minutes, about 1 minute to about 3 minutes, about 1 minute to about 5 minutes, about 1 minute to about 7 minutes, about 1 minute to about 10 minutes, about 1 minute to about 15 minutes, about 1 minute to about 30 minutes, about 1 minute to about 45 minutes, about 1 minute to about 60 minutes, about 1 minute to about 75 minutes, about 1 minute to about 90 minutes, about 3 minutes to about 5 minutes, about 3 minutes to about 7 minutes, about 3 minutes to about 10 minutes, about 3 minutes to about 15 minutes, about 3 minutes to about 30 minutes, about 3 minutes to about 45 minutes, about 3 minutes to about 60 minutes, about 3 minutes to about 75 minutes, about 3 minutes to about 90 minutes, about 5 minutes to about 7 minutes, about 5 minutes to about 10 minutes, about 5 minutes to about 15 minutes, about 5 minutes to about 30 minutes, about 5 minutes to about 45 minutes, about 5 minutes to about 60 minutes, about 5 minutes to about 75 minutes, about 5 minutes to about 90 minutes, about 10 minutes to about 15 minutes, about 10 minutes to about 30 minutes, about 10 minutes to about 45 minutes, about 10 minutes to about 60 minutes, about 10 minutes to about 75 minutes, about 10 minutes to about 90 minutes, about 15 minutes to about 30 minutes, about 15 minutes to about 45 minutes, about 15 minutes to about 60 minutes, about 15 minutes to about 75 minutes, about 15 minutes to about 90 minutes.
18. A composition comprising a hyper-concentrated hyaluronidase for use in reducing or eliminating a hyaluronic acid-induced blockage in the one or more blood vessels supplying blood to an area of tissue.
19. Use of a composition comprising a hyper-concentrated hyaluronidase for reducing or eliminating a hyaluronic acid-induced blockage in the one or more blood vessels supplying blood to an area of tissue.
20. A composition comprising a hyper-concentrated hyaluronidase for use in the manufacture of a medicament for reducing or eliminating a hyaluronic acid-induced blockage in the one or more blood vessels supplying blood to an area of tissue.
21. The use according to any one of embodiments 18-20, wherein the composition is administered using a delivery system.
22. A delivery system comprising a composition including a hyper-concentrated hyaluronidase for use in reducing or eliminating a hyaluronic acid-induced blockage in the one or more blood vessels supplying blood to an area of tissue.
23. Use of a delivery system comprising a composition including a hyper-concentrated hyaluronidase for reducing or eliminating a hyaluronic acid-induced blockage in the one or more blood vessels supplying blood to an area of tissue.
24. A composition comprising a hyper-concentrated hyaluronidase for use in the manufacture of a medicament for reducing or eliminating a hyaluronic acid-induced blockage in the one or more blood vessels supplying blood to an area of tissue, wherein the composition is contained in a delivery system.
25. The use according to any one of embodiments 18-24, wherein the composition is administered to a dermal portion of a skin region.
26. The use according to any one of embodiments 18-25, wherein the composition comprises the hyaluronidase in a concentration of at least 25,000 IU/mL, at least 50,000 IU/mL, at least 75,000 IU/mL, at least 100,000 IU/mL, at least 125,000 IU/mL, at least 150,000 IU/mL, at least 175,000 IU/mL, at least 200,000 IU/mL, at least 225,000 IU/mL, at least 250,000 IU/mL, at least 300,000 IU/mL, at least 350,000 IU/mL, at least 400,000 IU/mL, at least 450,000 IU/mL, or at least 500,000 IU/mL; and/or in a concentration of at most 25,000 IU/mL, at most 50,000 IU/mL, at most 75,000 IU/mL, at most 100,000 IU/mL, at most 125,000 IU/mL, at most 150,000 IU/mL, at most 175,000 IU/mL, at most 200,000 IU/mL, at most 225,000 IU/mL, at most 250,000 IU/mL, at most 300,000 IU/mL, at most 350,000 IU/mL, at most 400,000 IU/mL, at most 450,000 IU/mL, or at most 500,000 IU/mL; or in a concentration of about 25,000 IU/mL to about 50,000 IU/mL, about 25,000 IU/mL to about 75,000 IU/mL, about 25,000 IU/mL to about 100,000 IU/mL, about 25,000 IU/mL to about 125,000 IU/mL, about 25,000 IU/mL to about 150,000 IU/mL, about 25,000 IU/mL to about 175,000 IU/mL, about 25,000 IU/mL to about 200,000 IU/mL, about 25,000 IU/mL to about 225,000 IU/mL, about 25,000 IU/mL to about 250,000 IU/mL, about 25,000 IU/mL to about 300,000 IU/mL, about 25,000 IU/mL to about 350,000 IU/mL, about 25,000 IU/mL to about 400,000 IU/mL, about 25,000 IU/mL to about 450,000 IU/mL, about 25,000 IU/mL to about 500,000 IU/mL, about 50,000 IU/mL to about 75,000 IU/mL, about 50,000 IU/mL to about 100,000 IU/mL, about 50,000 IU/mL to about 125,000 IU/mL, about 50,000 IU/mL to about 150,000 IU/mL, about 50,000 IU/mL to about 175,000 IU/mL, about 50,000 IU/mL to about 200,000 IU/mL, about 50,000 IU/mL to about 225,000 IU/mL, about 50,000 IU/mL to about 250,000 IU/mL, about 50,000 IU/mL to about 300,000 IU/mL, about 50,000 IU/mL to about 350,000 IU/mL, about 50,000 IU/mL to about 400,000 IU/mL, about 50,000 IU/mL to about 450,000 IU/mL, about 50,000 IU/mL to about 500,000 IU/mL, about 75,000 IU/mL to about 100,000 IU/mL, about 75,000 IU/mL to about 125,000 IU/mL, about 75,000 IU/mL to about 150,000 IU/mL, about 75,000 IU/mL to about 175,000 IU/mL, about 75,000 IU/mL to about 200,000 IU/mL, about 75,000 IU/mL to about 225,000 IU/mL, about 75,000 IU/mL to about 250,000 IU/mL, about 75,000 IU/mL to about 300,000 IU/mL, about 75,000 IU/mL to about 350,000 IU/mL, about 75,000 IU/mL to about 400,000 IU/mL, about 75,000 IU/mL to about 450,000 IU/mL, about 75,000 IU/mL to about 500,000 IU/mL, about 100,000 IU/mL to about 125,000 IU/mL, about 100,000 IU/mL to about 150,000 IU/mL, about 100,000 IU/mL to about 175,000 IU/mL, about 100,000 IU/mL to about 200,000 IU/mL, about 100,000 IU/mL to about 225,000 IU/mL, about 100,000 IU/mL to about 250,000 IU/mL, about 100,000 IU/mL to about 300,000 IU/mL, about 100,000 IU/mL to about 350,000 IU/mL, about 100,000 IU/mL to about 400,000 IU/mL, about 100,000 IU/mL to about 450,000 IU/mL, about 100,000 IU/mL to about 500,000 IU/mL, about 150,000 IU/mL to about 200,000 IU/mL, about 150,000 IU/mL to about 225,000 IU/mL, about 150,000 IU/mL to about 250,000 IU/mL, about 150,000 IU/mL to about 300,000 IU/mL, about 150,000 IU/mL to about 350,000 IU/mL, about 150,000 IU/mL to about 400,000 IU/mL, about 150,000 IU/mL to about 450,000 IU/mL, about 150,000 IU/mL to about 500,000 IU/mL, about 200,000 IU/mL to about 225,000 IU/mL, about 200,000 IU/mL to about 250,000 IU/mL, about 200,000 IU/mL to about 300,000 IU/mL, about 200,000 IU/mL to about 350,000 IU/mL, about 200,000 IU/mL to about 400,000 IU/mL, about 200,000 IU/mL to about 450,000 IU/mL, about 200,000 IU/mL to about 500,000 IU/mL, about 250,000 IU/mL to about 300,000 IU/mL, about 250,000 IU/mL to about 350,000 IU/mL, about 250,000 IU/mL to about 400,000 IU/mL, about 250,000 IU/mL to about 450,000 IU/mL, about 250,000 IU/mL to about 500,000 IU/mL, about 300,000 IU/mL to about 350,000 IU/mL, about 300,000 IU/mL to about 400,000 IU/mL, about 300,000 IU/mL to about 450,000 IU/mL, about 300,000 IU/mL to about 500,000 IU/mL, about 350,000 IU/mL to about 400,000 IU/mL, about 350,000 IU/mL to about 450,000 IU/mL, about 350,000 IU/mL to about 500,000 IU/mL, about 400,000 IU/mL to about 450,000 IU/mL, about 400,000 IU/mL to about 500,000 IU/mL, or about 450,000 IU/mL to about 500,000 IU/mL.
27. The use according to any one of embodiments 18-25, wherein the composition comprises the hyaluronidase in a concentration of at least 10,000 IU/mL, at least 20,000 IU/mL, at least 30,000 IU/mL, at least 40,000 IU/mL, at least 50,000 IU/mL, at least 60,000 IU/mL, at least 70,000 IU/mL, at least 80,000 IU/mL, at least 90,000 IU/mL, or at least 100,000 IU/mL; and/or in a concentration of at most 10,000 IU/mL, at most 20,000 IU/mL, at most 30,000 IU/mL, at most 40,000 IU/mL, at most 50,000 IU/mL, at most 60,000 IU/mL, at most 70,000 IU/mL, at most 80,000 IU/mL, at most 90,000 IU/mL, or at most 100,000 IU/mL; or in a concentration of about 10,000 IU/mL to about 20,000 IU/mL, about 10,000 IU/mL to about 30,000 IU/mL, about 10,000 IU/mL to about 40,000 IU/mL, about 10,000 IU/mL to about 50,000 IU/mL, about 10,000 IU/mL to about 60,000 IU/mL, about 10,000 IU/mL to about 70,000 IU/mL, about 10,000 IU/mL to about 80,000 IU/mL, about 10,000 IU/mL to about 90,000 IU/mL, about 10,000 IU/mL to about 100,000 IU/mL, about 20,000 IU/mL to about 30,000 IU/mL, about 20,000 IU/mL to about 40,000 IU/mL, about 20,000 IU/mL to about 50,000 IU/mL, about 20,000 IU/mL to about 60,000 IU/mL, about 20,000 IU/mL to about 70,000 IU/mL, about 20,000 IU/mL to about 80,000 IU/mL, about 20,000 IU/mL to about 90,000 IU/mL, about 20,000 IU/mL to about 100,000 IU/mL, about 30,000 IU/mL to about 40,000 IU/mL, about 30,000 IU/mL to about 50,000 IU/mL, about 30,000 IU/mL to about 60,000 IU/mL, about 30,000 IU/mL to about 70,000 IU/mL, about 30,000 IU/mL to about 80,000 IU/mL, about 30,000 IU/mL to about 90,000 IU/mL, about 30,000 IU/mL to about 100,000 IU/mL, about 40,000 IU/mL to about 50,000 IU/mL, about 40,000 IU/mL to about 60,000 IU/mL, about 40,000 IU/mL to about 70,000 IU/mL, about 40,000 IU/mL to about 80,000 IU/mL, about 40,000 IU/mL to about 90,000 IU/mL, about 40,000 IU/mL to about 100,000 IU/mL, about 50,000 IU/mL to about 60,000 IU/mL, about 50,000 IU/mL to about 70,000 IU/mL, about 50,000 IU/mL to about 80,000 IU/mL, about 50,000 IU/mL to about 90,000 IU/mL, about 50,000 IU/mL to about 100,000 IU/mL, about 60,000 IU/mL to about 70,000 IU/mL, about 60,000 IU/mL to about 80,000 IU/mL, about 60,000 IU/mL to about 90,000 IU/mL, about 60,000 IU/mL to about 100,000 IU/mL, about 70,000 IU/mL to about 80,000 IU/mL, about 70,000 IU/mL to about 90,000 IU/mL, about 70,000 IU/mL to about 100,000 IU/mL, about 80,000 IU/mL to about 90,000 IU/mL, about 80,000 IU/mL to about 100,000 IU/mL, or about 90,000 IU/mL to about 100,000 IU/mL.
28. The use according to any one of embodiments 18-25, wherein the composition comprises the hyaluronidase in a concentration of at least 5,000 IU/mL, at least 6,000 IU/mL, at least 7,000 IU/mL, at least 8,000 IU/mL, at least 9,000 IU/mL, at least 10,000 IU/mL, at least 11,000 IU/mL, at least 12,000 IU/mL, at least 13,000 IU/mL, at least 14,000 IU/mL, at least 15,000 IU/mL, at least 16,000 IU/mL, at least 17,000 IU/mL, at least 18,000 IU/mL, at least 19,000 IU/mL, or at least 20,000 IU/mL; and/or in a concentration of at most 5,000 IU/mL, at most 6,000 IU/mL, at most 7,000 IU/mL, at most 8,000 IU/mL, at most 9,000 IU/mL, at most 10,000 IU/mL, at most 11,000 IU/mL, at most 12,000 IU/mL, at most 13,000 IU/mL, at most 14,000 IU/mL, at most 15,000 IU/mL, at most 16,000 IU/mL, at most 17,000 IU/mL, at most 18,000 IU/mL, at most 19,000 IU/mL, or at most 20,000 IU/mL; or in a concentration of about 5,000 IU/mL to about 6,000 IU/mL, about 5,000 IU/mL to about 7,000 IU/mL, about 5,000 IU/mL to about 8,000 IU/mL, about 5,000 IU/mL to about 9,000 IU/mL, about 5,000 IU/mL to about 10,000 IU/mL, about 5,000 IU/mL to about 11,000 IU/mL, about 5,000 IU/mL to about 12,000 IU/mL, about 5,000 IU/mL to about 13,000 IU/mL, about 5,000 IU/mL to about 14,000 IU/mL, about 5,000 IU/mL to about 15,000 IU/mL, about 5,000 IU/mL to about 16,000 IU/mL, about 5,000 IU/mL to about 17,000 IU/mL, about 5,000 IU/mL to about 18,000 IU/mL, about 5,000 IU/mL to about 19,000 IU/mL, about 5,000 IU/mL to about 20,000 IU/mL, about 6,000 IU/mL to about 7,000 IU/mL, about 6,000 IU/mL to about 8,000 IU/mL, about 6,000 IU/mL to about 9,000 IU/mL, about 6,000 IU/mL to about 10,000 IU/mL, about 6,000 IU/mL to about 11,000 IU/mL, about 6,000 IU/mL to about 12,000 IU/mL, about 6,000 IU/mL to about 13,000 IU/mL, about 6,000 IU/mL to about 14,000 IU/mL, about 6,000 IU/mL to about 15,000 IU/mL, about 6,000 IU/mL to about 16,000 IU/mL, about 6,000 IU/mL to about 17,000 IU/mL, about 6,000 IU/mL to about 18,000 IU/mL, about 6,000 IU/mL to about 19,000 IU/mL, about 6,000 IU/mL to about 20,000 IU/mL, about 7,000 IU/mL to about 8,000 IU/mL, about 7,000 IU/mL to about 9,000 IU/mL, about 7,000 IU/mL to about 10,000 IU/mL, about 7,000 IU/mL to about 11,000 IU/mL, about 7,000 IU/mL to about 12,000 IU/mL, about 7,000 IU/mL to about 13,000 IU/mL, about 7,000 IU/mL to about 14,000 IU/mL, about 7,000 IU/mL to about 15,000 IU/mL, about 7,000 IU/mL to about 16,000 IU/mL, about 7,000 IU/mL to about 17,000 IU/mL, about 7,000 IU/mL to about 18,000 IU/mL, about 7,000 IU/mL to about 19,000 IU/mL, about 7,000 IU/mL to about 20,000 IU/mL, about 8,000 IU/mL to about 9,000 IU/mL, about 8,000 IU/mL to about 10,000 IU/mL, about 8,000 IU/mL to about 11,000 IU/mL, about 8,000 IU/mL to about 12,000 IU/mL, about 8,000 IU/mL to about 13,000 IU/mL, about 8,000 IU/mL to about 14,000 IU/mL, about 8,000 IU/mL to about 15,000 IU/mL, about 8,000 IU/mL to about 16,000 IU/mL, about 8,000 IU/mL to about 17,000 IU/mL, about 8,000 IU/mL to about 18,000 IU/mL, about 8,000 IU/mL to about 19,000 IU/mL, about 8,000 IU/mL to about 20,000 IU/mL, about 9,000 IU/mL to about 10,000 IU/mL, about 9,000 IU/mL to about 11,000 IU/mL, about 9,000 IU/mL to about 12,000 IU/mL, about 9,000 IU/mL to about 13,000 IU/mL, about 9,000 IU/mL to about 14,000 IU/mL, about 9,000 IU/mL to about 15,000 IU/mL, about 9,000 IU/mL to about 16,000 IU/mL, about 9,000 IU/mL to about 17,000 IU/mL, about 9,000 IU/mL to about 18,000 IU/mL, about 9,000 IU/mL to about 19,000 IU/mL, about 9,000 IU/mL to about 20,000 IU/mL, about 10,000 IU/mL to about 11,000 IU/mL, about 10,000 IU/mL to about 12,000 IU/mL, about 10,000 IU/mL to about 13,000 IU/mL, about 10,000 IU/mL to about 14,000 IU/mL, about 10,000 IU/mL to about 15,000 IU/mL, about 10,000 IU/mL to about 16,000 IU/mL, about 10,000 IU/mL to about 17,000 IU/mL, about 10,000 IU/mL to about 18,000 IU/mL, about 10,000 IU/mL to about 19,000 IU/mL, about 10,000 IU/mL to about 20,000 IU/mL, about 11,000 IU/mL to about 12,000 IU/mL, about 11,000 IU/mL to about 13,000 IU/mL, about 11,000 IU/mL to about 14,000 IU/mL, about 11,000 IU/mL to about 15,000 IU/mL, about 11,000 IU/mL to about 16,000 IU/mL, about 11,000 IU/mL to about 17,000 IU/mL, about 11,000 IU/mL to about 18,000 IU/mL, about 11,000 IU/mL to about 19,000 IU/mL, about 11,000 IU/mL to about 20,000 IU/mL, about 12,000 IU/mL to about 13,000 IU/mL, about 12,000 IU/mL to about 14,000 IU/mL, about 12,000 IU/mL to about 15,000 IU/mL, about 12,000 IU/mL to about 16,000 IU/mL, about 12,000 IU/mL to about 17,000 IU/mL, about 12,000 IU/mL to about 18,000 IU/mL, about 12,000 IU/mL to about 19,000 IU/mL, about 12,000 IU/mL to about 20,000 IU/mL, about 13,000 IU/mL to about 14,000 IU/mL, about 13,000 IU/mL to about 15,000 IU/mL, about 13,000 IU/mL to about 16,000 IU/mL, about 13,000 IU/mL to about 17,000 IU/mL, about 13,000 IU/mL to about 18,000 IU/mL, about 13,000 IU/mL to about 19,000 IU/mL, about 13,000 IU/mL to about 20,000 IU/mL, about 14,000 IU/mL to about 15,000 IU/mL, about 14,000 IU/mL to about 16,000 IU/mL, about 14,000 IU/mL to about 17,000 IU/mL, about 14,000 IU/mL to about 18,000 IU/mL, about 14,000 IU/mL to about 19,000 IU/mL, about 14,000 IU/mL to about 20,000 IU/mL, about 15,000 IU/mL to about 16,000 IU/mL, about 15,000 IU/mL to about 17,000 IU/mL, about 15,000 IU/mL to about 18,000 IU/mL, about 15,000 IU/mL to about 19,000 IU/mL, about 15,000 IU/mL to about 20,000 IU/mL, about 16,000 IU/mL to about 17,000 IU/mL, about 16,000 IU/mL to about 18,000 IU/mL, about 16,000 IU/mL to about 19,000 IU/mL, about 16,000 IU/mL to about 20,000 IU/mL, about 17,000 IU/mL to about 18,000 IU/mL, about 17,000 IU/mL to about 19,000 IU/mL, about 17,000 IU/mL to about 20,000 IU/mL, about 18,000 IU/mL to about 19,000 IU/mL, about 18,000 IU/mL to about 20,000 IU/mL, or about 19,000 IU/mL to about 20,000 IU/mL.
29. The use according to any one of embodiments 18-25, wherein the composition comprises the hyaluronidase in a concentration of at least 250 IU/mL, at least 500 IU/mL, at least 750 IU/mL, at least 1,000 IU/mL, at least 1,500 IU/mL, at least 2,000 IU/mL, at least 2,500 IU/mL, at least 3,000 IU/mL, at least 3,500 IU/mL, at least 4,000 IU/mL, at least 4,500 IU/mL, or at least 5,000 IU/mL; and/or a concentration of at most 250 IU/mL, at most 500 IU/mL, at most 750 IU/mL, at most 1,000 IU/mL, at most 1,500 IU/mL, at most 2,000 IU/mL, at most 2,500 IU/mL, at most 3,000 IU/mL, at most 3,500 IU/mL, at most 4,000 IU/mL, at most 4,500 IU/mL, or at most 5,000 IU/mL; or in a concentration of about 250 IU/mL to about 500 IU/mL, about 250 IU/mL to about 750 IU/mL, about 250 IU/mL to about 1,000 IU/mL, about 250 IU/mL to about 1,500 IU/mL, about 250 IU/mL to about 2,000 IU/mL, about 250 IU/mL to about 2,500 IU/mL, about 250 IU/mL to about 3,000 IU/mL, about 250 IU/mL to about 3,500 IU/mL, about 250 IU/mL to about 4,000 IU/mL, about 250 IU/mL to about 4,500 IU/mL, about 250 IU/mL to about 5,000 IU/mL, about 500 IU/mL to about 750 IU/mL, about 500 IU/mL to about 1,000 IU/mL, about 500 IU/mL to about 1,500 IU/mL, about 500 IU/mL to about 2,000 IU/mL, about 500 IU/mL to about 2,500 IU/mL, about 500 IU/mL to about 3,000 IU/mL, about 500 IU/mL to about 3,500 IU/mL, about 500 IU/mL to about 4,000 IU/mL, about 500 IU/mL to about 4,500 IU/mL, about 500 IU/mL to about 5,000 IU/mL, about 750 IU/mL to about 1,000 IU/mL, about 750 IU/mL to about 1,500 IU/mL, about 750 IU/mL to about 2,000 IU/mL, about 750 IU/mL to about 2,500 IU/mL, about 750 IU/mL to about 3,000 IU/mL, about 750 IU/mL to about 3,500 IU/mL, about 750 IU/mL to about 4,000 IU/mL, about 750 IU/mL to about 4,500 IU/mL, about 750 IU/mL to about 5,000 IU/mL, about 1,000 IU/mL to about 1,500 IU/mL, about 1,000 IU/mL to about 2,000 IU/mL, about 1,000 IU/mL to about 2,500 IU/mL, about 1,000 IU/mL to about 3,000 IU/mL, about 1,000 IU/mL to about 3,500 IU/mL, about 1,000 IU/mL to about 4,000 IU/mL, about 1,000 IU/mL to about 4,500 IU/mL, about 1,000 IU/mL to about 5,000 IU/mL, about 1,500 IU/mL to about 2,000 IU/mL, about 1,500 IU/mL to about 2,500 IU/mL, about 1,500 IU/mL to about 3,000 IU/mL, about 1,500 IU/mL to about 3,500 IU/mL, about 1,500 IU/mL to about 4,000 IU/mL, about 1,500 IU/mL to about 4,500 IU/mL, about 1,500 IU/mL to about 5,000 IU/mL, about 2,000 IU/mL to about 2,500 IU/mL, about 2,000 IU/mL to about 3,000 IU/mL, about 2,000 IU/mL to about 3,500 IU/mL, about 2,000 IU/mL to about 4,000 IU/mL, about 2,000 IU/mL to about 4,500 IU/mL, about 2,000 IU/mL to about 5,000 IU/mL, about 2,500 IU/mL to about 3,000 IU/mL, about 2,500 IU/mL to about 3,500 IU/mL, about 2,500 IU/mL to about 4,000 IU/mL, about 2,500 IU/mL to about 4,500 IU/mL, about 2,500 IU/mL to about 5,000 IU/mL, about 3,000 IU/mL to about 3,500 IU/mL, about 3,000 IU/mL to about 4,000 IU/mL, about 3,000 IU/mL to about 4,500 IU/mL, about 3,000 IU/mL to about 5,000 IU/mL, about 3,500 IU/mL to about 4,000 IU/mL, about 3,500 IU/mL to about 4,500 IU/mL, about 3,500 IU/mL to about 5,000 IU/mL, about 4,000 IU/mL to about 4,500 IU/mL, about 4,000 IU/mL to about 5,000 IU/mL, or about 4,500 IU/mL to about 5,000 IU/mL.
30. The use according to any one of embodiments 18-29, wherein the hyper-concentrated hyaluronidase is administered in an amount of at least 25,000 IU/mL, at least 50,000 IU/mL, at least 75,000 IU/mL, at least 100,000 IU/mL, at least 125,000 IU/mL, at least 150,000 IU/mL, at least 175,000 IU/mL, at least 200,000 IU/mL, at least 225,000 IU/mL, at least 250,000 IU/mL, at least 300,000 IU/mL, at least 350,000 IU/mL, at least 400,000 IU/mL, at least 450,000 IU/mL, or at least 500,000 IU/mL; and/or in an amount at most 25,000 IU/mL, at most 50,000 IU/mL, at most 75,000 IU/mL, at most 100,000 IU/mL, at most 125,000 IU/mL, at most 150,000 IU/mL, at most 175,000 IU/mL, at most 200,000 IU/mL, at most 225,000 IU/mL, at most 250,000 IU/mL, at most 300,000 IU/mL, at most 350,000 IU/mL, at most 400,000 IU/mL, at most 450,000 IU/mL, or at most 500,000 IU/mL; or in an amount of about 25,000 IU/mL to about 50,000 IU/mL, about 25,000 IU/mL to about 75,000 IU/mL, about 25,000 IU/mL to about 100,000 IU/mL, about 25,000 IU/mL to about 125,000 IU/mL, about 25,000 IU/mL to about 150,000 IU/mL, about 25,000 IU/mL to about 175,000 IU/mL, about 25,000 IU/mL to about 200,000 IU/mL, about 25,000 IU/mL to about 225,000 IU/mL, about 25,000 IU/mL to about 250,000 IU/mL, about 25,000 IU/mL to about 300,000 IU/mL, about 25,000 IU/mL to about 350,000 IU/mL, about 25,000 IU/mL to about 400,000 IU/mL, about 25,000 IU/mL to about 450,000 IU/mL, about 25,000 IU/mL to about 500,000 IU/mL, about 50,000 IU/mL to about 75,000 IU/mL, about 50,000 IU/mL to about 100,000 IU/mL, about 50,000 IU/mL to about 125,000 IU/mL, about 50,000 IU/mL to about 150,000 IU/mL, about 50,000 IU/mL to about 175,000 IU/mL, about 50,000 IU/mL to about 200,000 IU/mL, about 50,000 IU/mL to about 225,000 IU/mL, about 50,000 IU/mL to about 250,000 IU/mL, about 50,000 IU/mL to about 300,000 IU/mL, about 50,000 IU/mL to about 350,000 IU/mL, about 50,000 IU/mL to about 400,000 IU/mL, about 50,000 IU/mL to about 450,000 IU/mL, about 50,000 IU/mL to about 500,000 IU/mL, about 75,000 IU/mL to about 100,000 IU/mL, about 75,000 IU/mL to about 125,000 IU/mL, about 75,000 IU/mL to about 150,000 IU/mL, about 75,000 IU/mL to about 175,000 IU/mL, about 75,000 IU/mL to about 200,000 IU/mL, about 75,000 IU/mL to about 225,000 IU/mL, about 75,000 IU/mL to about 250,000 IU/mL, about 75,000 IU/mL to about 300,000 IU/mL, about 75,000 IU/mL to about 350,000 IU/mL, about 75,000 IU/mL to about 400,000 IU/mL, about 75,000 IU/mL to about 450,000 IU/mL, about 75,000 IU/mL to about 500,000 IU/mL, about 100,000 IU/mL to about 125,000 IU/mL, about 100,000 IU/mL to about 150,000 IU/mL, about 100,000 IU/mL to about 175,000 IU/mL, about 100,000 IU/mL to about 200,000 IU/mL, about 100,000 IU/mL to about 225,000 IU/mL, about 100,000 IU/mL to about 250,000 IU/mL, about 100,000 IU/mL to about 300,000 IU/mL, about 100,000 IU/mL to about 350,000 IU/mL, about 100,000 IU/mL to about 400,000 IU/mL, about 100,000 IU/mL to about 450,000 IU/mL, about 100,000 IU/mL to about 500,000 IU/mL, about 150,000 IU/mL to about 200,000 IU/mL, about 150,000 IU/mL to about 225,000 IU/mL, about 150,000 IU/mL to about 250,000 IU/mL, about 150,000 IU/mL to about 300,000 IU/mL, about 150,000 IU/mL to about 350,000 IU/mL, about 150,000 IU/mL to about 400,000 IU/mL, about 150,000 IU/mL to about 450,000 IU/mL, about 150,000 IU/mL to about 500,000 IU/mL, about 200,000 IU/mL to about 225,000 IU/mL, about 200,000 IU/mL to about 250,000 IU/mL, about 200,000 IU/mL to about 300,000 IU/mL, about 200,000 IU/mL to about 350,000 IU/mL, about 200,000 IU/mL to about 400,000 IU/mL, about 200,000 IU/mL to about 450,000 IU/mL, about 200,000 IU/mL to about 500,000 IU/mL, about 250,000 IU/mL to about 300,000 IU/mL, about 250,000 IU/mL to about 350,000 IU/mL, about 250,000 IU/mL to about 400,000 IU/mL, about 250,000 IU/mL to about 450,000 IU/mL, about 250,000 IU/mL to about 500,000 IU/mL, about 300,000 IU/mL to about 350,000 IU/mL, about 300,000 IU/mL to about 400,000 IU/mL, about 300,000 IU/mL to about 450,000 IU/mL, about 300,000 IU/mL to about 500,000 IU/mL, about 350,000 IU/mL to about 400,000 IU/mL, about 350,000 IU/mL to about 450,000 IU/mL, about 350,000 IU/mL to about 500,000 IU/mL, about 400,000 IU/mL to about 450,000 IU/mL, about 400,000 IU/mL to about 500,000 IU/mL, or about 450,000 IU/mL to about 500,000 IU/mL.
31. The use according to any one of embodiments 18-29, wherein the hyper-concentrated hyaluronidase is administered in an amount of at least 10,000 IU/mL, at least 20,000 IU/mL, at least 30,000 IU/mL, at least 40,000 IU/mL, at least 50,000 IU/mL, at least 60,000 IU/mL, at least 70,000 IU/mL, at least 80,000 IU/mL, at least 90,000 IU/mL, or at least 100,000 IU/mL; and/or in an amount at most 10,000 IU/mL, at most 20,000 IU/mL, at most 30,000 IU/mL, at most 40,000 IU/mL, at most 50,000 IU/mL, at most 60,000 IU/mL, at most 70,000 IU/mL, at most 80,000 IU/mL, at most 90,000 IU/mL, or at most 100,000 IU/mL; or in an amount of about 10,000 IU/mL to about 20,000 IU/mL, about 10,000 IU/mL to about 30,000 IU/mL, about 10,000 IU/mL to about 40,000 IU/mL, about 10,000 IU/mL to about 50,000 IU/mL, about 10,000 IU/mL to about 60,000 IU/mL, about 10,000 IU/mL to about 70,000 IU/mL, about 10,000 IU/mL to about 80,000 IU/mL, about 10,000 IU/mL to about 90,000 IU/mL, about 10,000 IU/mL to about 100,000 IU/mL, about 20,000 IU/mL to about 30,000 IU/mL, about 20,000 IU/mL to about 40,000 IU/mL, about 20,000 IU/mL to about 50,000 IU/mL, about 20,000 IU/mL to about 60,000 IU/mL, about 20,000 IU/mL to about 70,000 IU/mL, about 20,000 IU/mL to about 80,000 IU/mL, about 20,000 IU/mL to about 90,000 IU/mL, about 20,000 IU/mL to about 100,000 IU/mL, about 30,000 IU/mL to about 40,000 IU/mL, about 30,000 IU/mL to about 50,000 IU/mL, about 30,000 IU/mL to about 60,000 IU/mL, about 30,000 IU/mL to about 70,000 IU/mL, about 30,000 IU/mL to about 80,000 IU/mL, about 30,000 IU/mL to about 90,000 IU/mL, about 30,000 IU/mL to about 100,000 IU/mL, about 40,000 IU/mL to about 50,000 IU/mL, about 40,000 IU/mL to about 60,000 IU/mL, about 40,000 IU/mL to about 70,000 IU/mL, about 40,000 IU/mL to about 80,000 IU/mL, about 40,000 IU/mL to about 90,000 IU/mL, about 40,000 IU/mL to about 100,000 IU/mL, about 50,000 IU/mL to about 60,000 IU/mL, about 50,000 IU/mL to about 70,000 IU/mL, about 50,000 IU/mL to about 80,000 IU/mL, about 50,000 IU/mL to about 90,000 IU/mL, about 50,000 IU/mL to about 100,000 IU/mL, about 60,000 IU/mL to about 70,000 IU/mL, about 60,000 IU/mL to about 80,000 IU/mL, about 60,000 IU/mL to about 90,000 IU/mL, about 60,000 IU/mL to about 100,000 IU/mL, about 70,000 IU/mL to about 80,000 IU/mL, about 70,000 IU/mL to about 90,000 IU/mL, about 70,000 IU/mL to about 100,000 IU/mL, about 80,000 IU/mL to about 90,000 IU/mL, about 80,000 IU/mL to about 100,000 IU/mL, or about 90,000 IU/mL to about 100,000 IU/mL.
32. The use according to any one of embodiments 18-29, wherein the hyper-concentrated hyaluronidase is administered in an amount of at least 5,000 IU/mL, at least 6,000 IU/mL, at least 7,000 IU/mL, at least 8,000 IU/mL, at least 9,000 IU/mL, at least 10,000 IU/mL, at least 11,000 IU/mL, at least 12,000 IU/mL, at least 13,000 IU/mL, at least 14,000 IU/mL, at least 15,000 IU/mL, at least 16,000 IU/mL, at least 17,000 IU/mL, at least 18,000 IU/mL, at least 19,000 IU/mL, or at least 20,000 IU/mL; and/or in an amount at most 5,000 IU/mL, at most 6,000 IU/mL, at most 7,000 IU/mL, at most 8,000 IU/mL, at most 9,000 IU/mL, at most 10,000 IU/mL, at most 11,000 IU/mL, at most 12,000 IU/mL, at most 13,000 IU/mL, at most 14,000 IU/mL, at most 15,000 IU/mL, at most 16,000 IU/mL, at most 17,000 IU/mL, at most 18,000 IU/mL, at most 19,000 IU/mL, or at most 20,000 IU/mL; or in an amount of about 5,000 IU/mL to about 6,000 IU/mL, about 5,000 IU/mL to about 7,000 IU/mL, about 5,000 IU/mL to about 8,000 IU/mL, about 5,000 IU/mL to about 9,000 IU/mL, about 5,000 IU/mL to about 10,000 IU/mL, about 5,000 IU/mL to about 11,000 IU/mL, about 5,000 IU/mL to about 12,000 IU/mL, about 5,000 IU/mL to about 13,000 IU/mL, about 5,000 IU/mL to about 14,000 IU/mL, about 5,000 IU/mL to about 15,000 IU/mL, about 5,000 IU/mL to about 16,000 IU/mL, about 5,000 IU/mL to about 17,000 IU/mL, about 5,000 IU/mL to about 18,000 IU/mL, about 5,000 IU/mL to about 19,000 IU/mL, about 5,000 IU/mL to about 20,000 IU/mL, about 6,000 IU/mL to about 7,000 IU/mL, about 6,000 IU/mL to about 8,000 IU/mL, about 6,000 IU/mL to about 9,000 IU/mL, about 6,000 IU/mL to about 10,000 IU/mL, about 6,000 IU/mL to about 11,000 IU/mL, about 6,000 IU/mL to about 12,000 IU/mL, about 6,000 IU/mL to about 13,000 IU/mL, about 6,000 IU/mL to about 14,000 IU/mL, about 6,000 IU/mL to about 15,000 IU/mL, about 6,000 IU/mL to about 16,000 IU/mL, about 6,000 IU/mL to about 17,000 IU/mL, about 6,000 IU/mL to about 18,000 IU/mL, about 6,000 IU/mL to about 19,000 IU/mL, about 6,000 IU/mL to about 20,000 IU/mL, about 7,000 IU/mL to about 8,000 IU/mL, about 7,000 IU/mL to about 9,000 IU/mL, about 7,000 IU/mL to about 10,000 IU/mL, about 7,000 IU/mL to about 11,000 IU/mL, about 7,000 IU/mL to about 12,000 IU/mL, about 7,000 IU/mL to about 13,000 IU/mL, about 7,000 IU/mL to about 14,000 IU/mL, about 7,000 IU/mL to about 15,000 IU/mL, about 7,000 IU/mL to about 16,000 IU/mL, about 7,000 IU/mL to about 17,000 IU/mL, about 7,000 IU/mL to about 18,000 IU/mL, about 7,000 IU/mL to about 19,000 IU/mL, about 7,000 IU/mL to about 20,000 IU/mL, about 8,000 IU/mL to about 9,000 IU/mL, about 8,000 IU/mL to about 10,000 IU/mL, about 8,000 IU/mL to about 11,000 IU/mL, about 8,000 IU/mL to about 12,000 IU/mL, about 8,000 IU/mL to about 13,000 IU/mL, about 8,000 IU/mL to about 14,000 IU/mL, about 8,000 IU/mL to about 15,000 IU/mL, about 8,000 IU/mL to about 16,000 IU/mL, about 8,000 IU/mL to about 17,000 IU/mL, about 8,000 IU/mL to about 18,000 IU/mL, about 8,000 IU/mL to about 19,000 IU/mL, about 8,000 IU/mL to about 20,000 IU/mL, about 9,000 IU/mL to about 10,000 IU/mL, about 9,000 IU/mL to about 11,000 IU/mL, about 9,000 IU/mL to about 12,000 IU/mL, about 9,000 IU/mL to about 13,000 IU/mL, about 9,000 IU/mL to about 14,000 IU/mL, about 9,000 IU/mL to about 15,000 IU/mL, about 9,000 IU/mL to about 16,000 IU/mL, about 9,000 IU/mL to about 17,000 IU/mL, about 9,000 IU/mL to about 18,000 IU/mL, about 9,000 IU/mL to about 19,000 IU/mL, about 9,000 IU/mL to about 20,000 IU/mL, about 10,000 IU/mL to about 11,000 IU/mL, about 10,000 IU/mL to about 12,000 IU/mL, about 10,000 IU/mL to about 13,000 IU/mL, about 10,000 IU/mL to about 14,000 IU/mL, about 10,000 IU/mL to about 15,000 IU/mL, about 10,000 IU/mL to about 16,000 IU/mL, about 10,000 IU/mL to about 17,000 IU/mL, about 10,000 IU/mL to about 18,000 IU/mL, about 10,000 IU/mL to about 19,000 IU/mL, about 10,000 IU/mL to about 20,000 IU/mL, about 11,000 IU/mL to about 12,000 IU/mL, about 11,000 IU/mL to about 13,000 IU/mL, about 11,000 IU/mL to about 14,000 IU/mL, about 11,000 IU/mL to about 15,000 IU/mL, about 11,000 IU/mL to about 16,000 IU/mL, about 11,000 IU/mL to about 17,000 IU/mL, about 11,000 IU/mL to about 18,000 IU/mL, about 11,000 IU/mL to about 19,000 IU/mL, about 11,000 IU/mL to about 20,000 IU/mL, about 12,000 IU/mL to about 13,000 IU/mL, about 12,000 IU/mL to about 14,000 IU/mL, about 12,000 IU/mL to about 15,000 IU/mL, about 12,000 IU/mL to about 16,000 IU/mL, about 12,000 IU/mL to about 17,000 IU/mL, about 12,000 IU/mL to about 18,000 IU/mL, about 12,000 IU/mL to about 19,000 IU/mL, about 12,000 IU/mL to about 20,000 IU/mL, about 13,000 IU/mL to about 14,000 IU/mL, about 13,000 IU/mL to about 15,000 IU/mL, about 13,000 IU/mL to about 16,000 IU/mL, about 13,000 IU/mL to about 17,000 IU/mL, about 13,000 IU/mL to about 18,000 IU/mL, about 13,000 IU/mL to about 19,000 IU/mL, about 13,000 IU/mL to about 20,000 IU/mL, about 14,000 IU/mL to about 15,000 IU/mL, about 14,000 IU/mL to about 16,000 IU/mL, about 14,000 IU/mL to about 17,000 IU/mL, about 14,000 IU/mL to about 18,000 IU/mL, about 14,000 IU/mL to about 19,000 IU/mL, about 14,000 IU/mL to about 20,000 IU/mL, about 15,000 IU/mL to about 16,000 IU/mL, about 15,000 IU/mL to about 17,000 IU/mL, about 15,000 IU/mL to about 18,000 IU/mL, about 15,000 IU/mL to about 19,000 IU/mL, about 15,000 IU/mL to about 20,000 IU/mL, about 16,000 IU/mL to about 17,000 IU/mL, about 16,000 IU/mL to about 18,000 IU/mL, about 16,000 IU/mL to about 19,000 IU/mL, about 16,000 IU/mL to about 20,000 IU/mL, about 17,000 IU/mL to about 18,000 IU/mL, about 17,000 IU/mL to about 19,000 IU/mL, about 17,000 IU/mL to about 20,000 IU/mL, about 18,000 IU/mL to about 19,000 IU/mL, about 18,000 IU/mL to about 20,000 IU/mL, or about 19,000 IU/mL to about 20,000 IU/mL.
33. The use according to any one of embodiments 18-29, wherein the hyper-concentrated hyaluronidase is administered in an amount of at least 250 IU/mL, at least 500 IU/mL, at least 750 IU/mL, at least 1,000 IU/mL, at least 1,500 IU/mL, at least 2,000 IU/mL, at least 2,500 IU/mL, at least 3,000 IU/mL, at least 3,500 IU/mL, at least 4,000 IU/mL, at least 4,500 IU/mL, or at least 5,000 IU/mL; and/or an amount of at most 250 IU/mL, at most 500 IU/mL, at most 750 IU/mL, at most 1,000 IU/mL, at most 1,500 IU/mL, at most 2,000 IU/mL, at most 2,500 IU/mL, at most 3,000 IU/mL, at most 3,500 IU/mL, at most 4,000 IU/mL, at most 4,500 IU/mL, or at most 5,000 IU/mL; or in an amount of about 250 IU/mL to about 500 IU/mL, about 250 IU/mL to about 750 IU/mL, about 250 IU/mL to about 1,000 IU/mL, about 250 IU/mL to about 1,500 IU/mL, about 250 IU/mL to about 2,000 IU/mL, about 250 IU/mL to about 2,500 IU/mL, about 250 IU/mL to about 3,000 IU/mL, about 250 IU/mL to about 3,500 IU/mL, about 250 IU/mL to about 4,000 IU/mL, about 250 IU/mL to about 4,500 IU/mL, about 250 IU/mL to about 5,000 IU/mL, about 500 IU/mL to about 750 IU/mL, about 500 IU/mL to about 1,000 IU/mL, about 500 IU/mL to about 1,500 IU/mL, about 500 IU/mL to about 2,000 IU/mL, about 500 IU/mL to about 2,500 IU/mL, about 500 IU/mL to about 3,000 IU/mL, about 500 IU/mL to about 3,500 IU/mL, about 500 IU/mL to about 4,000 IU/mL, about 500 IU/mL to about 4,500 IU/mL, about 500 IU/mL to about 5,000 IU/mL, about 750 IU/mL to about 1,000 IU/mL, about 750 IU/mL to about 1,500 IU/mL, about 750 IU/mL to about 2,000 IU/mL, about 750 IU/mL to about 2,500 IU/mL, about 750 IU/mL to about 3,000 IU/mL, about 750 IU/mL to about 3,500 IU/mL, about 750 IU/mL to about 4,000 IU/mL, about 750 IU/mL to about 4,500 IU/mL, about 750 IU/mL to about 5,000 IU/mL, about 1,000 IU/mL to about 1,500 IU/mL, about 1,000 IU/mL to about 2,000 IU/mL, about 1,000 IU/mL to about 2,500 IU/mL, about 1,000 IU/mL to about 3,000 IU/mL, about 1,000 IU/mL to about 3,500 IU/mL, about 1,000 IU/mL to about 4,000 IU/mL, about 1,000 IU/mL to about 4,500 IU/mL, about 1,000 IU/mL to about 5,000 IU/mL, about 1,500 IU/mL to about 2,000 IU/mL, about 1,500 IU/mL to about 2,500 IU/mL, about 1,500 IU/mL to about 3,000 IU/mL, about 1,500 IU/mL to about 3,500 IU/mL, about 1,500 IU/mL to about 4,000 IU/mL, about 1,500 IU/mL to about 4,500 IU/mL, about 1,500 IU/mL to about 5,000 IU/mL, about 2,000 IU/mL to about 2,500 IU/mL, about 2,000 IU/mL to about 3,000 IU/mL, about 2,000 IU/mL to about 3,500 IU/mL, about 2,000 IU/mL to about 4,000 IU/mL, about 2,000 IU/mL to about 4,500 IU/mL, about 2,000 IU/mL to about 5,000 IU/mL, about 2,500 IU/mL to about 3,000 IU/mL, about 2,500 IU/mL to about 3,500 IU/mL, about 2,500 IU/mL to about 4,000 IU/mL, about 2,500 IU/mL to about 4,500 IU/mL, about 2,500 IU/mL to about 5,000 IU/mL, about 3,000 IU/mL to about 3,500 IU/mL, about 3,000 IU/mL to about 4,000 IU/mL, about 3,000 IU/mL to about 4,500 IU/mL, about 3,000 IU/mL to about 5,000 IU/mL, about 3,500 IU/mL to about 4,000 IU/mL, about 3,500 IU/mL to about 4,500 IU/mL, about 3,500 IU/mL to about 5,000 IU/mL, about 4,000 IU/mL to about 4,500 IU/mL, about 4,000 IU/mL to about 5,000 IU/mL, or about 4,500 IU/mL to about 5,000 IU/mL.
34. The method according to any one of embodiments 18-33, wherein the composition or medicament is administered in a single dose or in multiple doses.
35. The use according to any one of embodiments 18-34, wherein the composition or medicament is administered in a volume of at least 0.5 mL, at least 1.0 mL, at least 2.0 mL, at least 3.0 mL, at least 4.0 mL, or at least 5.0 mL; and/or in a volume of at most 0.5 mL, at most 1.0 mL, at most 2.0 mL, at most 3.0 mL, at most 4.0 mL, or at most 5.0 mL; or in a volume of about 0.5 mL to 1.0 mL, about 0.5 mL to 2.0 mL, about 0.5 mL to 3.0 mL, about 0.5 mL to 4.0 mL, about 0.5 mL to 5.0 mL, about 1.0 mL to 2.0 mL, about 1.0 mL to 3.0 mL, about 1.0 mL to 4.0 mL, about 1.0 to 5.0 mL, about 2.0 mL to 3.0 mL, about 2.0 mL to 4.0 mL, about 2.0 to 5.0 mL, about 3.0 mL to 4.0 mL, about 3.0 to 5.0 mL, or about 4.0 to 5.0 mL.
36. The use according to any one of embodiments 26-35, wherein the composition or medicament enters an area of tissue in at most 1 minute, at most 2 minutes, at most 3 minutes, at most 4 minutes, at most 5 minutes, at most 10 minutes, at most 15 minutes, at most 30 minutes, at most 45 minutes, at most 60 minutes, at most 75 minutes or at most 90 minutes; or in about 1 minute to about 2 minutes, about 1 minute to about 3 minutes, about 1 minute to about 5 minutes, about 1 minute to about 7 minutes, about 1 minute to about 10 minutes, about 1 minute to about 15 minutes, about 1 minute to about 30 minutes, about 1 minute to about 45 minutes, about 1 minute to about 60 minutes, about 1 minute to about 75 minutes, about 1 minute to about 90 minutes, about 3 minutes to about 5 minutes, about 3 minutes to about 7 minutes, about 3 minutes to about 10 minutes, about 3 minutes to about 15 minutes, about 3 minutes to about 30 minutes, about 3 minutes to about 45 minutes, about 3 minutes to about 60 minutes, about 3 minutes to about 75 minutes, about 3 minutes to about 90 minutes, about 5 minutes to about 7 minutes, about 5 minutes to about 10 minutes, about 5 minutes to about 15 minutes, about 5 minutes to about 30 minutes, about 5 minutes to about 45 minutes, about 5 minutes to about 60 minutes, about 5 minutes to about 75 minutes, about 5 minutes to about 90 minutes, about 10 minutes to about 15 minutes, about 10 minutes to about 30 minutes, about 10 minutes to about 45 minutes, about 10 minutes to about 60 minutes, about 10 minutes to about 75 minutes, about 10 minutes to about 90 minutes, about 15 minutes to about 30 minutes, about 15 minutes to about 45 minutes, about 15 minutes to about 60 minutes, about 15 minutes to about 75 minutes, about 15 minutes to about 90 minutes.
37. The use according to any one of embodiments 18-36, wherein the hyaluronic acid-induced blockage in the one or more blood vessels supplying blood to the area of tissue being affected by the blockage is reduced or eliminated in at most 1 minute, at most 2 minutes, at most 3 minutes, at most 4 minutes, at most 5 minutes, at most 10 minutes, at most 15 minutes, at most 30 minutes, at most 45 minutes, at most 60 minutes, at most 75 minutes or at most 90 minutes; or in about 1 minute to about 2 minutes, about 1 minute to about 3 minutes, about 1 minute to about 5 minutes, about 1 minute to about 7 minutes, about 1 minute to about 10 minutes, about 1 minute to about 15 minutes, about 1 minute to about 30 minutes, about 1 minute to about 45 minutes, about 1 minute to about 60 minutes, about 1 minute to about 75 minutes, about 1 minute to about 90 minutes, about 3 minutes to about 5 minutes, about 3 minutes to about 7 minutes, about 3 minutes to about 10 minutes, about 3 minutes to about 15 minutes, about 3 minutes to about 30 minutes, about 3 minutes to about 45 minutes, about 3 minutes to about 60 minutes, about 3 minutes to about 75 minutes, about 3 minutes to about 90 minutes, about 5 minutes to about 7 minutes, about 5 minutes to about 10 minutes, about 5 minutes to about 15 minutes, about 5 minutes to about 30 minutes, about 5 minutes to about 45 minutes, about 5 minutes to about 60 minutes, about 5 minutes to about 75 minutes, about 5 minutes to about 90 minutes, about 10 minutes to about 15 minutes, about 10 minutes to about 30 minutes, about 10 minutes to about 45 minutes, about 10 minutes to about 60 minutes, about 10 minutes to about 75 minutes, about 10 minutes to about 90 minutes, about 15 minutes to about 30 minutes, about 15 minutes to about 45 minutes, about 15 minutes to about 60 minutes, about 15 minutes to about 75 minutes, about 15 minutes to about 90 minutes.
38. A kit comprising one or more containers, with each container including a pharmaceutical composition including a hyper-concentrated hyaluronidase.
39. The kit according to embodiment 38, wherein the pharmaceutical composition is in a liquid formulation.
40. The kit according to embodiment 38, wherein the pharmaceutical composition is in a dried formulation.
41. The kit according to any one of embodiments 38-40, wherein the one or more containers are each a delivery system.
42. The kit according to any one of embodiments 38-40, further comprising a delivery system.
43. The kit according to any one of embodiments 38-42, further comprising another container including a solvent.
44. The kit according to any one of embodiments 38-43, further comprising instructions.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, pharmaceutical compositions, or methods and uses disclosed herein.

### Example 1

### Rabbit Model of Extravascular HA Dermal Filler Removal Using Hyaluronidase

This example illustrates how to assess the safety and efficacy of a hyaluronidase treatment disclosed herein using an extravascular dermal filler animal model. New Zealand white rabbits will be used in this study. These animals will be selected due to the similarity of their vascular anatomy to that of humans. New Zealand white rabbits weighing 2.0 to 4.5 kg will be divided into groups based on the hyaluronidase dose and administration time.

In one series of experiments, animals were divided into three groups with each animal being subcutaneously injected with 0.2 mL of a dermal filler containing lidocaine at three different sites along the back of the animal. The dermal filler at each injection site for each animal was then evaluated using ultrasound sonography imaging. After this baseline analysis was completed, animals were then subjected to one of three treatments. Group 1 animals (Control Animals) were treated by injecting 0.2 mL of Phosphate Buffered Saline (PBS) at the dermal filler injection sites. Group 2 animals were treated by injecting 0.2 mL of a low-concentration hyaluronidase (150 IU/mL) at the dermal filler injection sites. Group 3 animals were treated by injecting 0.2 mL of a high-concentration hyaluronidase (31,000 IU/mL) at the dermal filler injection sites. The presence of dermal filler at each injection site of all treated animals was then evaluated using ultrasound sonography imaging at 5 minutes, 10 minutes, 30 minutes, 60 minutes and 24 hours post-treatment. Ultrasound measurements of dermal filler at each injection site were measured for central area and 3D volume, and representative images of each dermal filler site were taken. The pre-treatment ultrasound measurement was taken as the baseline size and all other measurements were evaluated as a percent change from the pre-treatment ultrasound measurement.

As shown in Table 1, Group 1 animals, which received a PBS administration, did not exhibit any significant loss of dermal filler throughout the time course of the experiment. Interestingly, Group 2 animals, which received a low-concentration hyaluronidase (150 IU/mL), also failed to show any significant loss of dermal filler. On the other hand, Group 3 animals, which received a high-concentration hyaluronidase (40,000 IU/mL), exhibited significant loss of dermal filler, with an about 20% size loss at the 30-minute post-treatment timepoint, an about 35% size loss at the 45-minute post-treatment timepoint, and a complete loss of dermal filler at the 24-hour post-treatment timepoint. The increase in percent sizes of dermal filler in each group may be secondary to tissue swelling associated with the injection of PBS or hyaluronidase, as the case may be.

TABLE 1
Analysis of Extravascular Injected Dermal Filler

| | Percent Size Dermal Filler (%) | | |
|---|---|---|---|
| | Group 1 | Group 2 | Group 3 |
| Time | (n = 1) | (n = 1) | (n = 1) |
| Pre-Treatment Baseline | 100 | 100 | 100 |
| 0 min Post-Treatment | 102.4 | 100 | 118.9 |
| 5 min Post-Treatment | 92.6 | 97.2 | 128.8 |
| 10 min Post-Treatment | 103.3 | 107.2 | 112.5 |
| 15 min Post-Treatment | 105.2 | 106.1 | 102.8 |
| 30 min Post-Treatment | ND | 99.4 | 79.5 |
| 45 min Post-Treatment | ND | 111.2 | 64.8 |
| 24 hours Post-Treatment | 106.3 | 114.5 | 0 |

ND, Not determined.

### Example 2

### Rabbit Model of Intravascular HA Dermal Filler Removal Using Hyaluronidase

This example illustrates how to assess the safety and efficacy of a hyaluronidase treatment disclosed herein using an intravascular dermal filler animal model. New Zealand white rabbits will be used in this study. These animals will be selected due to the similarity of their vascular anatomy to that of humans. New Zealand white rabbits weighing 2.0 to 4.5 kg will be divided into groups based on the hyaluronidase dose and administration time.

In one series of experiments, animals were divided into three groups with each animal being injected into the left and right auricular artery with 0.1 mL of a dermal filler containing lidocaine at one site for each ear of each animal. The dermal filler at each injection site for each animal was then evaluated using ultrasound sonography imaging. After this baseline analysis was completed, animals were then subjected to one of three treatments. Group 1 animals (Control Animals) were treated by intra-auricularly injecting 10 μL of PBS at each of the four sites containing a dermal filer occlusion. Group 2 animals were treated by intra-auricularly injecting 0.1 mL of a low-concentration hyaluronidase (150 IU/mL) at each of the four sites containing a dermal filer occlusion. Group 3 animals were treated by intra-auricularly injecting 10 μL of a high-concentration hyaluronidase (40,000 IU/mL) at each of the four sites containing a dermal filer occlusion. The presence of dermal filler at each injection site of all treated animals was then evaluated using ultrasound sonography imaging at 5 minutes, 10 minutes, 30 minutes, 60 minutes and 24 hours post-treatment. Ultrasound measurements of dermal filler at each injection site was measured for central area and 3D volume, and representative images of each dermal filler site was taken. The pre-treatment ultrasound measurement was taken as the baseline size and all other measurements were evaluated as a percent change from the pre-treatment ultrasound measurement.

As shown in Table 2, Group 1 animals, which received a PBS administration, did not exhibit any significant loss of dermal filler throughout the time course of the experiment. Group 2 animals, which received a low-concentration hyaluronidase (150 IU/mL), did show significant loss of dermal filler that was consistent throughout the time course, exhibiting an about 43% size loss at the 15-minute post-treatment timepoint, an about 50% size loss at the 30-minute post-treatment timepoint, an about 56% size loss at the 45-minute post-treatment timepoint, and a complete loss of dermal filler at the 24-hour post-treatment timepoint. Group 3 animals, which received a high-concentration hyaluronidase (31,000 IU/mL), also exhibited significant loss of dermal filler and at a dramatically increased rate of loss compared to Group 2 animals. For example, Group 3 animals exhibiting an about 70% size loss at the 15-minute post-treatment timepoint, an about 90% size loss at the 30-minute post-treatment timepoint, and a complete loss of dermal filler at the 45-minute post-treatment timepoint.

TABLE 2
Analysis of Intravascular Injected Dermal Filler

| | Percent Size Dermal Filler (%) | | |
|---|---|---|---|
| | Group 1 | Group 2 | Group 3 |
| Time | (n = 2) | (n = 4) | (n = 3) |
| Pre-Treatment Baseline | 100 | 100 | 100 |
| 0 min Post-Treatment | 106.9 | 80.6 | 68.2 |
| 5 min Post-Treatment | 100.9 | 64.7 | 41.1 |
| 10 min Post-Treatment | 105.3 | 60.5 | 33.1 |
| 15 min Post-Treatment | 101.4 | 57.4 | 29.6 |
| 30 min Post-Treatment | 104.8 | 49.7 | 10.5 |
| 45 min Post-Treatment | 107.1 | 43.5 | 0 |
| 24 hours Post-Treatment | 120.5 | 0 | 0 |

### Example 3

### Rabbit Model of Filler-Induced Vascular Occlusion

This example illustrates how to assess the safety and efficacy of a hyaluronidase treatment disclosed herein using an animal model for a filler-induced vascular occlusion event. New Zealand white rabbits will be used to simulate hyaluronic acid associated vascular occlusion(s) of an area of tissue. These animals will be selected due to the similarity of their vascular anatomy to that of humans. New Zealand white rabbits weighing 2.0 to 3.0 kg will be divided into groups based on the hyaluronidase dose and administration time.

Hyaluronic acid filler will be administered into the facial artery or a femoral artery of animals to create an arterial occlusion. This arterial occlusion as well as subsequent ischemia will be confirmed by and appropriate technique, such as, e.g., ultrasonography, angiography, nerve conduction study (NCS) assays, electromyography (EMG), and/or electroencephalogram (EEG) analyses. In addition, for femoral artery administration, a hind-paw assay will be performed. Different doses of hyper-concentrated hyaluronidase will be administered at several post-obstruction time points to assess both dosing and timing effectiveness of the treatment. Control animals will be injected with hyaluronic acid filler in the same manner as experimental animals but will receive no hyaluronidase treatment. Angiography and EEG changes will be recorded at 30, 60, 90, and 120 minutes after administration of hyaluronidase. EEG will be performed after 60 and 120 minutes to confirm the tissue reperfusion and electrophysiologic function.

In one series of experiments, an arterial occlusion will be induced by injecting 0.5 mL to 0.9 mL of hyaluronic acid filler into the left facial or left femoral artery of all rabbits, with the right facial or right femoral artery respectively serving as an untreated control. The animals will then be divided into six experimental groups each containing 2 or 3 animals with Group 1 animals each receiving 500 μL of a composition comprising a hyper-concentrated hyaluronidase (10,000 IU/mL) subdermally administered around the occlusion 5 minutes after the hyaluronic acid filler injection; Group 2 animals each receiving 500 μL of a composition comprising a hyper-concentrated hyaluronidase (10,000 IU/mL) subdermally administered around the occlusion 10 minutes after the hyaluronic acid filler injection; Group 3 animals each receiving 500 μL of a composition comprising a hyper-concentrated hyaluronidase (12,000 IU/mL) subdermally administered around the occlusion 5 minutes after the hyaluronic acid filler injection; Group 4 animals each receiving 500 μL of a composition comprising a hyper-concentrated hyaluronidase (12,000 IU/mL) subdermally administered around the occlusion 10 minutes after the hyaluronic acid filler injection; Group 5 animals each receiving 500 μL of a composition comprising a low-concentration hyaluronidase (150 IU/mL) subdermally administered around the occlusion 5 minutes after the hyaluronic acid filler injection; and Group 6 animals each receiving 500 μL of a composition comprising a low-concentration hyaluronidase (150 IU/mL) subdermally administered around the occlusion 10 minutes after the hyaluronic acid filler injection.

In another series of experiments, an arterial occlusion will be induced by injecting 0.5 mL to 0.9 mL of hyaluronic acid filler into the left facial or the left femoral artery of all rabbits, with the right facial or right femoral artery respectively serving as an untreated control. The animals will then be divided into six experimental groups each containing 2 or 3 animals with Group 1 animals each receiving 500 μL of a composition comprising a hyper-concentrated hyaluronidase (15,000 IU/mL) subdermally administered around the occlusion 5 minutes after the hyaluronic acid filler injection; Group 2 animals each receiving 500 μL of a composition comprising a hyper-concentrated hyaluronidase (15,000 IU/mL) subdermally administered around the occlusion 10 minutes after the hyaluronic acid filler injection; Group 3 animals each receiving 500 μL of a composition comprising a hyper-concentrated hyaluronidase (20,000 IU/mL) subdermally administered around the occlusion 5 minutes after hyaluronic acid filler injection; Group 4 animals each receiving 500 μL of a composition comprising a hyper-concentrated hyaluronidase (20,000 IU/mL) subdermally administered around the occlusion 10 minutes after the hyaluronic acid filler injection; Group 5 animals each receiving 500 μL of a composition comprising a low-concentration hyaluronidase (150 IU/mL) subdermally administered around the occlusion 5 minutes after the hyaluronic acid filler injection; and Group 6 animals each receiving 500 μL of a composition comprising a low-concentration hyaluronidase (150 IU/mL) subdermally administered around the occlusion 10 minutes after the hyaluronic acid filler injection.

In another series of experiments, an arterial occlusion will be induced by injecting 0.5 mL to 0.9 mL of hyaluronic acid filler into the left facial or the left femoral artery of all rabbits, with the right facial or right femoral artery respectively serving as an untreated control. The animals will then be divided into six experimental groups each containing 2 or 3 animals with Group 1 animals each receiving 500 μL of a composition comprising a hyper-concentrated hyaluronidase (10,000 IU/mL) subdermally administered around the occlusion 15 minutes after the hyaluronic acid filler injection; Group 2 animals each receiving 500 μL of a composition comprising a hyper-concentrated hyaluronidase (10,000 IU/mL) subdermally administered around the occlusion 30 minutes after the hyaluronic acid filler injection; Group 3 animals each receiving 500 μL of a composition comprising a hyper-concentrated hyaluronidase (12,000 IU/mL) subdermally administered around the occlusion 15 minutes after the hyaluronic acid filler injection; Group 4 animals each receiving 500 μL of a composition comprising a hyper-concentrated hyaluronidase (12,000 IU/mL) subdermally administered around the occlusion 30 minutes after the hyaluronic acid filler injection; Group 5 animals each receiving 500 μL of a composition comprising a low-concentration hyaluronidase (150 IU/mL) subdermally administered around the occlusion 5 minutes after the hyaluronic acid filler injection; and Group 6 animals each receiving 500 μL of a composition comprising a low-concentration hyaluronidase (150 IU/mL) subdermally administered around the occlusion 10 minutes after the hyaluronic acid filler injection.

In another series of experiments, an arterial occlusion will be induced by injecting 0.5 mL to 0.9 mL of hyaluronic acid filler into the left facial or the left femoral artery of all rabbits, with the right facial or right femoral artery respectively serving as an untreated control. The animals will then be divided into six experimental groups each containing 2 or 3 animals with Group 1 animals each receiving 500 μL of a composition comprising a hyper-concentrated hyaluronidase (15,000 IU/mL) subdermally administered around the occlusion 15 minutes after the hyaluronic acid filler injection; Group 2 animals each receiving 500 μL of a composition comprising a hyper-concentrated hyaluronidase (15,000 IU/mL) subdermally administered around the occlusion 30 minutes after the hyaluronic acid filler injection; Group 3 animals each receiving 500 μL of a composition comprising a hyper-concentrated hyaluronidase (20,000 IU/mL) subdermally administered around the occlusion 15 minutes after the hyaluronic acid filler injection; Group 4 animals each receiving 500 μL of a composition comprising a hyper-concentrated hyaluronidase (20,000 IU/mL) subdermally administered around the occlusion 30 minutes after the hyaluronic acid filler injection; Group 5 animals each receiving 500 μL of a composition comprising a low-concentration hyaluronidase (150 IU/mL) subdermally administered around the occlusion 5 minutes after the hyaluronic acid filler injection; and Group 6 animals each receiving 500 μL of a composition comprising a low-concentration hyaluronidase (150 IU/mL) subdermally administered around the occlusion 10 minutes after the hyaluronic acid filler injection.

In another series of experiments, an arterial occlusion will be induced by injecting 0.5 mL to 0.9 mL of hyaluronic acid filler into the left facial or the left femoral artery of all rabbits, with the right facial or right femoral artery respectively serving as an untreated control. The animals will then be divided into six experimental groups each containing 2 or 3 animals with Group 1 animals each receiving 500 μL of a composition comprising a hyper-concentrated hyaluronidase (10,000 IU/mL) subdermally administered around the occlusion 60 minutes after the hyaluronic acid filler injection; Group 2 animals each receiving 500 μL of a composition comprising a hyper-concentrated hyaluronidase (10,000 IU/mL) subdermally administered around the occlusion 120 minutes after the hyaluronic acid filler injection; Group 3 animals each receiving 500 μL of a composition comprising a hyper-concentrated hyaluronidase (12,000 IU/mL) subdermally administered around the occlusion 60 minutes after the hyaluronic acid filler injection; Group 4 animals each receiving 500 μL of a composition comprising a hyper-concentrated hyaluronidase (12,000 IU/mL) subdermally administered around the occlusion 120 minutes after the hyaluronic acid filler injection; Group 5 animals each receiving 500 μL of a composition comprising a low-concentration hyaluronidase (150 IU/mL) subdermally administered around the occlusion 5 minutes after the hyaluronic acid filler injection; and Group 6 animals each receiving 500 μL of a composition comprising a low-concentration hyaluronidase (150 IU/mL) subdermally administered around the occlusion 10 minutes after the hyaluronic acid filler injection.

In another series of experiments, an arterial occlusion will be induced by injecting 0.5 mL to 0.9 mL of hyaluronic acid filler into the left facial or the left femoral artery of all rabbits, with the right facial or right femoral artery respectively serving as an untreated control. The animals will then be divided into six experimental groups each containing 2 or 3 animals with Group 1 animals each receiving 500 μL of a composition comprising a hyper-concentrated hyaluronidase (15,000 IU/mL) subdermally administered around the occlusion 60 minutes after the hyaluronic acid filler injection; Group 2 animals each receiving 500 μL of a composition comprising a hyper-concentrated hyaluronidase (15,000 IU/mL) subdermally administered around the occlusion 120 minutes after the hyaluronic acid filler injection; Group 3 animals each receiving 500 μL of a composition comprising a hyper-concentrated hyaluronidase (20,000 IU/mL) subdermally administered around the occlusion 60 minutes after the hyaluronic acid filler injection; Group 4 animals each receiving 500 μL of a composition comprising a hyper-concentrated hyaluronidase (20,000 IU/mL) subdermally administered around the occlusion 120 minutes after the hyaluronic acid filler injection; Group 5 animals each receiving 500 μL of a composition comprising a low-concentration hyaluronidase (150 IU/mL) subdermally administered around the occlusion 5 minutes after the hyaluronic acid filler injection; and Group 6 animals each receiving 500 μL of a composition comprising a low-concentration hyaluronidase (150 IU/mL) subdermally administered around the occlusion 10 minutes after the hyaluronic acid filler injection.

Analysis of experimental animals by ultrasonography, angiography, NCS assays, EMG, and/or EEG analysis, as well as the hind paw assay, are expected to reveal no significant arterial occlusion, tissue damage, or loss of tissue after hyper-concentrated hyaluronidase treatment. Control animals, on the other hand, are expected to exhibit no improvement in filler-induced arterial occlusion and signs of tissue damage.

### Example 4

### Rabbit Model of Filler-Induced Vascular Occlusion

This example illustrates how to assess the safety and efficacy of a hyaluronidase treatment disclosed herein using an animal model for a filler-induced vascular occlusion event. New Zealand white rabbits will be used to simulate hyaluronic acid associated vascular occlusion(s) of an area of tissue. These animals will be selected due to the similarity of their vascular anatomy to that of humans. New Zealand white rabbits weighing 2.0 to 3.0 kg will be divided into groups based on the hyaluronidase dose and administration time.

Hyaluronic acid filler will be administered into an artery of animals to create an arterial occlusion. This arterial occlusion as well as subsequent ischemia will be confirmed by angiography and electroencephalogram (EEG) analyses. Different doses of hyper-concentrated hyaluronidase will be administered at several post-obstruction time points to assess both dosing and timing effectiveness of the treatment. Control animals will be injected with hyaluronic acid filler in the same manner as experimental animals but will receive no hyaluronidase treatment. Angiography and EEG changes will be recorded at 30, 60, 90, and 120 minutes after administration of hyaluronidase. EEG will be performed after 60 and 120 minutes to confirm the tissue reperfusion and electrophysiologic function.

In one series of experiments, an arterial occlusion will be induced by injecting 0.4 mL to 0.7 mL of hyaluronic acid filler into the left angular artery of all rabbits, with the right angular artery serving as an untreated control. The animals will then be divided into two experimental groups each containing 2 or 3 animals with Group 1 animals each receiving 500 μL of a composition comprising a low-concentration hyaluronidase (150 IU/mL) subdermally administered around the occlusion 30 minutes after the hyaluronic acid filler injection; and Group 2 animals each receiving 500 μL of a composition comprising a hyper-concentrated hyaluronidase (15,000 IU/mL) subdermally administered around the occlusion 30 minutes after the hyaluronic acid filler injection. The aforementioned dosing strategy for each set of rabbits is repeated every 30 minutes until the blood supply in each rabbit is no longer compromised. The number of vials/units of hyaluronidase that are injected into each rabbit will be recorded during the course of the study.

In another series of experiments, an arterial occlusion will be induced by injecting 0.6 mL to 0.9 mL of hyaluronic acid filler into the left internal carotid artery of all rabbits, with the right internal carotid artery serving as an untreated control. The animals will then be divided into two experimental groups each containing 2 or 3 animals with Group 1 animals each receiving 1 mL of a composition comprising a low-concentration hyaluronidase (150 IU/mL) administered in a retrobulbar fashion around the occlusion 30 minutes after the hyaluronic acid filler injection; and Group 2 animals each receiving 1 mL of a composition comprising a hyper-concentrated hyaluronidase (15,000 IU/mL) administered in a retrobulbar fashion around the occlusion 30 minutes after the hyaluronic acid filler injection. The aforementioned dosing strategy for each set of rabbits is repeated every 5 minutes until the blood supply in each rabbit is no longer compromised and the ERG for each animal returns to normal. The number of vials/units of hyaluronidase that are injected into each rabbit will be recorded during the course of the study.

In another series of experiments, an arterial occlusion will be induced by injecting 0.2 mL to 0.4 mL of hyaluronic acid filler into the left facial artery of all rabbits, with the right facial artery serving as an untreated control. The animals will then be divided into two experimental groups each containing 2 or 3 animals with Group 1 animals each receiving 250 μL of a composition comprising a low-concentration hyaluronidase (150 IU/mL) subdermally administered around the occlusion 30 minutes after the hyaluronic acid filler injection; and Group 2 animals each receiving 250 μL of a composition comprising a hyper-concentrated hyaluronidase (15,000 IU/mL) subdermally administered around the occlusion 30 minutes after the hyaluronic acid filler injection. The aforementioned dosing strategy for each set of rabbits is repeated every 30 minutes until the blood supply in each rabbit is no longer compromised. The number of vials/units of hyaluronidase that are injected into each rabbit will be recorded during the course of the study.

In another series of experiments, an arterial occlusion will be induced by injecting 0.2 mL to 0.4 mL of hyaluronic acid filler into the left infraorbital artery of all rabbits, with the right infraorbital artery serving as an untreated control. The animals will then be divided into two experimental groups each containing 2 or 3 animals with Group 1 animals each receiving 200 μL of a composition comprising a low-concentration hyaluronidase (150 IU/mL) subdermally administered around the occlusion 30 minutes after the hyaluronic acid filler injection; and Group 2 animals each receiving 200 μL of a composition comprising a hyper-concentrated hyaluronidase (15,000 IU/mL) subdermally administered around the occlusion 30 minutes after the hyaluronic acid filler injection. The aforementioned dosing strategy for each set of rabbits is repeated every 30 minutes until the blood supply in each rabbit is no longer compromised. The number of vials/units of hyaluronidase that are injected into each rabbit will be recorded during the course of the study.

In another series of experiments, an arterial occlusion will be induced by injecting 0.2 mL to 0.5 mL of hyaluronic acid filler into the left supratrochlear artery of all rabbits, with the right supratrochlear artery serving as an untreated control. The animals will then be divided into two experimental groups each containing 2 or 3 animals with Group 1 animals each receiving 300 μL of a composition comprising a low-concentration hyaluronidase (150 IU/mL) subdermally administered around the occlusion 30 minutes after the hyaluronic acid filler injection; and Group 2 animals each receiving 300 μL of a composition comprising a hyper-concentrated hyaluronidase (15,000 IU/mL) subdermally administered around the occlusion 30 minutes after the hyaluronic acid filler injection. The aforementioned dosing strategy for each set of rabbits is repeated every 30 minutes until the blood supply in each rabbit is no longer compromised. The number of vials/units of hyaluronidase that are injected into each rabbit will be recorded during the course of the study.

In another series of experiments, an arterial occlusion will be induced by injecting 0.1 mL to 0.3 mL of hyaluronic acid filler into the left supraorbital artery of all rabbits, with the right supraorbital artery serving as an untreated control. The animals will then be divided into two experimental groups each containing 2 or 3 animals with Group 1 animals each receiving 150 μL of a composition comprising a low-concentration hyaluronidase (150 IU/mL) subdermally administered around the occlusion 30 minutes after the hyaluronic acid filler injection; and Group 2 animals each receiving 150 μL of a composition comprising a hyper-concentrated hyaluronidase (15,000 IU/mL) subdermally administered around the occlusion 30 minutes after the hyaluronic acid filler injection. The aforementioned dosing strategy for each set of rabbits is repeated every 30 minutes until the blood supply in each rabbit is no longer compromised. The number of vials/units of hyaluronidase that are injected into each rabbit will be recorded during the course of the study.

In another series of experiments, an arterial occlusion will be induced by injecting 0.05 mL to 0.2 mL of hyaluronic acid filler into the left mental artery of all rabbits, with the right mental artery serving as an untreated control. The animals will then be divided into two experimental groups each containing 2 or 3 animals with Group 1 animals each receiving 100 μL of a composition comprising a low-concentration hyaluronidase (150 IU/mL) subdermally administered around the occlusion 30 minutes after the hyaluronic acid filler injection; and Group 2 animals each receiving 100 μL of a composition comprising a hyper-concentrated hyaluronidase (15,000 IU/mL) subdermally administered around the occlusion 30 minutes after the hyaluronic acid filler injection. The aforementioned dosing strategy for each set of rabbits is repeated every 30 minutes until the blood supply in each rabbit is no longer compromised. The number of vials/units of hyaluronidase that are injected into each rabbit will be recorded during the course of the study.

In another series of experiments, an arterial occlusion will be induced by injecting 0.1 mL to 0.3 mL of hyaluronic acid filler into the left superficial temporal artery of all rabbits, with the right superficial temporal artery serving as an untreated control. The animals will then be divided into two experimental groups each containing 2 or 3 animals with Group 1 animals each receiving 175 μL of a composition comprising a low-concentration hyaluronidase (150 IU/mL) subdermally administered around the occlusion 30 minutes after the hyaluronic acid filler injection; and Group 2 animals each receiving 175 μL of a composition comprising a hyper-concentrated hyaluronidase (15,000 IU/mL) subdermally administered around the occlusion 30 minutes after the hyaluronic acid filler injection. The aforementioned dosing strategy for each set of rabbits is repeated every 30 minutes until the blood supply in each rabbit is no longer compromised. The number of vials/units of hyaluronidase that are injected into each rabbit will be recorded during the course of the study.

In another series of experiments, an arterial occlusion will be induced by injecting 0.4 mL to 0.6 mL of hyaluronic acid filler into the left superficial temporal artery of all rabbits, with the right superficial temporal artery serving as an untreated control. The animals will then be divided into two experimental groups each containing 2 or 3 animals with Group 1 animals each receiving 350 μL of a composition comprising a low-concentration hyaluronidase (150 IU/mL) subdermally administered around the occlusion 30 minutes after the hyaluronic acid filler injection; and Group 2 animals each receiving 350 μL of a composition comprising a hyper-concentrated hyaluronidase (15,000 IU/mL) subdermally administered around the occlusion 30 minutes after the hyaluronic acid filler injection. The aforementioned dosing strategy for each set of rabbits is repeated every 30 minutes until the blood supply in each rabbit is no longer compromised. The number of vials/units of hyaluronidase that are injected into each rabbit will be recorded during the course of the study.

In another series of experiments, an arterial occlusion will be induced by injecting 0.1 mL to 0.3 mL of hyaluronic acid filler into the left radial artery of all rabbits, with the right radial artery serving as an untreated control. The animals will then be divided into two experimental groups each containing 2 or 3 animals with Group 1 animals each receiving 250 μL of a composition comprising a low-concentration hyaluronidase (150 IU/mL) subdermally administered around the occlusion 30 minutes after the hyaluronic acid filler injection; and Group 2 animals each receiving 250 μL of a composition comprising a hyper-concentrated hyaluronidase (15,000 IU/mL) subdermally administered around the occlusion 30 minutes after the hyaluronic acid filler injection. The aforementioned dosing strategy for each set of rabbits is repeated every 30 minutes until the blood supply in each rabbit is no longer compromised. The number of vials/units of hyaluronidase that are injected into each rabbit will be recorded during the course of the study.

In another series of experiments, an arterial occlusion will be induced by injecting 0.1 mL to 0.3 mL of hyaluronic acid filler into the left ulnar artery of all rabbits, with the right ulnar artery serving as an untreated control. The animals will then be divided into two experimental groups each containing 2 or 3 animals with Group 1 animals each receiving 125 μL of a composition comprising a low-concentration hyaluronidase (150 IU/mL) subdermally administered around the occlusion 30 minutes after the hyaluronic acid filler injection; and Group 2 animals each receiving 125 μL of a composition comprising a hyper-concentrated hyaluronidase (15,000 IU/mL) subdermally administered around the occlusion 30 minutes after the hyaluronic acid filler injection. The aforementioned dosing strategy for each set of rabbits is repeated every 30 minutes until the blood supply in each rabbit is no longer compromised. The number of vials/units of hyaluronidase that are injected into each rabbit will be recorded during the course of the study.

In another series of experiments, an arterial occlusion will be induced by injecting 0.4 mL to 0.6 mL of hyaluronic acid filler into the left femoral artery of all rabbits, with the right femoral artery serving as an untreated control. The animals will then be divided into two experimental groups each containing 2 or 3 animals with Group 1 animals each receiving 500 μL of a composition comprising a low-concentration hyaluronidase (150 IU/mL) subdermally administered around the occlusion 30 minutes after the hyaluronic acid filler injection; and Group 2 animals each receiving 500 μL of a composition comprising a hyper-concentrated hyaluronidase (15,000 IU/mL) subdermally administered around the occlusion 30 minutes after the hyaluronic acid filler injection. The aforementioned dosing strategy for each set of rabbits is repeated every 30 minutes until the blood supply in each rabbit is no longer compromised. The number of vials/units of hyaluronidase that are injected into each rabbit will be recorded during the course of the study.

In another series of experiments, an arterial occlusion will be induced by injecting 0.2 mL to 0.5 mL of hyaluronic acid filler into the left brachial artery of all rabbits, with the right brachial artery serving as an untreated control. The animals will then be divided into two experimental groups each containing 2 or 3 animals with Group 1 animals each receiving 350 μL of a composition comprising a low-concentration hyaluronidase (150 IU/mL) subdermally administered around the occlusion 30 minutes after the hyaluronic acid filler injection; and Group 2 animals each receiving 350 μL of a composition comprising a hyper-concentrated hyaluronidase (15,000 IU/mL) subdermally administered around the occlusion 30 minutes after the hyaluronic acid filler injection. The aforementioned dosing strategy for each set of rabbits is repeated every 30 minutes until the blood supply in each rabbit is no longer compromised. The number of vials/units of hyaluronidase that are injected into each rabbit will be recorded during the course of the study.

### Example 5

### Rabbit Model of Hypersensitivity Reaction

This example illustrates how to assess the safety and efficacy of a hyaluronidase treatment disclosed herein using an animal model for a hypersensitivity reaction. New Zealand white rabbits will be used to simulate a hyaluronic acid associated hypersensitivity reaction in an area of tissue. These animals will be selected due to the similarity of their vascular anatomy to that of humans. New Zealand white rabbits weighing 2.0 to 3.0 kg will be divided into groups based on the hyaluronidase dose and administration time.

In one series of experiments, animals suffering from a hypersensitivity reaction will be divided into four experimental groups each containing 2 or 3 animals with Group 1 animals each receiving 500 μL of a composition comprising a low-concentration hyaluronidase (150 IU/mL), 500 μL of 5-fluorouracil (5-FU), and 250 μL of 1% lidocaine subdermally administered around the occlusion 30 minutes after the hyaluronic acid filler injection; Group 2 animals each receiving 500 μL of a composition comprising a hyper-concentrated hyaluronidase (15,000 IU/mL), 500 μL of 5-fluorouracil (5-FU), and 250 μL of 1% lidocaine subdermally administered around the tissue exhibiting a hypersensitivity reaction; Group 3 animals each receiving 500 μL of a composition comprising a low-concentration hyaluronidase (150 IU/mL) and 250 μL of 1% lidocaine subdermally administered around the occlusion 30 minutes after the hyaluronic acid filler injection; and Group 4 animals each receiving 500 μL of a composition comprising a hyper-concentrated hyaluronidase (15,000 IU/mL) and 250 μL of 1% lidocaine subdermally administered around the tissue exhibiting a hypersensitivity reaction. The aforementioned dosing strategy for each set of rabbits is repeated every week until the hypersensitivity reaction is no longer noticeable in each rabbit.

### Example 6

### Rabbit Model of Filler-Induced Vascular Occlusion

In this example, New Zealand white rabbits were used to simulate hyaluronic acid polymer associated vascular occlusive loss of vision. These animals were selected due to the similarity of their ocular vascular anatomy to that of humans.

New Zealand white rabbits weighing 2.0 to 3.0 kg were acclimated to the study environment for a minimum of 1 week prior to the beginning of the study. At the completion of the acclimation period, each animal was physically examined for determination of suitability for the study including examination of the skin and external ears, eyes, abdomen, neurological, behavior, and general body condition. Animals determined to be in good health were released to the study. Released animals were allocated to four group of 3 animals each with each animal uniquely identified by a cage card number.

On the day of the surgical procedure (Day 0), all animals were anesthetized and dosed with an antibiotic. Each animal then underwent a surgical procedure to expose the expose the right carotid artery in the animal's neck. Using a 25-gauge or smaller needle attached to a syringe, 0.5 mL to 0.7 mL of a hyaluronic acid dermal filler was injected in the cranial direction into the right carotid artery over a minimum of 60 seconds. Once administration of the dermal filler was completed, the surgical incision site in the animal's neck was sutured closed. Approximately 5 minutes post-dermal filler administration, an eyelid speculum was positioned on the right eye of animals who then underwent one of four treatment protocols. Group 1 animals were injection controls that received no further treatment. Group 2-3 animals were each administered a composition by suprachoroidal injection using a 30-gauge needle in the inferior nasal quadrant of the right eye with Group 2 animals receiving 0.1 mL of Phospho-buffered saline (PBS) (control composition), and Group 3 animals receiving 0.1 mL of a hyaluronidase (2,400 IU/mL). Animals were allowed to recover normally from the anesthetic procedure. The left eye in all animals was untreated and served as a control in these experiments.

To assess visual activity, both optical coherence tomography angiography (OCT-A) and electroretinography (ERG) were performed. OCT-A is a non-contact retinal imaging system that uses infrared light to image retinal vasculature and determine vascular flow changes over time. In this study, each animal underwent OCT-A analysis to evaluate 1) pre-induction Day 0 versus post-induction Day 0 vascular flow changes; 2) pre-induction Day 0 versus post-induction Day 3 vascular flow changes; and 3) post-induction Day 0 versus post-induction Day 3 vascular flow changes. Full-field ERG was performed on both eyes of each animal on pre-induction Day 0 to obtain a baseline reading and on post-induction Day 3. Animals were dark adapted for at least 1 hour prior to ERG analysis. ERGs were elicited by brief flashes at 0.33 Hz delivered with a mini-ganzfeld photostimulator (Roland Instruments, Wiesbaden, Germany) at maximal intensity. Twenty responses were amplified, filtered, and averaged (Retiport Electrophysiologic Diagnostic Systems, Roland Instruments, Wiesbaden, Germany) for each animal. Animals underwent standard ERG measurements as dictated by ISCEV standards, including scotopic (0.01 candela), scotopic (3 candela), and photopic (3 candela) measurements. a-wave and b-wave ERG analysis was conducted to determine a-wave and b-wave amplitude changes over time, comparing pre-induction Day 0 (baseline) versus post-induction Day 3 under scotopic and photopic conditions.

At pre-induction Day 0, the right eye of Group 1 animals exhibited normal blood flow to the retina based on OCT-A analysis (Table 3) as well as normal neuronal activity in the retina based on ERG readings (Table 4). However, post-induction Day 3, all animals showed significantly reduced blood flow to the retina and ERG readings that were flat or essentially flat in the right eye (Tables 3 and 4) indicating a significant loss of blood flow and neuronal activity in the retina. As expected, the left eye of these animals showed normal blood flow and neuronal activity in the retina (Tables 3 and 4). Similarly, the right eye of Group 2 animals exhibited normal blood flow to the retina based on OCT-A analysis (Table 3) as well as normal neuronal activity in the retina based on ERG readings (Table 4). However, post-induction Day 3, all animals showed significantly reduced blood flow to the retina and ERG readings that were flat or essentially flat in the right eye (Tables 3 and 4) indicating a significant loss of blood flow and neuronal activity in the retina. As expected, the left eye of these animals showed normal blood flow and neuronal activity in the retina (Tables 3 and 4). As both Group 1 and Group 2 animals were controls, these results demonstrate that hyaluronic acid polymers can occlude blood flow to the retina and cause loss of retinal function and serve as a good model for hyaluronic acid polymer associated vascular occlusive loss of vision.

TABLE 3
Assessment of Blood Flow in Retina by OCT-A Analysis

| | Pre-induction Day 0 | | Post-induction Day 3 | |
|---|---|---|---|---|
| Animal | Left Eye | Right Eye | Left Eye | Right Eye |
| Group 1-1 | +++ | +++ | +++ | − |
| Group 1-2 | +++ | +++ | +++ | − |
| Group 1-3 | +++ | +++ | +++ | − |
| Group 2-1 | +++ | +++ | +++ | − |
| Group 2-2 | +++ | +++ | +++ | − |
| Group 2-3 | +++ | +++ | +++ | − |
| Group 3-1 | +++ | +++ | +++ | +++ |
| Group 3-2 | +++ | +++ | +++ | + |
| Group 3-3 | +++ | +++ | +++ | ++ |
| Group 3-4 | +++ | +++ | +++ | + |

+++ Normal blood flow measured in retina.
++ Near-normal blood flow measured in retina.
+ Low but significant blood flow measured in retina.
− No appreciable blood flow measured retina.

TABLE 4
Assessment of Neuronal Activity in Retina by ERG Analysis

| | Pre-induction Day 0 | | Post-induction Day 3 | |
|---|---|---|---|---|
| Animal | Left Eye | Right Eye | Left Eye | Right Eye |
| Group 1-1 | +++ | +++ | +++ | − |
| Group 1-2 | +++ | +++ | +++ | − |
| Group 1-3 | +++ | +++ | +++ | − |
| Group 2-1 | +++ | +++ | +++ | − |
| Group 2-2 | +++ | +++ | +++ | − |
| Group 2-3 | +++ | +++ | +++ | − |
| Group 3-1 | +++ | +++ | +++ | +++ |
| Group 3-2 | +++ | +++ | +++ | + |
| Group 3-3 | +++ | +++ | +++ | ++ |
| Group 3-4 | +++ | +++ | +++ | + |

+++ Normal neuronal activity measured in retina.
++ Near-normal neuronal activity measured in retina.
+ Low but significant neuronal activity measured in retina.
− No appreciable neuronal activity measured retina.

Analysis of Group 3 animals demonstrated that administration of hyaluronidase reversed hyaluronic acid polymer associated vascular occlusion in the retina. At pre-induction Day 0, the right eye of Group 3 animals exhibited normal blood flow to the retina based on OCT-A analysis (Table 3) as well as normal neuronal activity in the retina based on ERG readings (Table 4). These baseline measurements show that the animals in Group 3 exhibited functional neuronal activity. Surprisingly, post-induction Day 3, all animals also showed significant blood flow and neuronal activity in the retina (Tables 3 and 4). Although these levels were distinguishable from measurements obtained from the right eye on pre-induction Day 0 as well as measurements from the left eye control, the activity measured nonetheless illustrated a significant recovery from the vascular occlusion caused by the presence of hyaluronic acid polymers. These results demonstrate that hyaluronic acid polymers can occlude blood flow to the retina and cause loss of retinal function and serve as a good model for hyaluronic acid polymer associated vascular occlusive loss of vision. Furthermore, remarkably, small volumes of hyaluronidase administration into the suprachoroidal space cleared the hyaluronic acid dermal filler occlusion within the retinal circulation allowing for the reperfusion of the retinal tissue and reestablishment of neuronal activity. Lastly, these results indicate that the 2,400 IU/mL hyaluronidase treatment outperformed both 150 IU/mL hyaluronidase treatment and 1,500 IU/mL hyaluronidase treatment when injected into the suprachoroidal space, highlighting the ability of at least a 2,400 IU/mL hyaluronidase treatment to move from the SCS (outside the blood vessel) to inside the blood vessel(s) of the eye, which has important implications for treatment.

### Example 7

### Rabbit Model of Filler-Induced Vascular Occlusion

New Zealand red rabbits will be used to simulate hyaluronic acid associated vascular occlusive loss of vision. These animals will be selected due to the similarity of their ocular vascular anatomy to that of humans. New Zealand white rabbits weighing 2.0 to 3.0 kg will be divided into groups based on the hyaluronidase dose and administration time.

Hyaluronic acid filler will be administered into the internal carotid artery of animals to create a central retinal artery occlusion. This artery occlusion as well as subsequent ischemia will be confirmed by both retinal fundus photography, OCT-A, and ERG analyses. Different doses of hyaluronidase will be suprachoroidally administered at several post-obstruction time points to assess both dose and timing effectiveness of the treatment. Control animals will be injected with hyaluronic acid filler in the same manner as experimental animals but will receive no hyaluronidase treatment. Fundus photography and electroretinogram changes will be recorded at 30, 60, 90, and 120 minutes after administration of hyaluronidase. Electroretinography will be performed after 60 and 120 minutes to confirm the retinal reperfusion and electrophysiologic function.

In one series of experiments, a central retinal artery occlusion will be induced by injecting 0.5 mL to 0.9 mL of hyaluronic acid filler into rabbits, which will be divided into four experimental groups each containing 2 or 3 animals with Group 1 animals receiving suprachoroidally administered 100 μL hyaluronidase (400 IU/mL) 5 minutes after hyaluronic acid filler injection; Group 2 animals receiving suprachoroidally administered 100 μL hyaluronidase (400 IU/mL) 10 minutes after hyaluronic acid filler injection; Group 3 animals receiving suprachoroidally administered 100 μL hyaluronidase (600 IU/mL) 5 minutes after hyaluronic acid filler injection; and Group 4 animals receiving suprachoroidally administered 100 μL hyaluronidase (650 IU/mL) 10 minutes after hyaluronic acid filler injection.

In another series of experiments, a central retinal artery occlusion will be induced by injecting 0.5 mL to 0.9 mL of hyaluronic acid filler into rabbits, which will be divided into four experimental groups each containing 2 or 3 animals with Group 1 animals receiving suprachoroidally administered 100 μL hyaluronidase (1,000 IU/mL) 5 minutes after hyaluronic acid filler injection; Group 2 animals receiving suprachoroidally administered 100 μL hyaluronidase (1,000 IU/mL) 10 minutes after hyaluronic acid filler injection; Group 3 animals receiving suprachoroidally administered 100 μL hyaluronidase (3,000 IU/mL) 5 minutes after hyaluronic acid filler injection; and Group 4 animals receiving suprachoroidally administered 100 μL hyaluronidase (3,000 IU/mL) 10 minutes after hyaluronic acid filler injection.

In another series of experiments, a central retinal artery occlusion will be induced by injecting 0.5 mL to 0.9 mL of hyaluronic acid filler into rabbits, which will be divided into four experimental groups each containing 2 or 3 animals with Group 1 animals receiving suprachoroidally administered 100 μL hyaluronidase (10,000 IU/mL) 5 minutes after hyaluronic acid filler injection; Group 2 animals receiving suprachoroidally administered 100 μL hyaluronidase (10,000 IU/mL) 10 minutes after hyaluronic acid filler injection; Group 3 animals receiving suprachoroidally administered 100 μL hyaluronidase (15,000 IU/mL) 5 minutes after hyaluronic acid filler injection; and Group 4 animals receiving suprachoroidally administered 100 μL hyaluronidase (15,000 IU/mL) 10 minutes after hyaluronic acid filler injection.

In another series of experiments, a central retinal artery occlusion will be induced by injecting 0.5 mL to 0.9 mL of hyaluronic acid filler into rabbits, which will be divided into four experimental groups each containing 2 or 3 animals with Group 1 animals receiving suprachoroidally administered 100 UL hyaluronidase (15,000 IU/mL) 5 minutes after hyaluronic acid filler injection; Group 2 animals receiving suprachoroidally administered 100 μL hyaluronidase (15,000 IU/mL) 10 minutes after hyaluronic acid filler injection; Group 3 animals receiving suprachoroidally administered 100 μL hyaluronidase (20,000 IU/mL) 5 minutes after hyaluronic acid filler injection; and Group 4 animals receiving suprachoroidally administered 100 μL hyaluronidase (20,000 IU/mL) 10 minutes after hyaluronic acid filler injection.

In another series of experiments, a central retinal artery occlusion will be induced by injecting 0.5 mL to 0.9 mL of hyaluronic acid filler into rabbits, which will be divided into four experimental groups each containing 2 or 3 animals with Group 1 animals receiving suprachoroidally administered 100 μL hyaluronidase (400 IU/mL) 15 minutes after hyaluronic acid filler injection; Group 2 animals receiving suprachoroidally administered 100 μL hyaluronidase (400 IU/mL) 30 minutes after hyaluronic acid filler injection; Group 3 animals receiving suprachoroidally administered 100 μL hyaluronidase (650 IU/mL) 15 minutes after hyaluronic acid filler injection; and Group 4 animals receiving suprachoroidally administered 100 μL hyaluronidase (650 IU/mL) 30 minutes after hyaluronic acid filler injection.

In another series of experiments, a central retinal artery occlusion will be induced by injecting 0.5 mL to 0.9 mL of hyaluronic acid filler into rabbits, which will be divided into four experimental groups each containing 2 or 3 animals with Group 1 animals receiving suprachoroidally administered 100 μL hyaluronidase (1,000 IU/mL) 15 minutes after hyaluronic acid filler injection; Group 2 animals receiving suprachoroidally administered 100 μL hyaluronidase (1,000 IU/mL) 30 minutes after hyaluronic acid filler injection; Group 3 animals receiving suprachoroidally administered 100 μL hyaluronidase (3.000 IU/mL) 15 minutes after hyaluronic acid filler injection; and Group 4 animals receiving suprachoroidally administered 100 μL hyaluronidase (3,000 IU/mL) 30 minutes after hyaluronic acid filler injection.

In another series of experiments, a central retinal artery occlusion will be induced by injecting 0.5 mL to 0.9 mL of hyaluronic acid filler into rabbits, which will be divided into four experimental groups each containing 2 or 3 animals with Group 1 animals receiving suprachoroidally administered 100 μL hyaluronidase (10,000 IU/mL) 15 minutes after hyaluronic acid filler injection; Group 2 animals receiving suprachoroidally administered 100 μL hyaluronidase (10,000 IU/mL) 30 minutes after hyaluronic acid filler injection; Group 3 animals receiving suprachoroidally administered 100 μL hyaluronidase (15,000 IU/mL) 15 minutes after hyaluronic acid filler injection; and Group 4 animals receiving suprachoroidally administered 100 μL hyaluronidase (15,000 IU/mL) 30 minutes after hyaluronic acid filler injection.

In another series of experiments, a central retinal artery occlusion will be induced by injecting 0.5 mL to 0.9 mL of hyaluronic acid filler into rabbits, which will be divided into four experimental groups each containing 2 or 3 animals with Group 1 animals receiving suprachoroidally administered 100 μL hyaluronidase (15,000 IU/mL) 15 minutes after hyaluronic acid filler injection; Group 2 animals receiving suprachoroidally administered 100 μL hyaluronidase (15,000 IU/mL) 30 minutes after hyaluronic acid filler injection; Group 3 animals receiving suprachoroidally administered 100 μL hyaluronidase (20,000 IU/mL) 15 minutes after hyaluronic acid filler injection; and Group 4 animals receiving suprachoroidally administered 100 μL hyaluronidase (20,000 IU/mL) 30 minutes after hyaluronic acid filler injection.

Analysis of eyes from experimental animals by retinal fundus photography, OCT-A, and ERG analysis are expected to reveal no significant central retinal artery occlusion or loss of visual acuity after hyaluronidase treatment. Control animals, on the other hand, are expected to exhibit no improvement in filler-induced central retinal artery occlusion or visual acuity, suggesting loss of vision.

In closing, foregoing descriptions of embodiments of the present invention have been presented for the purposes of illustration and description. It is to be understood that, although aspects of the present invention are highlighted by referring to specific embodiments, one skilled in the art will readily appreciate that these described embodiments are only illustrative of the principles comprising the present invention. As such, the specific embodiments are not intended to be exhaustive or to limit the invention to the precise forms disclosed. Therefore, it should be understood that embodiments of the disclosed subject matter are in no way limited to a particular element, compound, composition, component, article, apparatus, methodology, use, protocol, step, and/or limitation described herein, unless expressly stated as such.

In addition, groupings of alternative embodiments, elements, steps and/or limitations of the present invention are not to be construed as limitations. Each such grouping may be referred to and claimed individually or in any combination with other groupings disclosed herein. It is anticipated that one or more alternative embodiments, elements, steps and/or limitations of a grouping may be included in, or deleted from, the grouping for reasons of convenience and/or patentability. When any such inclusion or deletion occurs, the specification is deemed to contain the grouping as modified, thus fulfilling the written description of all Markush groups used in the appended claims.

Furthermore, those of ordinary skill in the art will recognize that certain changes, modifications, permutations, alterations, additions, subtractions and sub-combinations thereof can be made in accordance with the teachings herein without departing from the spirit of the present invention. Furthermore, it is intended that the following appended claims and claims hereafter introduced are interpreted to include all such changes, modifications, permutations, alterations, additions, subtractions and sub-combinations as are within their true spirit and scope. Accordingly, the scope of the present invention is not to be limited to that precisely as shown and described by this specification.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

The words, language, and terminology used in this specification is for the purpose of describing particular embodiments, elements, steps and/or limitations only and is not intended to limit the scope of the present invention, which is defined solely by the claims. In addition, such words, language, and terminology are to be understood not only in the sense of their commonly defined meanings, but to include by special definition in this specification structure, material or acts beyond the scope of the commonly defined meanings. Thus, if an element, step or limitation can be understood in the context of this specification as including more than one meaning, then its use in a claim must be understood as being generic to all possible meanings supported by the specification and by the word itself.

The definitions and meanings of the elements, steps or limitations recited in a claim set forth below are, therefore, defined in this specification to include not only the combination of elements, steps or limitations which are literally set forth, but all equivalent structure, material or acts for performing substantially the same function in substantially the same way to obtain substantially the same result. In this sense it is therefore contemplated that an equivalent substitution of two or more elements, steps and/or limitations may be made for any one of the elements, steps or limitations in a claim set forth below or that a single element, step or limitation may be substituted for two or more elements, steps and/or limitations in such a claim. Although elements, steps or limitations may be described above as acting in certain combinations and even initially claimed as such, it is to be expressly understood that one or more elements, steps and/or limitations from a claimed combination can in some cases be excised from the combination and that the claimed combination may be directed to a sub-combination or variation of a sub-combination. As such, notwithstanding the fact that the elements, steps and/or limitations of a claim are set forth below in a certain combination, it must be expressly understood that the invention includes other combinations of fewer, more, or different elements, steps and/or limitations, which are disclosed in the specification even when not initially claimed in such combinations. Furthermore, insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. Accordingly, the claims are thus to be understood to include what is specifically illustrated and described above, what is conceptually equivalent, what can be obviously substituted, and also what essentially incorporates the essential idea of the invention.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as, e.g., “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising”, variations thereof such as, e.g., “comprise” and “comprises”, and equivalent open-ended transitional phrases thereof like “including”, “containing” and “having”, encompass all the expressly recited elements, limitations, steps, integers, and/or features alone or in combination with unrecited subject matter; the named elements, limitations, steps, integers, and/or features are essential, but other unnamed elements, limitations, steps, integers, and/or features may be added and still form a construct within the scope of the claim. Specific embodiments disclosed herein may be further limited in the claims using the closed-ended transitional phrases “consisting of” or “consisting essentially of” (or variations thereof such as, e.g., “consist of”, “consists of”, “consist essentially of”, and “consists essentially of”) in lieu of or as an amendment for “comprising.” When used in the claims, whether as filed or added per amendment, the closed-ended transitional phrase “consisting of” excludes any element, limitation, step, integer, or feature not expressly recited in the claims. The closed-ended transitional phrase “consisting essentially of” limits the scope of a claim to the expressly recited elements, limitations, steps, integers, and/or features and any other elements, limitations, steps, integers, and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps, integers, and/or features specifically recited in the claim, whereas the meaning of the closed-ended transitional phrase “consisting essentially of” is being defined as only including those elements, limitations, steps, integers, and/or features specifically recited in the claim and those elements, limitations, steps, integers, and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such, the embodiments described herein or so claimed with the phrase “comprising” expressly and unambiguously provide description, enablement, and support for the phrases “consisting essentially of” and “consisting of.”

Lastly, all patents, patent publications, and other references cited and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard is or should be construed as an admission that the inventors are not entitled to antedate such disclosure by virtue of prior invention or for any other reason. All statements as to the date or representation as to the contents of these documents are based on the information available to the applicant and do not constitute any admission as to the correctness of the dates or contents of these documents.

1. A method of reducing or eliminating a hyaluronic acid-induced blockage in one or more blood vessels supplying blood to an area of tissue in an individual in need thereof, the method comprising administering a composition comprising a therapeutically effective amount of a hyper-concentrated hyaluronidase of at least 15,000 IU/mL to the individual in a manner that reduces or eliminates a hyaluronic acid-induced blockage in one or more blood vessels supplying blood to an area of tissue being affected by the blockage.

2-3. (canceled)

4. The method according to claim 1, wherein the composition is administered to a dermal portion of a skin region.

5. (canceled)

6. The method according to claim 1, wherein the therapeutically effective amount of the hyper-concentrated hyaluronidase is in a concentration of at least 25,000 IU/mL.

7. The method according to claim 6, wherein the therapeutically effective amount of the hyper-concentrated hyaluronidase is in a concentration of at least 50,000 IU/mL.

8. The method according to claim 1, wherein the therapeutically effective amount of the hyper-concentrated hyaluronidase is in a concentration of about 25,000 IU/mL to about 500,000 IU/mL.

9-13. (canceled)

14. The method according to claim 1, wherein the composition is administered in a single dose or in multiple doses.

15. The method according to claim 1, wherein the composition is administered alone or in combination with a supplementary active compound, an agent, a drug, or a hormone.

16-44. (canceled)

45. A method of reducing or inhibiting a vascular occlusion in an area of tissue of an individual in need thereof, the method comprising administering a composition composition comprising a therapeutically effective amount of a hyper-concentrated hyaluronidase of at least 15,000 IU/mL to the individual in a manner that reduces or eliminates a hyaluronic acid-induced blockage in one or more blood vessels supplying blood to an area of tissue being affected by the blockage, thereby reducing or inhibiting the vascular occlusion in the area of tissue.

46. The method according to claim 45, wherein the composition is administered to a dermal portion of a skin region.

47. The method according to claim 45, wherein the therapeutically effective amount of the hyper-concentrated hyaluronidase is in a concentration of at least 25,000 IU/mL.

48. The method according to claim 47, wherein the therapeutically effective amount of the hyper-concentrated hyaluronidase is in a concentration of at least 50,000 IU/mL.

49. The method according to claim 45, wherein the therapeutically effective amount of the hyper-concentrated hyaluronidase is in a concentration of about 25,000 IU/mL to about 500,000 IU/mL.

50. The method according to claim 1, wherein the composition is administered alone or in combination with a supplementary active compound, an agent, a drug, or a hormone.

51. The method according to claim 45, wherein the composition is administered in a single dose or in multiple doses.

52. A method of reducing or inhibiting a hyaluronic acid-induced loss of tissue in an individual in need thereof, the method comprising administering a composition comprising a therapeutically effective amount of a hyper-concentrated hyaluronidase of at least 15,000 IU/mL to an individual in a manner that reduces or eliminates a hyaluronic acid-induced blockage in one or more blood vessels supplying blood to an area of tissue being affected by the blockage, thereby reducing or inhibiting the hyaluronic acid-induced loss of tissue in the individual.

53. The method according to claim 52, wherein the composition is administered to a dermal portion of a skin region.

54. The method according to claim 52, wherein the therapeutically effective amount of the hyper-concentrated hyaluronidase is in a concentration of at least 25,000 IU/mL.

55. The method according to claim 54, wherein the therapeutically effective amount of the hyper-concentrated hyaluronidase is in a concentration of at least 50,000 IU/mL.

56. The method according to claim 52, wherein the therapeutically effective amount of the hyper-concentrated hyaluronidase is in a concentration of about 25,000 IU/mL to about 500,000 IU/mL.

57. The method according to claim 52, wherein the composition is administered in a single dose or in multiple doses.

58. The method according to claim 52, wherein the composition is administered in a single dose or in multiple doses.
